(19) United States
(12) Patent Application Publication
(10) Pub. No.: US 2009/0239312 A1
(43) Pub. Date: Sep. 24, 2009

(54) Chromosome 6 and 9 Genes Involved in Premature Canities

(75) Inventors: Olivier De Lacharriere, Paris (FR); Jean-Louis Blouin, Ville-La-Grand (FR); Claire Deloche, Paris (FR); Stylianos Antonarakis, Geneve (CH)

Correspondence Address: BUCHANAN, INGERSOLL & ROONEY PC, POST OFFICE BOX 1404, ALEXANDRIA, VA, 22313-1404, US

(73) Assignee: L'OREAL, Paris (FR)

(21) Appl. No.: 12/474,244

(22) Filed: May 28, 2009

Related U.S. Application Data
(63) Continuation of application No. 11/031,012, filed on Jan. 10, 2005.
Application No. 11/031,012 is a continuation of application No. PCT/FR03/02154, filed on Jul. 9, 2003.

(30) Foreign Application Priority Data
Jul. 10, 2002 (FR) 0208696
Apr. 8, 2003 (FR) 0304360

(51) Int. Cl. G01N 33/566

(52) U.S. Cl. 436/501

(57) ABSTRACT

The invention provides a cosmetic or therapeutic method for combating canities and/or stimulating natural pigmentation and/or treating a pigmentation disorder comprising administering at least one polynucleotide fragment comprising 18 consecutive nucleotides, the sequence of which corresponds to all or part of a gene on human chromosome 9 selected from the group consisting of the FREQ, NT_030046.18, NT_030046.17, GTF3C5, CEL, CELL, FS, ABO, BARBLI, DDX31, GTF3C4 and Q96MA6 genes, or the sequence of which corresponds to all or part of a gene on human chromosome 6 selected from the HLAG, NT_007592.445, NT_007592.446, NT_007592.506, NT_007592.507, NT_007592.508, HSPA1 B, G8, NEU1, NG22, BAT8, HLA-DMB, HLA-DMA, BRD2, HLA-DQA1, HLA-DQA2, NT_007592.588, GRM4, RNF23, FLJ22638, NT_007592.459 and NT_007592.457 genes, and diagnostic methods employing same.

More and more people are becoming preoccupied with holding back or reversing the effects of ageing. In this context, causing white hair, which is deemed to be unsightly, to disappear by using a coloring treatment shampoo is now widely practised. However, while that technique can effectively remove the effects of the phenomenon, it has no effect whatsoever on the causes. For this reason, that solution is temporary and has to be repeated frequently.

In that context, the inventors have elected to explore the appearance of white hair or canities from a completely fresh angle, namely genetics.

Exploring canities from its genetic aspect brings to light the deep mechanisms of depigmentation. This means that genes which are involved in canities can be identified. This identification opens the door to a wide variety of applications, both cosmetic and therapeutic or diagnostic, in the field of hair care.

Investigating the genomic regions responsible for canities by genetic linkage analysis is entirely novel; past studies have attempted to decode the biochemistry of canities.

The inventors have elected to subscribe to the long-held hypothesis that premature canities (PC) or appearance of white hairs early in life is hereditary. The familial nature of premature whitening of the hair in some people is clearly observable.

The second obstacle to carrying out reverse genetics methods concerns the exact definition of phenotype. It is vital to have a complete definition of the phenotype being studied. To guarantee the best chances of success for this type of gene identification, selection and composition of the sample used in the present invention were made using a rigorous protocol for attributing phenotype and selecting families. The “premature canities” phenotype was attributed only to individuals who had some white hair before the age of 25 and for whom half of the hairs of the head were gray at 30 years of age.

Further, it is highly probable that firstly, premature canities is of a multigenic rather than a monogenic origin and secondly, that environmental factors have an influence on phenotype. In fact, a set of causes which give rise to a predisposition to premature canities has to be defined rather than a single mutation which is responsible for the phenotype. In that context, reverse genetics is not usually the technique of choice used by geneticists. Thus, the use of this method by the inventors is novel.

The results of these studies have allowed the inventors to define chromosomal and/or genomic zones comprising the genes most probably involved in canities. In the present application, chromosomal regions or sub-regions identified by the inventors as comprising genes which are statistically involved in canities, will indiscriminately be termed “chromosomal regions of the invention” or “genomic regions of the invention” or “chromosomal zones of the invention” or “genomic zones of the invention”. The genes identified in the context of the present invention within said regions will be termed the “genes of the invention”.

In a first aspect, the present invention concerns the genes of the chromosomal regions which have been identified and in a second aspect, the invention concerns the use of derived products such as transcription or translation products, in the fields of cosmetics, therapeutics and diagnostics.

Regarding the fields of therapy and cosmetics, the present invention successively concerns the use of polynucleotides deriving from a gene included in a chromosomal region of the invention, the use of agents which are capable of modifying the function attaching to that gene, the use of gene expression products and the use of agents that can modify the function of said expression products. The joint or combined use of at least two of the preceding products may prove to be judicious, particularly in the therapeutic field.

The present invention also concerns a method for diagnosing premature canities based on allelic variations in genes comprised in the chromosomal regions of the invention. Regarding diagnosis, it may also be particularly pertinent to combine the information deriving from the different genes of the chromosomal zones of the invention.

### GLOSSARY

The terms used in the context of the present invention have the following meanings:

The term “polynucleotide fragment” means any molecule resulting from a linear concatenation of at least two nucleotides, said molecule possibly being monocatenary, bicatenary or tricatenary. It may thus be a double-stranded DNA molecule, a single-stranded DNA molecule, an RNA molecule, a single strand DNA-RNA duplex, a DNA-RNA triplex or any other combination. The polynucleotide fragment may be a natural isolate, a recombinant or a synthetic molecule. When the polynucleotide fragment comprises complementary strands, the complementarity is not necessarily perfect, but the affinity between the different strands is sufficient to allow a stable Watson-Crick type bond to be established between the two strands.

Although the base pairing is preferably of the Watson-Crick type, other types such as Hoogsteen or reverse Hoogsteen type pairings are also possible.

The sequence S of a molecule is considered to “correspond” to the sequence of a given DNA molecule if the concatenation of the bases of S can be deduced from that of the given DNA molecule using one of the following methods:

1—by identity; or

2—by identity, but changing some or all of the thymine to uracil; or

3—by complementarity; or

4—by complementarity, but changing some or all of the thymine to uracil.

Furthermore, two sequences are considered to remain “corresponding” if globally they introduce less than one error in 10 in one of the preceding methods (complementarity or identity, with or without T/U exchange), preferably less than one error in 100. As a result, the two molecules also necessarily have similar lengths, the maximum variation in length being 10% according to the accepted error margin; preferably, the difference in length is less than 1%.

This definition does not assume that the two molecules are of the same nature, in particular as regards their backbone; it only concerns a correspondence in their sequences.

As an example, two identical DNA sequences “correspond” with each other. Similarly, if those two sequences are substantially identical, i.e. more than 90% identical, they correspond. An RNA sequence derived from translation of any DNA molecule “corresponds” to the sequence of that DNA molecule. Similarly, a synthetic sequence, for example a DNA-RNA hybrid, could correspond to a DNA sequence. The same is true between a DNA sequence and the anti-sense RNA sequence having that sequence as target.

On the same tack, the sequence S of a DNA molecule “corresponds” to the sequence of a given DNA molecule if the sequence S thereof can be deduced from that of the given DNA molecule using method 1 or 3 alone. The same latitude is permitted regarding the possibility of introducing errors into these processes, i.e. two DNA sequences remain “corresponding” if globally they introduce less than one error in 10 into the complementarity or identity processes, preferably less than one error in 100.

The term “expression products” of a DNA fragment encompasses all molecules that translate the genetic information carried by said fragment. RNA corresponding to transcription of the DNA fragment at all maturation stages is thus an expression product; this is the same for polypeptides at all stages of maturation resulting from the translation of RNA. If cleavages occur within the polypeptide, such as cleavage of addressing signals, all of the resulting polypeptides are also considered to be expression products of the initial DNA fragment.

Within the context of the invention, the primary “function” of a DNA fragment is preferably to be transcribed then translated into protein. The secondary function of the DNA can be assimilated to the function of the protein resulting from translation of said DNA. The function of a DNA fragment also has other meanings in the present invention. In particular, a DNA fragment may belong to a regulating region of a gene, and thus its function is to be the binding site for enhancers or inhibitors, or to be the binding site for RNA polymerase, or to be a recognition site for positioning RNA polymerase or any other function that can generally be assimilated with a regulating sequence.

Other functions can be envisaged for DNA fragments. In particular, their simple presence in a gene can facilitate recombination. Similarly, one function in accordance with the invention may be that of telomers and may be of significance in degeneracy. Other particular functions attributed to said DNA functions are well known to biologists.

A “genetic marker” is a detectable DNA sequence. In human genetics, markers are particular DNA sequences which can take different forms in different individuals. This marker polymorphism allows their transmission along genealogical branches to be followed.

Two major categories of conventional markers can be identified, namely microsatellite markers and SNPs (single nucleotide polymorphisms).

A microsatellite is a repeated DNA sequence constituted by a relatively simple motif, usually a di-, a tri- or a tetra-nucleotide. The number of repetitions changes for a given motif depending on the individual and can vary by several units (a minimum of a dozen for a di-nucleotide), up to over a hundred. Those sequences are dispersed throughout the genome, in an almost random manner, but at identical locations from one individual to another. They are highly abundant (about one every 10000 nucleotides=10 kb) and are highly polymorphic. The variation in the length of the tandem repeat constitutes the marker. Said microsatellite sequences are thus widely used as genetic markers.

Normally there is no explicit link between a microsatellite marker and a gene other than co-localization. According to current knowledge and apart from some rare cases of intragenic markers associated with certain diseases, the length of a tandem repeat is not linked to the role of the gene. In the context of the present invention, microsatellite makers are tools for localizing the genes involved in premature canities. As there is much less polymorphism in genes than in markers, a gene allele will be represented by several alleles of a single microsatellite marker.

Different methods exist for defining the localization of particular DNA sequences along chromosomes. The physical unit of measurement is the number of base pairs. However, the centimorgan is often used, and is a recombination unit and thus a genetic rather than a physical measurement. Two particular sequences of the same chromosome are separated by a centimorgan if they recombine once in a hundred times during meiosis. A centimorgan is approximately equivalent to 106 base pairs.

Another method for localizing particular DNA sequences along chromosomes consists of defining their position relative to markers which are evenly spaced along the chromosomes for which the position has been completely determined and is known. Markers which are widely used are microsatellite markers for which very complete maps exist. In particular, the GDB (genome database) is a database which is known worldwide for recording, inter alia, STSs (sequence tagged sites), which are specific unique limits of the DNA forming part of the microsatellites. A code DxSxxxx (for example D6S257) acts to identify said markers and is used as the accession number within the GDB. Said codes are a universal and unambiguous means of identification as only the GDB uses that type of code. As such microsatellite markers can be found about every 10 kb, it is thus possible to define the position of every sequence to about 10 kb, by indicating the microsatellite markers framing it.

An SNP (single nucleotide polymorphism) is a polymorphism which affects a single base in the DNA. This is the most widespread form of polymorphism in the human genome and is also characterized by high stability during transmission. The majority of said polymorphisms do not have functional implications. About 1 SNP is counted per 100 base pairs. Knowing those SNPs allows a map of the human genome to be established; SNPs thus serve as real genome markers; moreover, they are slow to mutate and have little chance of reappearing recurrently.

The term between two markers for a chromosomal region means the whole sequence between those two markers, limits included, and thus the sequence for the markers is included.

In reverse genetics, indices can localize a gene deriving from comparing transmission of a phenotype, which is assumed to be induced by a mutated gene or a given allele, with transmission of known markers, in the same family. Data regarding co-segregation of a phenotype and a marker allow genetic linkage analysis to be carried out.

Co-transmission of a phenotype and a marker suggests that the gene responsible for the phenotype and the marker are physically close to each other on the chromosome. The linkage is determined by analyzing the transmission model for a gene and a marker in the families carrying them.

Linkage analysis relies on the co-transmission of certain forms of markers with the defective or modified form of the gene. However, it is an indirect analysis in the sense that firstly, during a first step, a phenotype is associated with the defective or modified form of the gene. An error in assigning certain phenotypes vitiates the study. Secondly, that study is based on statistics, and those statistics rely on an analysis of a sample of the population and is thus a sampling method. Finally, it should be noted that when it is possible to associate a particular allele of the marker with an allele of a gene (in fact a phenotype), that association is only valid, a priori, for inter-familial samples.

The results of linkage analyses clearly depend on the degree of linkage between the marker and the locus of the disorder. Five centimorgans (5 cM) is considered to be a minimum linkage for a diagnosis. A 5 cM linkage means that there is a 95% chance of arriving at the correct conclusion and only a 1 in 20 chance of recombination occurring between the marker and the locus of the disorder.

The term gene as used in the present invention means not only the strictly encoding portion but also non-coding portions, such as the associated introns and the regulatory portions at the 5′ and 3′ ends, UTRs (untranslated regions), in particular the promoter or promoters, “enhancers” etc.

The inventors have identified two distinct chromosomal regions belonging to chromosomes 6 and 9 which are involved in premature canities. These two regions are each chromosomal regions or zones of the invention. More particularly, the inventors have determined the implication of certain genes belonging to these chromosomal regions, termed the genes of the invention.

In a first aspect, the invention concerns the genes HLAG, NT_{—}007592.445, NT_{—}007592.446, NT_{—}007592.506, NT_{—}007592.507, NT_{—}007592.508, HSPA1B, G8, NEU1 (neuraminidase precursor), NG22, BAT8 (ankyrin repeat-containing protein), HLA-DMB, HLA-DMA, BRD2, HLA-DQA1, HLA-DQA2, NT_{—}007592.588, GRM4 (glutamate receptor, metabotropic 4), RNF23, FLJ22638, NT_{—}007592.459 and NT_{—}007592.457 on human chromosome 6 identified by the inventors as being involved in premature canities and the uses of products derived from said genes, such as transcription or expression products. These genes form part of the first chromosomal zone of the invention which is delimited on chromosome 6 by the microsatellite markers D6S1629 and D6S257 More particularly, this zone is termed the “first chromosomal zone of the invention”.

Preferred genes from the genes cited above within the first chromosomal zone of the invention are the genes HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588. Particularly preferred genes are the NT_{—}007592.506, NT_{—}007592.507 and NT_{—}007592.508 genes.

In a second aspect, the invention concerns the genes FREQ (frequenin homolog), NT_{—}030046.18, NT_{—}030046.17, GTF3C5 (general transcription factor IIIC, polypeptide 5), CEL (carboxyl ester lipase bile salt-stimulated), CELL (carboxyl ester lipase-like bile salt-stimulated), FS (Forssman synthetase), ABO blood-group (transferase A, alpha), BARHL1, DDX31, GTF3C4 and Q96MA6 on human chromosome 9 identified by the inventors as being involved in premature canities, and uses of products derived from said genes, such as transcription or expression products. These genes belong to the second chromosomal zone of the invention which is delimited on chromosome 9 by the microsatellite marker D9S290 and the telomeric region (long arm telomere). More particularly, this zone will be termed the “second chromosomal zone of the invention”.

Preferred genes from the genes cited above in the second chromosomal zone of the invention are the genes BARHL1, DDX31, GTF3C4 and Q96MA6. More particularly preferred genes are the genes DDX31 and GTF3C4.

For the two chromosomal zones identified above, the present invention encompasses polynucleotide fragments with a minimum length of 18 nucleotides the sequence of which at least partially corresponds to one of the genes on human chromosome 9 selected from the FREQ, NT_{—}030046.18, NT_{—}030046.17, GTF3C5, CEL, CELL, FS, ABO, BARHL1, DDX31, GTF3C4 and Q96MA6 genes, or to one of the genes on human chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes. These polynucleotide fragments are also characterized by their involvement in canities or in premature canities, and possibly in both phenomena. The genes involved in premature canities are very probably also involved in age-related canities.

In accordance with one possibility envisaged by the present invention, a fragment involved in canities or premature canities and having a sequence satisfying the requirements mentioned above can be used in therapy.

More particularly, in a first aspect, the invention concerns genes on human chromosome 6 identified by the inventors as being involved in premature canities. In accordance with this aspect, a fragment encompassed by the invention has a sequence corresponding to all or part of a gene on human chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes. These genes are included in the first chromosomal zone of the invention delimited on the chromosome 6 by the microsatellite markers D6S1629 and D6S257.

Preferably, a fragment of the invention has a sequence corresponding to all or part of a gene selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588 genes. More preferably, the gene is selected from the NT_{—}007592.506, NT_{—}007592.507 and NT_{—}007592.508 genes.

The second aspect of the invention concerns genes on human chromosome 9 identified by the inventors as being involved in premature canities. In accordance with this aspect of the invention, a fragment encompassed by the invention has a sequence corresponding to all or part of a gene on human chromosome 9 selected from the FREQ, NT_{—}030046.18, NT_{—}030046.17, GTF3C5, CEL, CELL, FS, ABO, BARHL1, DDX31, GTF3C4 and Q96MA6 genes. Said genes are included in the second chromosomal zone of the invention delimited on chromosome 9 by the microsatellite marker D9S290 and the telomeric region (long arm telomere).

Preferably, a fragment of the invention has a sequence corresponding to all or part of the gene selected from the BARHL1, DDX31, GTF3C4 and Q96MA6 genes. More preferably, the gene is selected from the genes DDX31 and GTF3C4.

The polynucleotide fragment referred to in the invention corresponds to a fragment of a chromosome. This fragment has a minimum length of 18 nucleotides, and a maximum length which can be up to the total length of the gene in question, or several genes of the invention which are contiguous within the chromosomal region. Preferably, the fragment has more than 18 nucleotides. A particularly preferred length is in the range 18 to 10000 nucleotides, more preferably in the range 30 to 8000 nucleotides.

In accordance with preferred variations of the invention, reference is made to fragments the length of which is in the range 30 to 5000 nucleotides, preferably in the range 50 to 3000 nucleotides, for example in the range 100 to 2000 nucleotides, or in the range 200 to 1000 nucleotides.

The invention also concerns the use in cosmetics or therapy of a polynucleotide fragment or the expression product of a fragment or an agent modulating the function of a fragment, or of an agent modulating the function of the expression product of a fragment, where the fragment in question corresponds to all or part of a gene of human chromosome 9 selected from the FREQ, NT_{—}030046.18, NT_{—}030046.17, GTF3C5, CEL, CELL, FS, ABO, BARHL1, DDX31, GTF3C4 and Q96MA6 genes or to all or part of a gene on human chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes of one of the two chromosomal zones of the invention. In a preferred case, the fragment more particularly corresponds to a portion of an exon of one of said genes.

In the following text, the term “products of the invention” will be used to designate the fragment, the expression product of a fragment, the agent modulating the function of a fragment, and the agent modulating the function of the expression product of a polynucleotide fragment corresponding to all or part of one of the 22 genes of chromosome 6 or one of the 12 genes on chromosome 9 identified by the inventors.

For the genes identified by the inventors, the present invention firstly concerns uses in the cosmetics field. The term “cosmetics” means any application which modifies only esthetics and is not therapeutic in nature.

Regarding all of the uses of the invention in the cosmetics field, the product of the invention can be packaged in various appropriate forms, alone or in combination with other agents. In particular, preferred forms are intended for local application and are in the form of creams, lotions, gels, emulsions, pomades and shampoos. Other forms can also be envisaged for the uses of the invention, in particular in the form of pills for oral administration.

Of the different cosmetic aims in the context of the invention, a particularly preferred area is that of pigmentation. The pigmentation may be that of the skin or of the phanera, and may concern the color of the pigmentation or the absence of pigmentation; problems affecting the quality and intensity of pigmentation are also affected by the present invention.

In particular, the invention is aimed at using at least one product of the invention to prevent and/or limit and/or arrest the development of canities.

The invention also encompasses the use of at least one product of the invention to encourage natural pigmentation of gray hair of the head and/or body.

The present invention also pertains to a cosmetic method for treating canities, characterized in that a composition comprising at least one product of the invention is applied to the zone to be treated.

The invention also pertains to a cosmetic treatment method to encourage the natural pigmentation of gray or white hair of the head and/or body, characterized in that a composition comprising at least one product of the invention is applied to the zone to be treated

Non-limiting examples of the zones to be treated are the scalp, eyebrows, mustache and/or beard.

More particularly, the methods for treating canities and the natural pigmentation of gray or white head and/or body hair consist of application of a composition comprising at least one product of the invention.

Treatment methods for combating canities and/or stimulating the natural pigmentation of gray or white head and/or body hair can, for example, consist of applying the composition to the hair and scalp at night, leaving the composition in contact overnight and then optionally shampooing in the morning or washing the hair with said composition and leaving it in contact for a few minutes before rinsing. The composition of the invention has been shown to be particularly advantageous when applied in the form of a hair lotion, which may be rinsed out, or even in the form of a shampoo.

Regarding the genes identified by the inventors, the present invention then concerns therapeutic uses in the field of pigmentation.

Disorders affecting the pigmentation system, whether of the skin or phanera, can have severe consequences on the health of affected persons. Skin pigmentation acts as a barrier to attack by light; in particular, persons suffering from albinism are deprived of protection against sunlight, which constitutes a major danger to them. Other disorders involving pigmentation are also encompassed by the present invention.

In the context of therapeutic and cosmetic uses that can modify a characteristic of pigmentation, we preferably refer to skin pigmentation. In other cases envisaged by the present invention, the type of pigmentation which is to be modified concerns the pigmentation of phanera, in particular the nails or body hair.

In a particularly preferred case of the present invention, the pigmentation the characteristics of which are to be modified is that of the hair system in general and the hair of the head, mustache and eyebrows in particular. The present invention can modify the phenomenon whereby pigmentation of the hair of the head is halted, namely canities, in particular when it occurs prematurely in a person, whereupon we speak of premature canities.

For all therapeutic uses, the active products in the composition of a medicament are preferably associated with pharmaceutically acceptable excipients. Any administrative route which is considered acceptable can be used in the context of the invention, in particular intradermal, intravenous, muscular, oral, otic, nasal or optical. The formulation is preferably adapted to the selected administrative route.

Uses for manufacturing a medicament of the invention may involve other active principles in their formulations. Similarly, administration of a medicament as defined in the invention can be combined with administering another medicament, whether said administration is simultaneous, sequential or separate.

The various products used in the context of therapeutic uses can be combined and form part of the composition of a single medicament, or they may be employed in the manufacture of various medicaments. In particular, if they form part of the composition of distinct medicaments, they may be administered at different frequencies.

The preferred features and variations of the products employed in the uses of the invention may be identical in the context of their uses in cosmetology and for the uses of the same product in the manufacture of a medicament.

In both cases, the use of the products of the invention may require the product to be introduced into a body fluid or into the body tissues or into the cells. For introduction into cells, it may be necessary for the product to be active in the cell cytoplasm or in the cell nucleus.

The first use in cosmetics or therapeutics envisaged in the context of the invention is the use of a polynucleotide fragment the sequence of which at least partially corresponds to one of the genes on human chromosome 9 selected from the FREQ, NT_{—}030046.18, NT_{—}030046.17, GTF3C5, CEL, CELL, FS, ABO, BARHL1, DDX31, GTF3C4 and Q96MA6 genes or to one of the genes of the human chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes. For therapeutic uses, the polynucleotide fragment is used in the manufacture of a medicament.

In the context of said first use in accordance with the invention, preferred genes on chromosome 9 are BARHL1, DDX31, GTF3C4 and Q96MA6, more particularly the genes DDX31 and GTF3C4. Preferred genes on chromosome 6 are HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588, and more particularly NT_{—}007592.506, NT_{—}007592.507 and NT_{—}007592.508.

Regarding the chemical nature of this polynucleotide fragment, it may be a single or double stranded, circular or linear DNA molecule, an RNA molecule or any other molecule envisaged in the definition of the polynucleotide fragment given above.

Regarding its environment, said fragment may be or may form part of a plasmid, a viral genome or another type of vector. In other cases, it may form part of the genome of a cell, or of a cell which has been genetically modified to include that fragment in its genome. It may also be an isolated molecule.

Regarding regions surrounding said fragment, it is preferably under the control of regulating sequences. If the fragment is inserted in a vector, said vector preferably includes all of the sequences necessary for transcription and possibly translation of the fragment. Said fragment can also be surrounded by flanking regions which allow a step for homologous recombination with a further polynucleotide fragment, possibly resulting in insertion of the fragment of the invention into the genomic DNA of a target cell.

The polynucleotide fragment as described may be in its natural form or it may be synthetic in nature, or it may be partly one and partly the other, in particular if it is a “duplex” molecule constituted by two strands of different origins. In the different cases envisaged by the present invention, the polynucleotide fragment can be isolated; it may have undergone a purification step. It may also be a recombinant fragment, for example one synthesized in another organism. In a preferred example, it is a DNA fragment which has been amplified by PCR (polymerase chain reaction) then purified.

In other constructions envisaged by the present invention, the first use employs a polynucleotide fragment associated with a probe. This characteristic can, inter alia, allow the localization of the fragment to be followed from the extracellular medium to the cell, or from the cytoplasm to the nucleus, or it can allow its interaction with DNA or RNA or proteins to be determined. The probe can also enable degradation of the fragment to be monitored. The probe is preferably fluorescent, radioactive or enzymatic in nature. The skilled person will know which type of probe is best adapted to the characteristic which is to be monitored.

The polynucleotide fragment employed in the context of this first use of the invention can be used in a hybridization test, in a sequencing test, in a microsequencing test or in a mis-pairing detection test.

Said fragment of the invention contains at least 18 successive nucleotides, said 18 nucleotides constituting a sequence which corresponds to all or part of one of the 22 genes of the invention on human chromosome 6 or to all or part of one of the 11 genes of the invention on human chromosome 9, preferably all or part of the DDX31 or GTF3C4 gene. In particular, a fragment of the invention may contain only 18 complementary bases of 18 successive bases of one of the genes described above.

In a further particular case, the fragment described may be cDNA or RNA of one of the genes described above. It may correspond to one or more exons of one of the genes, and may correspond to a regulating sequence for one of the genes identified on chromosomes 6 and 9.

In the context of said first use, the number of polynucleotide fragments as defined above is not limited and is not necessarily restricted to a single fragment.

In particular, it may employ a plurality of polynucleotide fragments the sequence of which at least partially corresponds to all or part of a gene on human chromosome 6 selected from the HLAG, NT_{—}007592.445, NT 007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes. Preferably, the sequences of the different fragments correspond to distinct genes or to distinct exons.

Alternatively, it may employ a plurality of polynucleotide fragments the sequence of which at least partially corresponds to all or part of a gene on human chromosome 9 selected from the FREQ, NT_{—}030046.18, NT_{—}030046.17, GTF3C5, CEL, CELL, FS, ABO, BARHL1, DDX31, GTF3C4 and Q96MA6 genes. Preferably, the sequences of the different fragments correspond to distinct genes or to distinct exons.

This first use of the invention is preferably in the cosmetics field.

Said use can also enable the manufacture of a medicament for a therapeutic action in the pigmentation field.

In a particular case, the first use described involves a genetic modification whether or not it is induced by a nucleotide fragment as described.

In the case in which a gene responsible for pigmentation is defective as it has mutated, said first use of the invention can restore the function of that gene by introducing a polynucleotide fragment which represents a new wild-type copy of the defective endogenic gene.

When gene activation is responsible for depigmentation, said first use of the invention can abolish the function of the gene by introducing an antisense RNA which will block translation of the gene.

In a second use envisaged by the present invention, in the field of therapy and cosmetics, an agent modulating the function of a DNA fragment corresponding at least in part to one of the genes identified in the context of the present invention is used. For therapeutic uses, this defined agent is involved in the manufacture of a medicament. Preferably, the DNA fragment has at least 18 nucleotides.

Said agent of the invention may be capable of modulating the function of an exogenic DNA fragment a portion of the sequence of which corresponds to one of the genes identified by the inventors, or it may be capable of modulating the function of an endogenic sequence included in one of the genes identified by the invention. Preferably, an agent acting in this second use in accordance with the invention not only modulates the function of an exogenic DNA fragment as defined, but also of the corresponding endogenic DNA fragment.

The DNA fragment the function of which is modulated may partially correspond to one of the genes on chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes. Alternatively, the DNA fragment the function of which is modulated may correspond partially to one of the genes on chromosome 9 selected from the FREQ, NT_{—}030046.18, NT_{—}030046.17, GTF3C5, CEL, CELL, FS, ABO, BARHL1, DDX31, GTF3C4 and Q96MA6 genes. In particular, it may be a plasmid having just one short sequence corresponding to one of the chromosomal regions mentioned. Preferably, the sequence correspondence is established over at least 18 successive nucleotides.

In the context of said second use of the invention, the preferred genes on chromosome 9 are BARHL1, DDX31, GTF3C4 and Q96MA6, more particularly the DDX31 and GTF3C4 genes. Preferred genes on chromosome 6 are HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588, more particularly the NT_{—}007592.506, NT_{—}007592.507 and NT_{—}007592.508 genes.

The above statements in the definition section regarding the meaning of the “function of a DNA fragment” are applicable regarding envisaging all of the uses corresponding to a second use of the invention.

Given the plurality of functions of DNA fragments, modulation of said fragments encompasses very different aspects. In the particular case in which the function of said fragment is to be transcribed, modulating said function consists of encouraging or inhibiting the capacity of said fragment to be transcribed. It may also consist of modifying the transcription initiation and termination sites, or modifying the degree of transcription initiation. In another case, modulating the function may also consist of modifying RNA splicing, for example by modifying DNA recognition sequences responsible for distribution between introns and exons.

When the DNA fragment is part of a regulating sequence, modifying its function may consist of inhibiting binding of enhancers or inhibitors. In contrast, it may consist of encouraging binding, or encouraging binding of other transcription factors. This is also the case with sequences used by RNA polymerase.

In the context of said use in accordance with the invention, as is the case with all other uses in accordance with the invention, the number of products, in this case agents modulating the function of a DNA fragment corresponding at least in part to one of the genes identified in the present invention, is not limited and may be greater than one.

However, in the context of this second use, the different agents used have in common that they all modulate the function of DNA fragments the sequence of which belongs to or corresponds at least in part to the same gene of the invention. In accordance with a first aspect of the invention, said gene is one of 22 genes of the invention on human chromosome 6. In a second aspect of the present invention, the gene in question is one of 12 genes of the invention on human chromosome 9, preferably DDX31 or GTF3C4.

Examples of agents of the invention are single strand DNA molecules which can bind to defined sub-regions in one of the genes of the invention, to form triple helices. Under said conditions, agents of the invention destroy the function of the sub-region to which they hybridize.

Other preferred agents of the invention are polypeptides capable of interacting with defined sub-regions of one of the genes of the invention. Preferably, the agents of the invention are enhancers or inhibitors which bind to regulating regions of one of 22 genes of the invention on human chromosome 6.

Alternatively, agents of the invention are enhancers or inhibitors binding to regulating regions of one of the 12 genes of the invention on human chromosome 9, preferably DDX31 or GTF3C4.

A further category of the agents of the invention concerns molecules capable of interacting with precise regions along the DNA to change its conformation. A further category concerns molecules interacting with inhibitors or enhancers to modify their function, the inhibitors or enhancers having the initial function of modifying the expression of DNA fragments belonging to one of the genes identified in the context of the present invention.

An agent used in accordance with said second use of the invention can in particular modulate the function of a DNA fragment corresponding to 18 successive bases of one of the genes described above.

This second use of the invention is preferably in the cosmetics field. Said use can also allow a medicament to be manufactured for a therapeutic action in the pigmentation field.

In a particular case, this second use involves genetic modification whether or not induced by an agent modulating the function of a DNA fragment as described.

When gene activation is responsible for depigmentation, said second use of the invention can abolish the function of said gene by introducing an agent which will block translation of said gene by binding to its promoter region, for example.

When inactivation of a gene is responsible for depigmentation, said second use of the invention can restore the function of said gene by introducing an agent which will activate gene transcription, for example by binding to its promoter region, or by binding to an inhibitor which will thus stop inactivating that gene.

A third use envisaged by the present invention in the field of therapeutics and cosmetics is the use of an agent modulating the function of an expression product of a DNA fragment corresponding at least in part to one of the genes of the invention. For therapeutic uses, the agent defined is involved in the manufacture of a medicament. Preferably, the DNA fragment has at least 18 nucleotides.

In particular, such an agent of the invention modulates the function of a transcript from a DNA fragment which corresponds at least in part to one of the genes on chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes. In another case, an agent of the invention modulates the function of a polypeptide from translation of one of the transcripts mentioned. Alternatively, the DNA fragment the function of the expression product of which is modulated may correspond at least in part to one of the genes of the chromosome 9 selected from the FREQ, NT_{—}030046.18, NT_{—}030046.17, GTF3C5, CEL, CELL, FS, ABO, BARHL1, DDX31, GTF3C4 and Q96MA6 genes.

In the context of said third use of the invention, preferred genes on chromosome 9 are BARHL1, DDX31, GTF3C4 and Q96MA6, more particularly the DDX31 and GTF3C4 genes. Preferred genes on chromosome 6 are HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588, more particularly the NT_{—}007592.506, NT_{—}007592.507 and NT_{—}007592.508 genes.

Said agent of the invention may be capable of modulating the function of the expression product of an exogenic DNA fragment a portion of the sequence for which corresponds to one of the genes of the invention identified by the inventors, or it may be capable of modulating the function of the expression product of an endogenic sequence included in the genes of the invention. Preferably, an agent acting in this third use of the invention not only modulates the function of an expression product of an exogenic DNA fragment as defined above, but also of the corresponding endogenic DNA fragment.

The polypeptide's function can be modulated in different manners. In particular, its activity, yield, specificity, avidity for an antibody can be increased or decreased, its substrate can be modified for an enzyme, and its degree of conversion can be modified.

Preferred agents of the invention are RNA molecules, termed antisense RNA, which hybridize with at least one transcript from a DNA fragment corresponding at least in part to one of the genes identified by the inventors on chromosome 6, or to one of the genes identified by the inventor on chromosome 9. Other agents fulfilling the same roles may be single strand DNA molecules or hybrid DNA-RNA molecules. The role of said agents of the invention is preferably to encourage, prevent, retard, accelerate or introduce errors into translation of said transcript.

Other preferred agents of the invention belong to the polypeptide class. In particular, the invention concerns proteins that can bind to said transcript and thus modulate its translation. Such agents from the polypeptide class may be of natural or synthetic origin (synthesized chemically or biotechnologically). In particular, it may be an antibody. As mentioned above, said modulation can result in encouraging, preventing, retarding, accelerating or introducing errors into translation of said transcript. In particular, the interaction between the polypeptides and said transcript may constitute an obstacle to normal ribosome binding.

Agents as defined in the present invention may modulate the function of the protein encoded by a DNA fragment corresponding at least in part to one of the 22 genes of the invention on human chromosome 6. Said agents may or may not be proteic in nature. An agent of the invention may intervene at an early stage, preventing correct folding of the protein. An agent of the invention may also modify the function of said protein by modifying the three-dimensional structure after folding. It is also possible for said agent to be a protein inhibitor, in particular a competitive inhibitor.

In accordance with a further aspect of the invention, agents as defined in the present application may modulate the function of the protein encoded by a DNA fragment corresponding at least in part to one of the 12 genes of the invention from human chromosome 9, preferably DDX31 or GTF3C4.

Agents which may be suitable in the context of the present invention are not limited to those cited above.

In the context of said third use, the number of agents modifying the function of an expression product as defined above is not limited and is not necessarily restricted to a single agent.

However, in the context of said third use, the different agents employed therein may have in common the fact that they all modulate the function of expression products of DNA fragments the sequence for which belongs to or corresponds at least in part to the same gene of the invention In accordance with the first aspect of the invention, said gene is selected from the 22 genes of the invention on human chromosome 6. In accordance with the second aspect of the present invention, the gene in question is selected from the 12 genes of the invention on chromosome 9, preferably DDX31 or GTF3C4.

This third use of the invention is preferably in the cosmetics field. This use can also allow the manufacture of a medicament for therapeutic use, in the pigmentation field.

In a particular case, the third described use involves a genetic modification whether or not introduced by an agent modulating the function of an expression product of a DNA fragment as described herein.

In the case in which activation of the gene is responsible for depigmentation, said third use of the invention can destroy the function of that gene by introducing an antisense DNA which will block translation of said gene by preventing RNA-protein passage. A further preferred situation consists of selecting as the agent an antibody that is capable of binding to the protein resulting from translation of said gene.

A fourth use envisaged by the present invention in the therapeutic field and in the cosmetics field is the use of an expression product of a DNA fragment corresponding at least in part to one of the genes identified in the context of the present invention. For therapeutic uses, the agent defined above is used in the manufacture of a medicament. Preferably, the DNA fragment has at least 18 nucleotides.

In particular, said expression product is the RNA transcript whatever the maturation stage of said transcript, derived from a DNA fragment corresponding at least in part to one of the genes on chromosome 6 selected from HLAG, NT_{—}007592.445, NT_{—}007592.446, NT_{—}007592.506, NT_{—}007592.507 NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes, or one of the genes on chromosome 9 selected from the FREQ, NT 030046.18, NT_{—}030046.17, GTF3C5, CEL, CELL, FS, ABO, BARHL1, DDX31, GTF3C4 and Q96MA6 genes. In the case of splicing, the transcript can thus be smaller than the DNA fragment from which it is derived. Preferably, if the expression product is a RNA molecule, it comprises at least 18 nucleotides.

In the context of said fourth use of the invention, preferred genes on chromosome 9 are BARHL1, DDX31, GTF3C4 and Q96MA6, more particularly the DDX31 and GTF3C4 genes. Preferred genes on chromosome 6 are HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588, and more particularly the NT_{—}007592.506, NT_{—}007592.507 and NT_{—}007592.508 genes.

In a further preferred case, an expression product of the invention is derived from translation of one of the transcripts mentioned above. Said expression product can thus comprise less than 6 amino acids if the transcript from which it is derived has undergone splicing steps. Preferably, a peptide expression product contains at least 6 amino acids.

The expression product of the invention does not necessarily derive from the steps of transcription or translation of genomic DNA. In particular, an expression product used in accordance with the invention can be an expression product from exogenic DNA at least a portion of the sequence of which corresponds to part of one of the genes of the invention.

The present invention also envisages the use of a completely synthetic agent which is similar to the expression product of an exogenic or endogenic DNA fragment corresponding at least in part to one of the genes of the invention.

Preferred expression products for use in the present invention are RNA molecules, termed antisense RNA, which hybridize with at least one transcript from a DNA fragment corresponding at least in part to one of the genes identified by the inventors on chromosome 6, or to one of the genes identified by the inventors on chromosome 9. In particular, to form antisense RNAs having a specific RNA as the target, it is possible to use RNA from transcription of the same sequence of DNA as the target but not of the leader strand, but of its complementary sequence carried by the other strand. This produces RNA fragments that are complementary to target fragments normally synthesized by the cell. The expected role of said expression products of the invention is preferably to encourage, prevent, retard, accelerate or introduce errors in the translation of transcripts normally synthesized by the cell.

Other preferred expression products belong to the polypeptide class. In particular, the invention concerns proteins that are capable of introducing a change into the function of the cell in which they are active.

In the context of this use of the invention, the number of expression products of a DNA fragment the sequence of which belongs to or corresponds at least in part to one of the genes of the invention is not limited and may be greater than one.

However, in the context of this fourth use, the different products employed have in common the fact that they are all expression products of DNA fragments the sequence of which belongs to or corresponds at least in part to the same gene of the invention. In the first aspect of the invention, said gene is selected from the 22 genes of the invention on human chromosome 6. In accordance with the second aspect of the invention, the gene in question is selected from the 12 genes of the invention on chromosome 9, preferably DDX31 and GTF3C4.

This fourth use of the invention is preferably in the cosmetics field. This use can also allow the manufacture of a medicament for therapeutic action in the pigmentation field.

In a particular envisaged case, the fourth use described involves a genetic modification whether or not it is induced by an expression product of a DNA fragment as described.

In the case in which gene activation is responsible for the depigmentation, said fourth use of the invention can abolish the function of said gene by introducing an antisense RNA which will block translation of said gene by binding itself to the transcript synthesized by the cell.

In the case in which inactivation of a gene is responsible for depigmentation, said fourth use of the invention can restore the function of said gene by introducing RNA allowing synthesis of the protein encoded by the gene, or the protein encoded by the gene, into the cell or a molecule.

For the four types of uses described above in the context of the invention, the cosmetic uses are preferably in the pigmentation field.

For the four types of uses described above, the product of the invention could be incorporated into a cosmetic or pharmaceutical composition. Said composition comprises, in a pharmaceutically or cosmetically acceptable medium, a quantity of products of the invention in the range 0.001% to 10% by weight per volume.

The composition can be administered orally or applied to the skin (onto any skin zone of the body) and/or onto the scalp or hair.

For oral administration, the composition may contain the product(s) of the invention in solution in a food quality liquid such as an aqueous or hydroalcoholic solution, which may be flavored. They may also be incorporated into a solid ingestable excipient and, for example, be in the form of granules, pills, tablets or dragees. They can also be taken up into solution in a food quality liquid which may then be packaged into ingestable capsules.

Depending on the manner of administration, the composition can be presented in any of the normal galenical forms, particular those in cosmetology.

A preferred composition of the invention is a cosmetic composition adapted for topical application to the scalp and/or the skin.

For topical application, the composition which can be used can in particular be in the form of an aqueous, hydroalcoholic or oily solution or a lotion or serum type dispersion, or as a milk type emulsion with a liquid or semi-liquid consistency obtained by dispersing an oily phase in an aqueous phase (O/W) or vice versa (W/O), or suspensions or emulsions of a soft cream-like consistency or an aqueous or anhydrous gel, or microcapsules or micro particles, or vesicular ionic and/or non ionic dispersions. They can be in the form of an unguent, tincture, cream, pomade, powder, patch, impregnated pad, solution, emulsion or vesicular dispersion, lotion, gel, spray, suspension, shampoo, aerosol or foam. They may be anhydrous or aqueous. They may also consist of solid preparations constituting soaps or cleansing bars.

These compositions are prepared using the usual methods.

In particular, the composition can be a hair care composition, especially a shampoo, a setting lotion, a treatment lotion, a styling cream or gel, a coloring composition (in particular oxidizing dyes) which may be in the form of coloring shampoo, hair restructuring lotions or masks.

When the invention consists in a use for cosmetic applications, the composition is preferably a cream, a hair lotion, shampoo or conditioner.

The qualities of different constituents of the compositions are those conventionally used in the fields under consideration.

When the composition is an emulsion, the proportion of the oily phase can be from 5% to 80% by weight, preferably 5% to 50% by weight with respect to the total composition weight. The oils, waxes, emulsifiers and co-emulsifiers used in the composition in the form of an emulsion are selected from those conventionally used in the cosmetics field. The emulsifier and co-emulsifier are present in the composition in a proportion of 0.3% to 30% by weight, preferably 0.5% to 20% by weight with respect to the total composition weight. The emulsion may also contain lipid vesicles.

When the composition is an oily solution or gel, the oily phase can represent more than 90% of the total composition weight.

In a variation, the composition will be such that the products of the invention are encapsulated in an envelope such as microspheres, nanospheres, oleosomes or nanocapsules, the envelope being selected depending on the chemical nature of the product of the invention.

As an example, the microspheres may be prepared using the method described in European patent application EP-A-0 375 520.

The nanospheres may be in the form of an aqueous suspension and may be prepared using the methods described in French patent applications FR-A-0015686 and FR-A-0101438.

Oleosomes consist of an oil-in-water emulsion formed by oil globules provided with a lamellar liquid crystal envelope dispersed in an aqueous phase (see European patent applications EP-A-0 641 557 and EP-A-0 705 593).

The products of the invention can also be encapsulated into nanocapsules consisting of a lamellar envelope obtained from a silicone surfactant (see EP-A-0 780 115); the nanocapsules can also be prepared from hydrodispersible sulfonic polyesters (see FR-A-0113337).

The products of the invention may also be complexed onto the surface of oily cationic globules regardless of their size (see EP-A-1 010 413, EP-A-1 010 414, EP-A-1 010 415, EP-A-1010416, EP-A-1 013 338, EP-A-1 016 453, EP-A-1 018 363, EP-A-1 020 219, EP-A-1 025 898, EP-A-1 020 101, EP-A-1 120 102, EP-A-1 129 684, EP-A-1 160 005 and EP-A-1 172 077).

Finally, the products of the invention can be complexed onto the surface of nanocapsules or nanoparticles provided with a lamellar envelope (see EP-A-0 447 318 and EP-A-0 557 489) and containing a cationic surfactant on the surface (see the references cited above for cationic surfactants).

In particular, a composition in which the products of the invention have an envelope with a diameter of 10 μm or less is preferred.

In known manner, the composition can also contain the usual adjuvants in the cosmetics field, such as hydrophilic or lipophilic gelling agents, hydrophilic or lipophilic additives, preservatives, antioxidants, solvents, fragrances, fillers, filters, odor absorbers and coloring materials. The quantities of the various adjuvants are those which are conventionally used in the cosmetics field, for example 0.01% to 10% of the total composition weight. Depending on their nature, said adjuvants can be introduced into the oily phase, into the aqueous phase and/or into the lipid spherules.

Oils or waxes that can be cited are mineral oils (Vaseline oil), vegetable oils (the liquid fraction of shea butter, sunflower seed oil), animal oils (perhydrosqualene), synthesis oils (purcellin oil), silicone oils or waxes (cyclomethicone) and fluorinated oils (perfluoropolyethers), beeswax, carnauba wax or paraffin. Fatty alcohols and fatty acids (stearic acid) can be added to said oils.

Suitable emulsifying agents which can be cited are glycerol stearate, polysorbate 60 and the PEG-6/PEG-32/glycol stearate mixture sold by Gattefosse under the trade name Tefose® 63.

Suitable solvents which can be cited are lower alcohols, in particular ethanol and isopropanol and propylene glycol.

Hydrophilic gelling agents which can be used which may be cited are carboxyvinyl polymers (carbomers), acrylic copolymers such as acrylate/alkylacrylate copolymers, polyacrylamides, polysaccharides such as hydroxypropylcellulose, natural gums and clays, and, as lipophilic gelling agents, modified clays such as bentonites, metallic salts of fatty acids such as aluminum stearates and hydrophobic silica, ethylcellulose and polyethylene.

The compositions can have other active agents associated with the product of the invention. Examples of such active agents which can be cited by way of example are:
a agents modulating differentiation and/or proliferation and/or pigmentation of cells of the skin such as retinol and its esters, vitamin D and its derivatives, estrogens such as estradiol, AMPc modulators such as POMC derivatives, adenosine or forskoline and its derivatives, prostaglandins and their derivatives, triiodotrionine and its derivatives;
plant extracts such as those from Iridicaeae or soya, which may or may not contain isoflavones;
micro-organism extracts;
free radical scavengers such as α-tocopherol or its esters, superoxide dismutates or its mimetics, certain metal chelating agents or ascorbic acid and its esters;
anti-seborrheics such as certain sulfur-containing amino acids, 13-cis-retinoic acid, cyproterone acetate;
other agents combating desquamative conditions of the scalp, such as zinc pyrithione, selenium disulfide, climbazole, undecylenic acid, ketoconazole, piroctone olamine (octopirox) or ciclopiroctone (ciclopirox);

In particular, they may be active agents that can stimulate regrowth and/or inhibit hair loss; particular non limiting examples thereof are as follows:
a nicotinic acid esters, in particular tocopherol nicotinate, benzyl nicotinate and C1-C6 alkyl nicotinates such as methyl or hexyl nicotinate;
pyrimidine derivatives such as 2,4-diamino-6-piperidinopyrimidine 3-oxide or “Minoxidil” described in U.S. Pat. No. 4,139,619 and U.S. Pat. No. 4,596,812;
lipoxygenase inhibitors or cyclooxidase inducers encouraging hair regrowth, such as those described by the Applicant in European patent application EP-A-0 648 488;
antibacterial agents such as macrolides, pyranosides and tetracyclins, in particular erythromycin;
calcium antagonist agents such as cinnarizine, nimodipine and nifedipine;
hormones such as estriol or its analogues, or thyroxin and its salts;
antiandrogenous agents such as oxendolone, spironolactone or flutamide;
steroidal or non-steroidal inhibitors of 5-α-reductases, such as those described by the Applicant in European patent applications EP-A-0 964 852 and EP-A-1 068 858, or finasteride;
agonists for ATP-dependent potassium channels, such as cromakalim or nicorandil.

In another implementational possibility, the present invention concerns methods for diagnosing a predisposition to premature canities in an individual.

Premature canities is a phenotype which has been defined by the inventors as, inter alia, being characterized by the appearance of the first white hairs early in life, and preferably at about 18 years of age. Since this phenotype is transmitted to the next generation, it may prove important for individuals for whom one parent or relative is affected, to determine, before the appearance of symptoms, whether or not they will be affected. The diagnostic method of the invention is perfectly suited to individuals under 18 years of age.

Since it is probable that environmental factors play a role in the “canities” phenotype as in “premature canities”, thanks to the methods of the invention, we can determine the risks of developing such a phenotype, i.e. a predisposition to premature canities.

A method of the invention for determining a predisposition to premature canities comprises a first step for selecting one or more markers which will be used in the subsequent steps. The term “marker” means a DNA sequence the various allelic variations of which carry information. Such a marker may be a short sequence of a gene the mutation of which is the source of the phenotype. It may also be a marker which is physically located on the chromosome in a region very close to a gene involved in premature canities. Preferably, the marker is a SNP (single nucleotide polymorphism).

In accordance with a first aspect of a method of the invention, the selected marker or markers belong to the region on human chromosome 6 comprising the HLAG, NT_{—}007592.445, NT_{—}007592.446, NT_{—}007592.506, NT 007592.507, NT_{—}007592.508, HSPA1B, G8, NEU1, NG22, BAT8, HLA DMB, HLA-DMA, BRD2, HLA-DQA1, HLA-DQA2, NT_{—}007592.588, GRM4, RNF23, FLJ22638, NT_{—}007592.459 and NT_{—}007592.457 genes. Preferably, the selected markers belong to the sequence for one of said genes. Preferred genes are the HLAG, NT_{—}007592.445, NT_{—}007592.446, NT_{—}007592.506, NT_{—}007592.507, NT 007592.508, HSPA1B, G8, NEU1, NG22, BAT8, HI-A-DMB, HLA-DMA, BRD2, HLA-DQA1, HLA-DQA2 and NT_{—}007592.588 genes, more particularly the NT_{—}007592.506, NT_{—}007592.507 and NT_{—}007592.508 genes.

In accordance with a second aspect of a method of the invention, the selected markers belong to the region on human chromosome 9 comprising the FREQ, NT_{—}030046.18, NT_{—}030046.17, GTF3C5, CEL, CELL, FS, ABO, BARHL1, DDX31, GTF3C4 and Q96MA6 genes. Preferably, the selected markers belong to the sequence for one of said genes. Preferred genes are the BARHL1, DDX31, GTF3C4 and Q96MA6 genes, more particularly the DDX31 and GTF3C4 genes.

The next step in carrying out the method of the invention consists, for the selected marker or markers, in determining the alleles present in a sample of genetic material from the individual undergoing the diagnostic test. Two different alleles carried by the two versions of the chromosome can be identified.

The sample containing the genetic material may be blood, a single drop being sufficient to carry out the method of the invention. Other body fluid samples may be used in the context of the invention. It is also possible to use a few cells from the individual. The skilled person will known how to determine which sample could be used in the context of this test, while minimizing discomfort for the individual concerned. This diagnostic test could optionally be coupled with other genetic tests.

Routine techniques which are well known to the molecular biologists could be used to determine alleles for the selected marker or markers; in particular, hybridization tests are particularly appropriate in this type of step.

Various markers are potentially preferred in the context of carrying out the method of the invention. In particular, bi-allelic markers may prove particularly suitable if one allelic form translates as a predisposition to premature canities while the other allelic form, in contrast, reflects the absence of said predisposition. Other more routine markers are polymorphic and can be found in at least two allelic forms, and generally more than two.

Markers which can be selected for the first step of the method of the invention include SNPs which are particularly well known. When selecting the marker, it is very important to base it on the informative value of the marker polymorphism. One particularly advantageous situation consists of selecting a marker certain allelic variations of which translate into a predisposition to premature canities while all of the other variations reflect the absence of said predisposition. In many situations, the marker does not entirely satisfy said condition, i.e. certain alleles are preferentially present but not exclusively present in individuals predisposed to canities. In these situations, it may be judicious to select several markers to establish a diagnostic test which is as reliable as possible.

When the selected markers are SNPs, the various allelic variations correspond to modification of a base. A particularly advantageous situation to be investigated when selecting a marker corresponds to the situation in which certain alleles (modification of a base) are characteristic of a predisposition to premature canities.

In accordance with the first aspect of a method of the invention, the marker or markers selected for the first step could be selected from the following SNP markers: 2734988, 2734967, 1419664, 2517502, rs733539, rs494620, 154973, 206779, 60071, rs763028.

In accordance with the second aspect of the method of the invention, the marker or markers selected for the first step can be selected from the following SNP markers: 418620, rs302919, 913705, 932886, 429269 and rs2526008.

The methods of the invention are not limited to the two steps described and may contain other steps anterior or posterior to the two steps mentioned.

In particular, a method of the invention may comprise the supplemental step of comparing the allelic form of the selected marker or markers with the allelic form of the marker or markers in other individuals. This supplemental comparison step may prove necessary in order to establish a diagnosis. In this case, it may be useful to make a comparison with the form of the marker or markers in individuals manifestly affected with premature canities and optionally also with the form of the marker or markers in individuals who are manifestly free of said predisposition.

Given that premature canities is probably a multigenic disorder, the causes of predisposition are many and it may be difficult for all of them to be envisaged exhaustively. In contrast, within one family some members of which are prematurely affected with canities, it is highly probable that the cause of the susceptibility is unique. For this reason, during the comparison step, mentioned as an optional third step in the methods of the invention, a particularly information-rich comparison is comparison of alleles of the marker for the individual undergoing the test with alleles of the same marker for persons in his family where the phenotype is known. If several markers have been selected, this operation should preferably be repeated for all markers.

The present invention also concerns methods for screening molecules having a particular effect. In particular, the invention concerns a method for identifying molecules that can modulate the function of a polynucleotide fragment.

In accordance with the first aspect of the invention, the polynucleotide fragment the function of which is to be modulated comprises at least 18 consecutive nucleotides the sequence for which corresponds to all or part of a gene selected from 22 genes of the invention on human chromosome 6. Preferred genes are the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT 007592.588 genes, more particularly the NT_{—}007592.506, NT_{—}007592.507 and NT_{—}007592.508 genes.

In accordance with the second aspect of the invention, the polynucleotide fragment the function of which is to be modulated comprises at least 18 consecutive nucleotides the sequence for which corresponds to all or part of a gene selected from the 12 genes of the invention on chromosome 9. Preferred genes are the BARHL1, DDX31, GTF3C4 and Q96MA6 genes, more preferably the DDX31 and GTF3C4 genes.

The method for identifying molecules that are capable of modulating the function of one or other of said fragments comprises a step for bringing the molecule to be tested into the presence of the polynucleotide fragment. Another step in the method is detecting a variation in a parameter linked to the function of said fragment, for example detecting any binding of said molecule to the polynucleotide fragment demonstrated by a ligand detection technique.

The “second use of the invention” is the use of an agent modulating the function of a DNA fragment corresponding at least in part to one of the genes of the invention. The screening method allows such agents to be identified.

The various functions which a polynucleotide fragment can carry out have already been explained in the present application. In particular, these functions depend on the nature of the polynucleotide fragment, for example whether it is DNA or RNA. Modulation of the function may correspond to a reduction in the capacity to be transcribed or translated or to a change in the capacity to interact with other factors. Depending on the properties of said fragment, the skilled person can determine the parameter the variation of which is easy to monitor.

The identification method of the invention is not limited to the steps described above; other anterior or posterior steps can be applied.

The present invention also encompasses molecules identified by the method described above. In particular, the present invention encompasses inhibitors for the functions of the polynucleotide fragments of the invention.

The present invention also concerns methods for screening molecules that are capable of modulating the function of the expression product of a polynucleotide fragment of the invention.

In accordance with the first aspect of the invention, the expression product the function of which is to be modulated is that of a DNA fragment that belongs to and/or corresponds to all or part of a gene selected from the 22 genes of the invention on human chromosome 6. Preferred genes are the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588 genes, more particularly the NT_{—}007592-506, NT_{—}007592.507 and NT_{—}007592.508 genes.

In accordance with the second aspect of the invention, the expression product the function of which is to be modulated is that of a DNA fragment belonging to and/or corresponding to all or part of a gene selected from the 12 genes of the invention on chromosome 9. Preferred genes are the BARHL1, DDX31, GTF3C4 and Q96MA6 genes, more particularly the DDX31 and GTF3C4 genes.

Preferably, the DNA fragment comprises at least 18 nucleotides.

The method for identifying molecules that can modulate the function of the expression product of a DNA fragment as described comprises a step for bringing the test molecule into the presence of the expression product. A further step in the method is detecting a variation in a parameter linked to the function of said expression product, for example detecting any binding of said molecule to the expression product, demonstrated by a ligand detection method.

As mentioned above in the application, the term “expression product of a DNA fragment” means both RNA molecules derived from transcription of the fragment at any maturation stage, and polypeptides from translation, at any maturation stage. For a molecule of RNA, different maturation stages are represented for example by the presence or absence of a cap, a polyadenylated tail. The term “various maturation stages of a polypeptide” includes, inter alia, polypeptides before and after folding, before and after cleavage of the various addressing signals, with or without glycosylation, and with or without disulfide bridges.

The functions fulfilled by the expression products of DNA fragments are very numerous and depend on the nature of the expression product in question. Examples have already been given above in the present application.

The “third use of the invention” is the use of an agent modulating the function of the expression product of a DNA fragment. Screening method allows such agents to be identified. Regarding what should be understood by the term “modulate the function of the expression product of a DNA fragment”, examples have already been given to define the third use of the invention.

Depending on the properties of said expression product, the skilled person is capable of determining the parameter the variation of which is easy to monitor.

The identification method of the invention is not limited to the steps described above; other anterior or posterior steps can be applied.

The present invention also encompasses the molecules identified by the method described above. In particular, the present invention encompasses inhibitors of the functions of the expression products of the polynucleotide fragments of the invention.

The present invention also allows to bring to light the mutations in genes involved in skin, hair and phanera pigmentation, for the genes of the invention One particular use envisaged by the present invention thus consists of using SNP markers described above in each of the regions comprising the genes of interest with the aim of localizing mutations in the genes involved in pigmentation more accurately, and more particularly those involved in the progressive or sudden interruption in the pigmentation of the skin or of the phanera.

Within the context of said uses, in accordance with the first aspect of the invention, the markers used to determine the genes of interest are selected from SNP markers: 2734988, 2734967, 1419664, 2517502, rs733539, rs494620, 154973, 206779, 60071, rs763028.

In accordance with the second aspect of the invention, the markers used to determine the genes of interest are selected from SNP markers 418620, rs302919, 913705, 932886, 429269 and rs2526008.

The present invention resides in the identification by the inventors of genes on human chromosomes 6 and 9 involved in the pigmentation or depigmentation phenomenon. This genetic basis has allowed them to envisage the uses in therapy and cosmetics described above, as well as the diagnostic methods illustrated above.

However, as mentioned above, the inventors suspect that many genes are involved in pigmentation phenomena and in those linked to regulation and cessation of that pigmentation. In particular, it is envisaged that the genes of the invention on chromosome 9 are principally responsible for canities, while the genes of the invention on chromosome 6 are concerned with the premature nature of the onset of canities. For this reason, an important part of the present invention consist of combining the results obtained for the genes of the invention, in order to extract the most complementary information.

In particular, a first combinational use in the cosmetics and therapeutic fields preferably employs at least two polynucleotide fragments the sequence of each of which corresponds at least in part to that of a gene selected from the 22 genes of the invention on human chromosome 6 or to that of a gene selected from the 12 genes of the invention on chromosome 9.

Preferred genes on chromosome 6 are the HLAG, NT_{—}007592.445, NT_{—}007592.446, NT_{—}007592.506, NT 007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588 genes, more particularly the NT_{—}007592.506, NT_{—}007592.507 and NT_{—}007592.508 genes.

Preferred genes on chromosome 9 are the BARHL1, DDX31, GTF3C4 and Q96MA6 genes, more particularly the DDX31 and GTF3C4 genes.

Preferably, of the different polynucleotide fragments employed, at least two have sequences corresponding to two distinct genes. It is also envisageable that for the same gene of the invention, the various fragments employed have different chemical natures, for example DNA for the first fragment and RNA for the second fragment. It can also be envisaged that the different fragments could have a sequence corresponding to the same gene, but with small variations permitted by the definition of “corresponding sequences”, i.e. at most one different nucleotide in 10, preferably 1 in 100.

The fragments of the invention contain at least 18 consecutive nucleotides, said 18 nucleotides forming the sequence which must at least partially correspond to one of the genes of the invention.

All of the preferred uses, the chemical nature of the fragments, their environment, have already been explained in detail in the section describing the first use of the invention.

When at least two polynucleotide fragments are used, said two fragments are preferably carried by distinct molecules. It can also be envisaged that said two fragments could, for example, form part of the same vector. In a preferred case, the different fragments are of the same chemical nature, for example DNA for all fragments.

For therapeutic uses, the polynucleotide fragments are used in the manufacture of a medicament.

A second combinational use envisaged by the present invention in the cosmetics and therapeutic fields is the use of a combination of at least two agents each modulating the function of a DNA fragment selected from fragments belonging and/or corresponding to all or part of one of the 34 genes of the invention on human chromosomes 6 and 9.

Preferred genes on chromosome 6 are the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588 genes, more particularly the NT_{—}007592.506, NT_{—}007592.507 and NT_{—}007592.508 genes.

Preferred genes on chromosome 9 are the BARHL1, DDX31, GTF3C4 and Q96MA6 genes, more particularly the DDX31 and GTF3C4 genes.

Preferably, of the different agents employed, at least two modulate the function of DNA fragments corresponding to two distinct genes. It is also envisageable that for the same gene of the invention, the different agents employed modulate different functions of the same DNA fragment.

The fragments of DNA the function of which is modulated in accordance with the invention preferably contain at least 18 consecutive nucleotides, said 18 nucleotides forming the sequence which must at least partially correspond to one of the genes identified in the context of the invention.

All of the preferred uses for said agents have already been explained in detail in the section describing the second use of the invention.

For therapeutic uses, the agents are used in the manufacture of a medicament.

A third combinational use envisaged by the present invention in the cosmetics and therapeutic fields is the use of a combination of at least two agents each modulating the function of an expression product of a DNA fragment selected from fragments belonging and/or corresponding to all or part of one of the 34 genes of the invention on human chromosomes 6 and 9.

Preferred genes on chromosome 6 are the HLAG, NT_{—}007592.445, NT_{—}007592.446, NT 007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588 genes, more particularly the NT 007592.506, NT_{—}007592.507 and NT_{—}007592.508 genes.

Preferred genes on chromosome 9 are the BARHL1, DDX31, GTF3C4 and Q96MA6 genes, more particularly the DDX31 and GTF3C4 genes.

Preferably, of the different agents employed, at least two modulate the function of the expression product of DNA fragments corresponding to two distinct genes. It is also envisageable that for the same gene of the invention, the different agents employed could modulate different functions of the same expression product of a DNA fragment, for example RNA at different maturation stages, or RNA from different splices.

The fragments of DNA the function of the expression product of which is modulated in accordance with the invention preferably contain at least 18 consecutive nucleotides, said 18 nucleotides forming the sequence which must at least partially correspond to one of the genes of the invention.

All of the preferred uses of said agents have already been explained in detail in the section describing the first use of the invention.

For therapeutic uses, the polynucleotide fragments are used in the manufacture of a medicament.

A fourth combinational use envisaged by the present invention in the cosmetics and therapeutic fields is the use of a combination of at least two expression products of DNA fragments selected from fragments belonging and/or corresponding to all or part of one of the 34 genes of the invention on human chromosomes 6 and 9.

Preferred genes on chromosome 6 are the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588 genes, more particularly the NT_{—}007592.506, NT_{—}007592.507 and NT_{—}007592.508 genes.

Preferred genes on chromosome 9 are the BARHL1, DDX31, GTF3C4 and Q96MA6 genes, more particularly the DDX31 and GTF3C4 genes.

Preferably, of the different expression products employed, at least two are derived from DNA fragments corresponding to two distinct genes. It is also envisageable that for the same gene of the invention, the different expression products employed could derive from the same DNA fragment, for example from RNA at different maturation stages, or RNA from different splices The same possibilities apply to polypeptides derived from RNA translation.

The fragments of DNA the function of the expression product of which is modulated in accordance with the invention preferably contain at least 18 consecutive nucleotides, said 18 nucleotides forming the sequence which must at least partially correspond to one of the genes of the invention.

All of the preferred uses of said agents have already been explained in detail in the section describing the first use of the invention.

For therapeutic uses, the polynucleotide fragments are used in the manufacture of a medicament.

For the four types of combinational uses described above in the context of the invention, the cosmetic uses preferably apply to the pigmentation field.

Of the many combinations envisaged by the present invention, a highly advantageous combination comprises at least one polynucleotide fragment corresponding to all or part of a gene on human chromosome 6 selected from the HLAG, NT_{—}007592.445, NT_{—}007592.446, NT_{—}007592.506, NT 007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes for the first combinational use.

For the second combinational use, of the agents employed, at least one of said agents preferably modulates the function of a DNA fragment corresponding to or belonging to a gene on human chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes.

For the third combinational use, of the agents employed, at least one of said agents preferably modulates the function of the expression product of a DNA fragment corresponding to or belonging to a gene on human chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes.

For the fourth combinational use, of the expression products employed, at least one of said expression products preferably derives from a DNA fragment corresponding to or belonging to a gene on human chromosome 6 selected from the HLAG, NT_{—}007592.445, NT_{—}007592.446, NT_{—}007592-506, NT_{—}007592.507, NT_{—}007592.508, HSPA1B, G8, NEU1, NG22, BAT8, HLA-DMB, HLA-DMA, BRD2, HLA-DQA1, HLA-DQA2, NT_{—}007592.588, GRM4, RNF23, FLJ22638, NT_{—}007592.459 and NT_{—}007592.457 genes.

In all of the combinational uses described above, in cosmetics or in therapeutics, a plurality of fragments or for expression products or a plurality of agents modulating the function of fragments or a plurality of agents modulating the function of the following expression product are used, in which the fragment in question corresponds to all or part of one of the genes of the invention. Preferably, at least one of the fragments in question corresponds to a gene on human chromosome 6 selected from the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2, NT 007592.588, GRM4, RNF23, FLJ22638, NT_{—}007592.459 and NT_{—}007592.457 genes.

Preferably, at least one of the fragments in question in the combinational uses corresponds to a gene on human chromosome 9 selected from the FREQ, NT_{—}030046.18, NT_{—}030046.17, GTF3C5, CEL, CELL, FS, ABO, BARHL1, DDX31, GTF3C4 and Q96MA6 genes.

For the four types of combinational use described above, the combinations of the invention could be incorporated into a cosmetic or pharmaceutical composition as described above.

In order to profit from the combination of said genes on the two chromosomal regions, the present invention also concerns combinational methods. Said methods are employed to determine any predisposition to premature canities.

A combinational method of the invention for determining a predisposition to premature canities comprises a first step for selecting at least two markers which will be employed in subsequent steps. The selected markers are selected from markers belonging to regions of human chromosomes 6 and 9 comprising the 34 genes of the invention. Preferably, the selected markers belong to the sequence for one of the genes mentioned on chromosomes 6 and 9. Preferred genes on chromosome 6 are the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588 genes, more particularly the NT_{—}007592.506, NT_{—}007592.507 and NT_{—}007592.508 genes.

Preferred genes on chromosome 9 are the BARHL1, DDX31, GTF3C4 and Q96MA6 genes, more particularly the DDX31 and GTF3C4 genes.

Preferably, among the selected markers, at least two do not belong to the same gene of the invention. In a particular case of the present invention, the markers, a minimum of two in number, are selected from the following list of SNP markers: 2734988, 2734967, 1419664, 2517502, rs733539, rs494620, 154973, 206779, 60071, rs763028, 418620, rs302919, 913705, 932886, 429269 and rs2526008.

The subsequent step in carrying out the method of the invention consists, for the selected markers, in determining the alleles present in a sample of genetic material from the individual undergoing the diagnostic test. Two different alleles carried by the two versions of the chromosome can be identified.

The conditions for carrying out said methods have already been explained in the part concerning diagnostic methods.

The combinational methods of the invention are not limited to the use of two markers, nor are they limited to the two steps described; they may contain other steps which are anterior or posterior to the two steps already mentioned.

In particular, a combinational method of the invention may comprise the supplementary step of comparing the allelic form of the selected markers with the allelic form of the same markers in other individuals. This supplementary comparison step may prove necessary in order to establish a diagnosis. In this case, it may be useful to make the comparison with the form of the markers in individuals who are manifestly affected by premature canities and optionally also with the form of the markers in individuals who are manifestly free from such a predisposition.

As was the case for the diagnostic methods already mentioned in the present application, a particularly advantageous situation consists of comparing the alleles of the selected markers with alleles of those same markers in other individuals from the same family as the individual to be diagnosed.

Further, in another patent application filed on the same day by the same applicant, the inventors have also used a similar method to bring to light other genomic regions on human chromosomes 3, 5 and 11 also involved in the pigmentation or depigmentation phenomenon (see also Examples 1 and 3 of the present application). Those regions are delimited on chromosome 3 by the microsatellite markers D3S1277 and D3S1285, on chromosome 5 by the microsatellite markers D5S2115 and D5S422 and on chromosome 11 by the microsatellite markers D11S898 and D11S925. More particularly, the inventors have identified the genes in those regions which are statistically involved in premature canities in certain subjects, namely the genes KIAA1042, CCK, CACNA1D, ARHGEF3 and AL133097 on chromosome 3, the genes KLHL3, HNRPA0, CDC25C, EGR1, C5orf6, C5orf7, LOC51308, ETF1, HSPA9B, PCDHA1 to PCDHA13, CSF1R, RPL7, PDGFRB, TCOF1, AL133039, CD74, RPS14, NDST1, G3BP, GLRA1, C5orf3, MFAP3, GALNT10 and FLJ117151 on chromosome 5 and the genes GUCY1A2, CUL5, ACAT1, NPAT, ATM, AF035326, AF035327, AF035328, BC029536, FLJ20535, DRD2, ENS303941, IGSF4, LOC51092, BC010946, TAGLN, PCSK7 and ENS300650 on chromosome 11. This genetic base has allowed the inventors to envisage therapeutic and cosmetic uses similar to those described above, and diagnostic methods that are similar to those illustrated above.

In the context of the present invention, it is thus advantageous to exploit these results by combining the products produced by taking into account sequences involved in canities on chromosomes 6 and 9, and the products produced by taking into account sequences involved in canities on chromosomes 3, 5 and 11.

A particularly preferred combination in the context of the present invention is the combination of products produced by taking into account sequences of interest on chromosome 9 and the products produced taking into account the sequences of interest on chromosomes 6, 11, 5 and 3.

In particular, a first use in the cosmetics and therapeutic field preferably employs at least two polynucleotide fragments the sequence of the first of which corresponds at least in part to that of the DDX31 or GTF3C4 on chromosome 9 and the sequence of at least one other corresponds at least in part to one of the following genes: KIAA1042, CCK, CACNA1D, ARHGEF3 and AL133097 on chromosome 3, KLHL3, HNRPA0, CDC25C, EGR1, C5orf6, C5orf7, LOC51308, ETF1, HSPA9B, PCDHA1 to PCDHA13, CSF1R, RPL7, PDGFRB, TCOF1, AL133039, CD74, RPS14, NDST1, G3BP, GLRA1, C5orf3, MFAP3, GALNT10 and FLJ117151 on chromosome 5 and GUCY1A2, CUL5, ACAT1, NPAT, ATM, AF035326, AF035327, AF035328, BC029536, FLJ20535, DRD2, ENS303941, IGSF4, LOC51092, BC010946, TAGLN, PCSK7 and ENS300650 on chromosome 11.

A second use envisaged by the present invention in the cosmetics and therapeutic field is the use of a combination of at least two agents each modulating the function of a DNA fragment, the first fragment belonging to and/or corresponding to all or part of the DDX31 or GTF3C4 gene on chromosome 9, at least one other fragment belonging to and/or corresponding to all or part of the following genes: KIAA1042, CCK, CACNA1D, ARHGEF3 and AL133097 on chromosome 3, KLHL3, HNRPA0, CDC25C, EGR1, C5orf6, C5orf7, LOC51308, ETF1, HSPA9B, PCDHA1 to PCDHA13, CSF1R, RPL7, PDGFRB, TCOF1, AL133039, CD74, RPS14, NDST1, G3BP, GLRA1, C5orf3, MFAP3, GALNT10 and FLJ17151 on chromosome 5 and GUCY1A2, CUL5, ACAT1, NPAT, ATM, AF035326, AF035327, AF035328, BC029536, FLJ20535, DRD2, ENS303941, IGSF4, LOC51092, BC010946, TAGLN, PCSK7 and ENS300650 on chromosome 11.

A third use envisaged by the present invention in the cosmetics and therapeutic field is the use of a combination of at least two agents each modulating the function of the expression product of a DNA fragment, the first fragment belonging to and/or corresponding to all or part of the DDX31 or GTF3C4 gene on chromosome 9, at least one other fragment belonging to and/or corresponding to all or part of the following genes: KIAA1042, CCK, CACNA1D, ARHGEF3 and AL133097 on chromosome 3, KLHL3, HNRPA0, CDC25C, EGR1, C5orf6, C5orf7, LOC51308, ETF1, HSPA9B, PCDHA1 to PCDHA13, CSF1R, RPL7, PDGFRB, TCOF1, AL133039, CD74, RPS14, NDST1, G3BP, GLRA1, C5orf3, MFAP3, GALNT10 and FLJ117151 on chromosome 5 and GUCY1A2, CUL5, ACAT1, NPAT, ATM, AF035326, AF035327, AF035328, BC029536, FLJ20535, DRD2, ENS303941, IGSF4, LOC51092, BC010946, TAGLN, PCSK7 and ENS300650 on chromosome 11.

A fourth use envisaged by the present invention in the cosmetics and therapeutic field is the use of a combination of at least two expression products of DNA fragments, the first fragment belonging to and/or corresponding to all or part of the DDX31 or GTF3C4 gene on chromosome 9, at least one other fragment belonging to and/or corresponding to all or part of the following genes: KIAA1042, CCK, CACNA1D, ARHGEF3 and AL133097 on chromosome 3, KLHL3, HNRPA0, CDC25C, EGR1, C5orf6, C5orf7, LOC51308, ETF1, HSPA9B, PCDHA1 to PCDHA13, CSF1R, RPL7, PDGFRB, TCOF1, AL133039, CD74, RPS14, NDST1, G3BP, GLRA1, C5orf3, MFAP3, GALNT10 and FLJ117151 on chromosome 5 and GUCY1A2, CUL5, ACAT1, NPAT, ATM, AF035326, AF035327, AF035328, BC029536, FLJ20535, DRD2, ENS303941, IGSF4, LOC51092, BC010946, TAGLN, PCSK7 and ENS300650 on chromosome 11.

Finally, the present invention concerns a kit comprising a combination of at least two polynucleotide fragments selected from those comprising at least 18 consecutive nucleotides the sequence of which corresponds to all or part of one of the 34 genes of the invention on human chromosomes 6 and 9.

Preferred genes on chromosome 6 are the HLAG, NT_{—}007592.445, NT_{—}007592.446, NT_{—}007592.506, NT_{—}007592.507, NT_{—}007592.508, HSPA1B, G8, NEU1, NG22, BAT8, HLA-DMB, HLA-DMA, BRD2, HLA-DQA1, HLA-DQA2 and NT_{—}007592.588 genes, more particularly the NT_{—}007592.506, NT_{—}007592.507 and NT_{—}007592.508 genes.

Preferred genes on chromosome 9 are the BARHL1, DDX31, GTF3C4 and Q96MA6 genes, more particularly the DDX31 and GTF3C4 genes.

The fragment length is preferably in the range 15 to 5000 nucleotides, more preferably in the range 30 to 5000, and still more preferably in the range 40 to 3000 nucleotides.

### LEGEND TO FIGURES

FIG. 1 Composition of families analyzed for region-candidate linkage

FIG. 2 Candidate region for PC on chromosome 6 (FIG. 2A) and on chromosome 3 (FIG. 2B): chromosomal localization and marker distribution

FIG. 3 Graph of NPL scores obtained for global genome multipoint non parametric linkage analysis on PC families for chromosomes 6, 9 (FIG. 3A), 3, 5 (FIG. 3B) and 11 (FIG. 3C)
abscissa=position on genetic map (0=pter)
ordinate=NPL score

FIG. 4 Diagram of PC loci identified on chromosomes 6 and 9 by global genome study. The distance between markers is indicated in cM.

FIG. 4A: chromosome 6, locus 6p21-p12

FIG. 4B: chromosome 9, locus 9q34

FIG. 4C: chromosome 11, locus 11q14-q22

FIG. 4D: chromosome 5, locus 5q31-q32

FIG. 4E: chromosome 3, locus 3p14.1-p12.3

FIG. 5 Simulated Lod scores for the 29 selected families.
The columns show the mean Lod score, standard deviation, the minimum Lod score, the maximum Lod score and the group (A-E) in which the family is placed according to the score.

FIG. 6 Potential Lod scores by family as a function of the degree of genetic heterogeneity of PC.

FIG. 7 Detailed Lod scores by family as a function of the degree of genetic heterogeneity of PC: new simulation after including new families

FIG. 8 New simulation for final families to investigate candidate chromosomal regions. Potential Lod scores as a function of the degree of heterogeneity.
Results expressed by family

FIG. 9 Probability (%) of achieving or exceeding a Lod score of 1, 2 or 3 for each degree of heterogeneity.
Results expressed by family.

FIG. 10 Comparison of composition of families between candidate region analysis and global genome analysis.

FIG. 11 Potential Lod scores as a function of the degree of heterogeneity of PC.
Results expressed by family.

FIG. 12 Probability (%) of achieving or exceeding a Lod score of 1, 2 or 3 for each degree of heterogeneity.
Results expressed by family.

FIG. 13 Summary diagram of the different steps in analyzing regions A and B using techniques based on SNPs.

FIG. 14 Composition of 4 pools. Pools AI and AII are composed of individuals with premature canities. The two control pools BI and BII are composed of individuals “crossed” for age and origin with individuals with premature canities.

FIG. 15 Graph indicating the significance of 288 SNPs tested on pools for regions A. The SNPs are along the abscissa, numbered 1 to 288 along region A (from telomere p to telomere q), each SNP being separated from its neighbors by an average 30 kb region. The value l/p is up the ordinate, p being the statistical significance. However, l/p values of more than 500 (i.e. p<0.02) are maximized to 500.

FIG. 16 Graph indicating the significance of 171 SNPs tested on pools for regions B. The SNPs are along the abscissa, numbered 1 to 171 along region B (from telomere p to telomere q), each SNP being separated from its neighbors by an average 30 kb region. The value l/p is up the ordinate, p being the statistical significance. However, l/p values of more than 500 (i.e. p<0.02) are maximized to 500.

FIG. 17 Table showing the 43 SNPs retained for individual genotyping. The first column shows their number (number assigned in preceding step from 1 to 288 along region A from telomere p to telomere q). The second column shows the name of the SNP. The subsequent columns show the values for the different A-B comparisons (AI-BI; AII-BII; AI-BII) with the associated p value. The note “M” indicates a value for “p” of less than 0.05. The last column indicates the gene which may be overlapped by said SNP.

FIG. 18 Table showing the 33 SNPs retained for individual genotyping. The columns contain the same type of information as for FIG. 17.

FIG. 19 Table showing the 43 SNPs retained for individual genotyping. The first column shows the position on the chromosome, the second column shows their identifier, the next column gives their number (assigned in the preceding step from 1 to 288 along region A from telomere p to telomere q). The subsequent columns indicate whether the SNPs are present in a cluster or as double spots.

FIG. 20 Table showing the 33 SNPs retained for individual genotyping. The columns contain the same types of information as for FIG. 19.

FIG. 21 Diagram of the results of linkage disequilibrium on region A. The significance of associations between SNPs taken in pairs is shown by a color coding.

FIG. 22 Diagram of the results of linkage disequilibrium on region B. The significance of associations between SNPs taken in pairs is shown by a color coding.

FIG. 23 Graph comparing allele/genotype frequencies for each SNP of region A in the “premature canities” and control groups, highlighting SNP/phenotype associations. The genes concerned are shown along the abscissa with the SNPs.

FIG. 24 Graph comparing allele/genotype frequencies for each SNP of region B in the “premature canities” and control groups, highlighting SNP/phenotype associations. The genes concerned are shown along the abscissa with the SNPs.

### EXAMPLES

### Example 1

### Summary of Studies

In order to localize the gene or genes for premature canities (PC), a segregation analysis (genetic linkage) program was carried out in families for whom this trait is transmitted across the generations. At the end of a series of pre-selections on the basis of the statistical power of the sample and phenotype confirmation, twelve families were retained to participate in a linkage study and DNA was prepared from a sample of peripheral blood from each of the informative individuals (with and without the trait). The study was carried out using two principal approaches, analysis targeted on a candidate region and a global genome study on the twenty-two autosomal chromosomes and the X chromosome.

From the set of analyses carried out, fixing or not fixing the parameters for transmitting the PC trait, two potential loci were discerned on chromosomes 6 and 9. The locus on chromosome 6p21-p12 between the markers D6S1629 and D6S1280 in the region of the gene of the major histocompatibility complex (MHC, HLA) showed robust evidence for containing a predisposition gene and another locus (chromosome 9q32-q32) also showed signs suggesting a link to PC.

Three other loci (chromosomes 11q14-q22, 5q31-q32, 3p14.1-p12.3) also showed signs suggesting a link to PC.

This study, and in particular the discordance between the scores obtained for the parametric/non-parametric analyses, suggests that premature canities is not caused by a small number of genes with major effect, but rather it is governed by a multifactorial system involving the action of several predisposition genes.

### Introduction

The hereditary nature of premature canities (PC) or the appearance of white hairs early in life is a long-held hypothesis because of the familial nature of premature whitening of the hair in some people.

To explore canities from a genetic viewpoint, a DNA segregation study was carried out in families in which canities appeared very early in life. To guarantee the best chances of success for this gene hunt, the composition of the sample for the study was determined using a rigorous protocol for attributing phenotype and for selecting families. The PC phenotype was only attributed to individuals of less than 25 years of age who had white hairs and for whom half of the hair of the head was gray at 30 years old. The families were retained for study on the basis of their statistical performance in the segregation analysis.

This part of the study is described using four principal periods:
A-period 1: Determination of the potential of the study. A first selection of the most informative families was carried out by a linkage analysis simulation.
B-period 2: Medical confirmation of phenotypes and collecting blood samples from pre-selected families. This verification campaign produced a new list of candidate families for the study. A new linkage simulation allowed the potential of the corrected sample to be estimated.
C-period 3: Genetic analysis with candidate chromosomal regions for PC. First phase of DNA analysis for chromosomal regions which could contain PC genes.
D-period 4: Global genetic analysis for PC over the entire human genome. Analysis of familial segregations on DNA from the 22 autosomal chromosomes and the X chromosome to detect regions which link to the PC trait.

The results obtained for each period are shown in the form of tables and Figures in a summary manner in the summarizing tables or in more detail in the detailed tables.

### Results

A-Period 1: Choice of Families with the Aid of Binding Analysis Simulation

At the end of an attempt to select families with premature canities using informativity criteria for gene localization, 29 families underwent a genetic linkage analysis simulation. On the basis of the availability of all individuals, this project appeared to have very encouraging potential for success as nineteen pedigrees (i.e. 255 individuals) were then retained. This conclusion was only valid if the phenotypes were confirmed and if the majority of subjects agreed to participate in the study. In this selection there were seven highly informative families who individually could achieve or exceed a Lod score of Z4.00 (i.e. greater than the lower limit of significance for a Lod score, which is Z=3.00). To stand the greatest chance of success, it was very important that the PC diagnosis was attributed rigorously.

The results of this study allowed the families which were then collected for the genetic study to be determined because of the robust nature of the clinical evaluation.

### 1. Criteria for Attributing a PC Phenotype

Twenty-nine families out of 65 were retained using structural criteria (total number of individuals, those affected, available) for the simulation analysis

During the simulation process:
a) software generated a series of file code replicates by assigning simulated genotypes. The file obtained for each family explored several allelic combinations (genotypes) in each individual;
b) software then analyzed each replicate to estimate the possible Lod scores (Z) for genetic linkage analysis in each family. The results, in the form of minimum, mean and maximum Lod scores, allowed the potential of each family in this type of study to be evaluated.

Clearly, these estimates only remain valid in the case in which each individual was viewed as having been attributed with the correct phenotype. In the event of uncertainty, the phenotype had to be indicated as unknown; it was then not taken into account in the study and thus did not have to be sampled. The Lod score reduced (in varying proportions) each time an individual was removed for an uncertain phenotype.

Genealogical trees were re-drawn using pedigree editing software which also constructed coded files (preplink files) for the genetic linkage analysis. In addition to codes indicating for each individual the parentage, sex, phenotype and genotype which was supplemented by the SLINK simulation software, an availability code (cd) (table 1) was attributed. It also weighted the informative character of each individual in the study using a code from 0 to 3.

The phenotype for each individual was assigned using the information in the pedigrees and descriptive tables of the Genormax report. However, for some individuals, the phenotype was modified using the criteria indicated in Table 2. The individuals not present in the initial pedigrees (identified by a number only) were phenotypically unknown, and were considered to be unavailable (cd=3).

a. Availability Codes

During the simulation, the only individuals taken into account were those for whom (Table 1):
it was possible according to the Genormax study to remove blood to prepare DNA for the genetic study (age, domicile in France/overseas/foreign, consent, a priori);
the phenotype for premature canities had been clearly defined (Table 2).

TABLE 1
Definition of availability codes

| availability code (cd) | DNA | phenotype |
|---|---|---|
| 0 | unavailable | known |
| 1 | available | unknown |
| 2 | available | known |
| 3 | unavailable | unknown |

TABLE 2
Definition or phenotypes

| | <25 years | 25 years | >25 years |
|---|---|---|---|
| no white hairs | 0 | 0 | 1 |
| a few white hairs (qb) | 2 | 0 | 0 |
| (less than 50%) | | | |

b. Assignment of Phenotype According to Age

In order to avoid the risk of errors, the following criteria were defined for assigning phenotype as a function of age in individuals below 30 years of age. However, during the final clinical examination, it was desirable for the definition of the phenotype to be more quantitative.

c. Other Parameters

After examining the variation in the maximum Lod score in the Can65 family (test 100, 200, 300, 500 replications), the number of replications (generations of allelic combinations) was finally fixed at 200.

The frequency of the trait was fixed at 1%. The number of possible alleles for the genotype was fixed at 6 with an equivalent frequency for each one.

### 2. Results

a—Classification of Families According to Scores

Table 3 gives an indication of the GENORMAX family potential as a function of the maximum Lod score (Zmax) achieved (group A-E). FIG. 5 shows the simulated Lod score for each family.

TABLE 3
Families/maximum Lod score statistics.

| maximum Lod score (LMx) | number of families | group |
|---|---|---|
| Zmax ≧ or = 4 | 7 | A |
| 3 ≦ Zmax < 4 | 6 | B |
| 2 ≦ Zmax < 3 | 6 | C |
| 1 ≦ Zmax < 2 | 7 | D |
| Zmax < 1 | 3 | E |

The results shown come from the table in FIG. 5.

The maximum Lod score could only be achieved when a DNA marker was 100% informative in a family. Usually, even with the type of markers used (the most informative markers in the chromosomal regions to be examined), the Lod score will not reach its maximum value.

On genetic linkage analysis, to be significant, the Lod score has to reach or exceed a value of 3 (result to 1000/1).

b—Effect of Incorrect Diagnosis on Linkage Analysis Results

The effect of attributing an incorrect diagnosis was tested on the results of the analysis (in the case of linkage to a locus) by a simulation on family Can 46 by varying the phenotype of 1, 2 or 3 individuals (Table 4).

TABLE 4
Lod score obtained for a series of distances from the marker to
the locus of the trait (maximum Lod score in bold).
1. using current diagnosis (maximum Lod score)

| | | | | | | | | |
|---|---|---|---|---|---|---|---|---|
| distance | 0.0 | 0.01 | 0.05 | 0.1 | 0.2 | 0.3 | 0.4 | a |
| Lod | 2.28 | 2.24 | 2.08 | 1.88 | 1.44 | 0.95 | 0.43 | |
| score | | | | | | | | |

2-3 individuals incorrectly diagnosed (A2, R10, R19)

| | | | | | | | | |
|---|---|---|---|---|---|---|---|---|
| distance | 0.0 | 0.01 | 0.05 | 0.1 | 0.2 | 0.3 | 0.4 | a |
| Lod | −3.89 | −1.75 | −0.90 | −0.50 | −0.14 | −0.01 | 0.01 | |
| score | | | | | | | | |

3-2 individuals incorrectly diagnosed (A2, R19)

| | | | | | | | | |
|---|---|---|---|---|---|---|---|---|
| distance | 0.0 | 0.01 | 0.05 | 0.1 | 0.2 | 0.3 | 0.4 | a |
| Lod | −2.85 | −0.75 | −0.05 | 0.22 | 0.36 | 0.30 | 0.17 | |
| score | | | | | | | | |

4-1 individual incorrectly diagnosed (R19)

| | | | | | | | | |
|---|---|---|---|---|---|---|---|---|
| distance | 0.0 | 0.01 | 0.05 | 0.1 | 0.2 | 0.3 | 0.4 | a |
| Lod | 1.24 | 1.24 | 1.23 | 1.16 | 0.94 | 0.63 | 0.27 | |
| score | | | | | | | | |

### 3. Discussion, Conclusion and Decisions

This simulation study allowed the 29 pre-selected families to be placed into 5 groups according to the maximum potential Lod score. While a linkage was significant as soon as a Lod score value of Z=3.00 was achieved, the inventors preferred to distinguish 2 groups when this criterion was verified, as the maximum simulated Lod score was very rarely reached. Thus, the probability for the real Lod score for families in group B (3≦Zmax<4) was quite low.

The families from group A were informative for genetic linkage analysis for localization of the gene/genes for premature canities. To this end, there could be no uncertainty as regards phenotype. In the case of doubt, it was recommended that the individual or even the family be excluded from the study.

However, in some of these families, the high proportion of affected/unaffected individuals (sometimes all children affected) must constitute a strong defense of the 100% genetic nature of the trait. Clearly, it cannot be excluded that in certain families the PC gene has been transmitted simultaneously by the paternal and maternal branches of the first generation. In this case, the young children were all affected (Can28, 43, 53 . . . ). In order to be able to consider these few families positively, it was highly desirable that this hypothesis be verified.

Families from group B were themselves very interesting as they allowed the sample to be expanded even if individually they could not reach a significant Lod score in the majority of cases. As a group, however, they could consolidate the Lod score, especially if it turned out that the trait was also genetically heterogeneous (slightly).

The families from group C could also be used in studies for replication of the genetic linkage results.

The families from groups D and E (Zmax<2) were not very informative for genetic linkage analysis.

Subject to a robust clinical characterization, it appears that the families of groups A, B and C were suitable for a genetic linkage analysis and they had to be included (individuals with cd-2). Genetic linkage analyses carried out on insufficiently characterized samples were destined for failure or to produce a “soft” result (inaccurate locus). Given that in such analyses, certain parameters can not be totally under control (in particular the informativity of the genotypes) and that genetic heterogeneity, which is still possible, renders the job more difficult (as it reduces the power of the analysis over the set of families), it thus appears vital to hold all the possible advantages right from the start. In this first step, the inventors thus strongly recommended that the phenotype for each individual with an appropriate availability code (cd=2) be carefully verified before inclusion (phenotype confirmed, or exclusion from sample/family).

### B—Period 2: Capture of Samples, Confirmation of Phenotypes and New Estimates of Study Potential

On the basis of the results from period 1, the 19 families (groups A, B and C) retained to form a base in which the pedigrees will be sampled for genetic linkage analysis were contacted to confirm the PC diagnosis and capture a series of affected and unaffected individuals.

This medical phenotype verification campaign allowed a large number of PC diagnoses to be confirmed, but not all as planned. The refusal of a few key individuals (with PC) to participate in the project, the death of some others and a readjustment of part of the phenotypes meant that a certain number of families could not be retained and that the informativity potential of some other families was reduced.

1—Re-Estimation of Study Potential after Phenotype Confirmation

To re-estimate the potential of the study after phenotype verification, the inventors simulated a linkage analysis for the 8 families who could still be informative. Table 5 shows the results obtained for this set of 8 families in 3 situations of genetic heterogeneity (0%, i.e. all families linked to the same locus, 50% or only half the families linked to the same locus, 70% or only about a third of the families being linked).

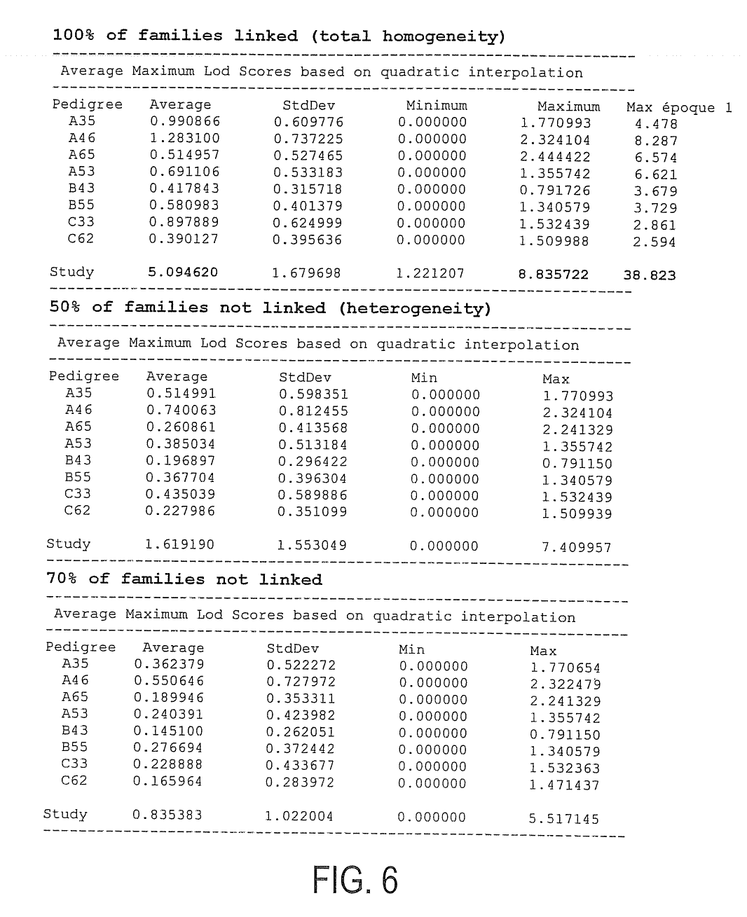

TABLE 5
Potential Lod scores as a function of the degree
of genetic heterogeneity of PC
The results, detailed by family, are shown in the table in FIG. 6.

| degree of | | | | | max |
|---|---|---|---|---|---|
| heterogeneity | mean | SD | minimum | maximum | period 1 |
| 0% | 5.094620 | 1.679698 | 1.221207 | 8.835722 | 38.823 |
| 50% | 1.619190 | 1.553049 | 0.000000 | 7.409957 | — |
| 70% | 0.835383 | 1.022004 | 0.000000 | 5.517145 | — |

### 2—Conclusion

Continuing in our efforts to optimize the sample for the linkage analyses; 4 supplementary families were identified (Table 6; 2 families—can65B and can46B—were collateral branches of families can65 and can46) and the phenotypes were verified again.

After a new simulation (Table 7), it was shown that the general maximum Lod score for linkage was very slightly higher (Z=8.91) if the 12 retained families (strictly defined phenotypes) were linked to the same locus. The expected increase in the Lod score by adding the new families was, however, reduced by the correction and hardening of the phenotypes.

TABLE 6
Final list of families

| | |
|---|---|
| 1 | c103974 |
| 2 | F104512 |
| 3 | CAN33 |
| 4 | CAN35 |
| 5 | CAN43 |
| 6 | CAN46 |
| 7 | CAN46B |
| 8 | Can53 |
| 9 | CAN55 |
| 10 | CAN62 |
| 11 | CAN65 |
| 12 | CAN65B |

TABLE 7
New simulation after including new families.

| degree of | | | | |
|---|---|---|---|---|
| heterogeneity | mean | SD | minimum | maximum |
| 0% | 4.752321 | 1.748924 | 0.356854 | 8.914062 |
| 50% | 1.535356 | 1.418022 | 0.000000 | 6.078132 |
| 70% | 0.719737 | 0.978920 | 0.000000 | 5.282466 |

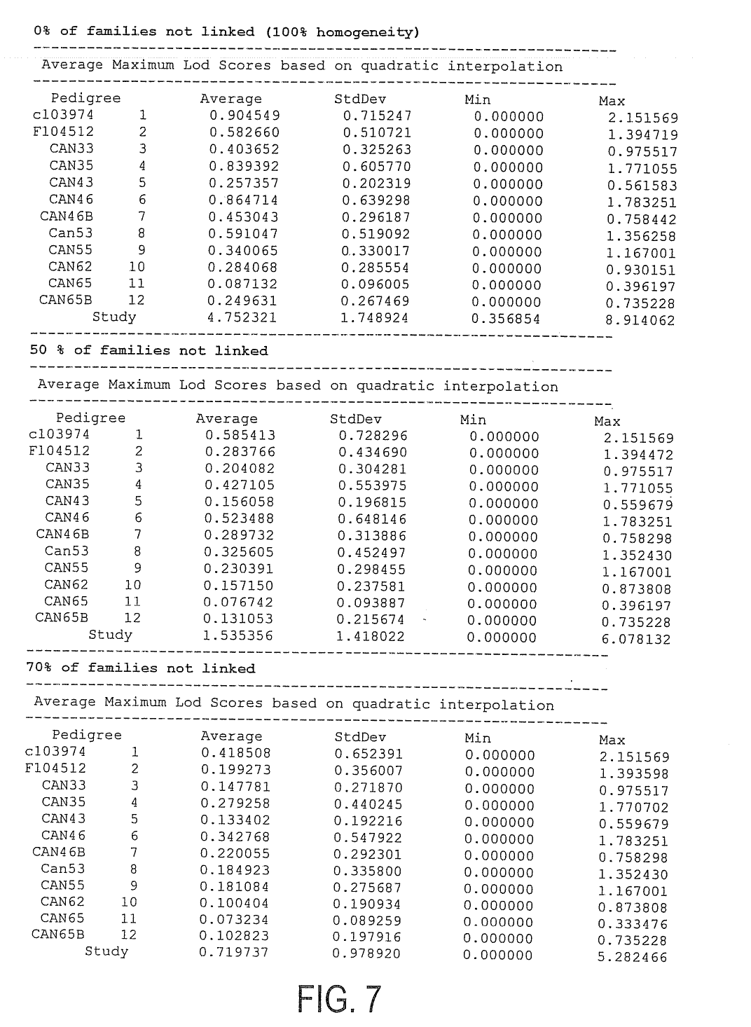

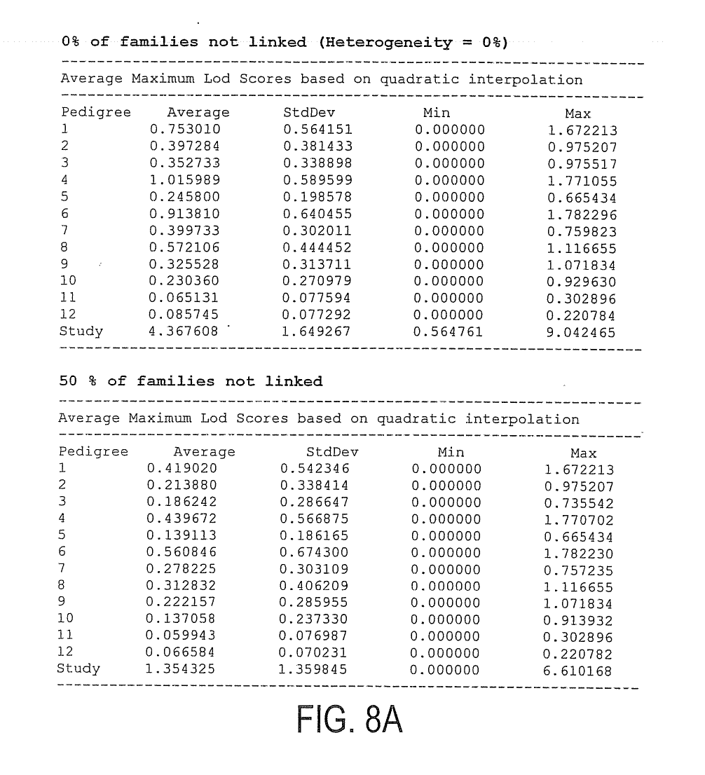

The potential Lod scores are expressed as a function of the degree of genetic heterogeneity. The detailed results are shown in the table in FIG. 7.

The power of the sample could also be observed from the viewpoint of the number of replications which reached or exceeded the Lod scores Z=1.0, Z=2.0, Z=3.0 respectively and which gave an approximation of the chance of finding a significant link (Table 8).

TABLE 8
Probabilities (%) of reaching or exceeding a Lod score
of 1, 2 or 3 for each degree of heterogeneity

| | degree of | | | |
|---|---|---|---|---|
| | heterogeneity | 0% | 50% | 70% |
| Lod score | 1.000 | 99.500 | 57.000 | 27.000 |
| | 2.000 | 95.500 | 31.000 | 8.500 |
| | 3.000 | 84.500 | 14.000 | 3.500 |

C—Period 3: Genetic Linkage Analysis of PC with Candidate Regions

### 1—Hypothesis

The region 6p21 was termed the “candidate region” (CR) as it is associated with premature canities via auto-immune diseases (Biermer's disease, Graves' disease, thyroiditis, myasthenia) with which the trait is associated.

The region 3p14.1-p12.3 was also termed a “candidate region” (CR) as it is associated with premature canities in (Klein) Waardenburg's syndrome (Type IIA) disease with which the trait is associated.

### 2—Final Composition of Families

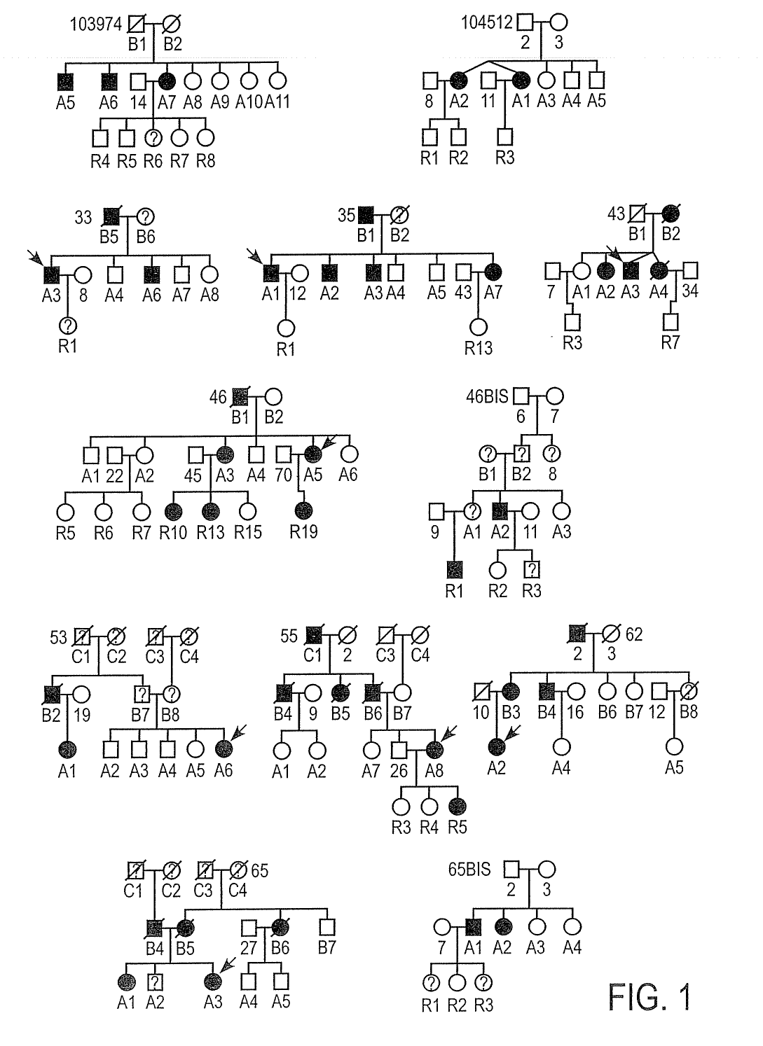

With the aim of optimizing the technique, a sample of 92 affected and unaffected individuals out of the 12 families was retained (see FIG. 1). This selection was made as a function of the potential informativity of each individual and confirmed by a new linkage simulation. Adding a few individuals led to a slight increase in the potential Lod score (from 8.91 to 9.04, using complete homogeneity, Table 9) and thus in the power of the sample (Table 10).

TABLE 9
New simulation on final families to investigate
candidate chromosomal regions.

| degree of | | | | |
|---|---|---|---|---|
| heterogeneity | mean | SD | minimum | maximum |
| 0% | 4.367608 | 1.649267 | 0.564761 | 9.042465 |
| 50% | 1.354325 | 1.359845 | 0.000000 | 6.610168 |
| 70% | 0.666549 | 0.891330 | 0.000000 | 4.988076 |
| 90% | 0.222622 | 0.378039 | 0.000000 | 2.528617 |

The potential Lod scores are expressed as a function of the degree of genetic heterogeneity. The detailed results are shown in the table in FIG. 8.

TABLE 10
Probabilities (%) of reaching or exceeding a Lod score of
1, 2 or 3 for each degree of heterogeneity
The results detailed are shown in the table in FIG. 9.

| | degree of | | | | |
|---|---|---|---|---|---|
| | heterogeneity | 0% | 50% | 70% | 90% |
| Lod score | 1.000 | 98.000 | 48.500 | 21.500 | 4.000 |
| | 2.000 | 94.500 | 23.500 | 9.500 | 0.500 |
| | 3.000 | 79.000 | 12.000 | 3.000 | 0.000 |

### 3—Microsatellite Markers Used

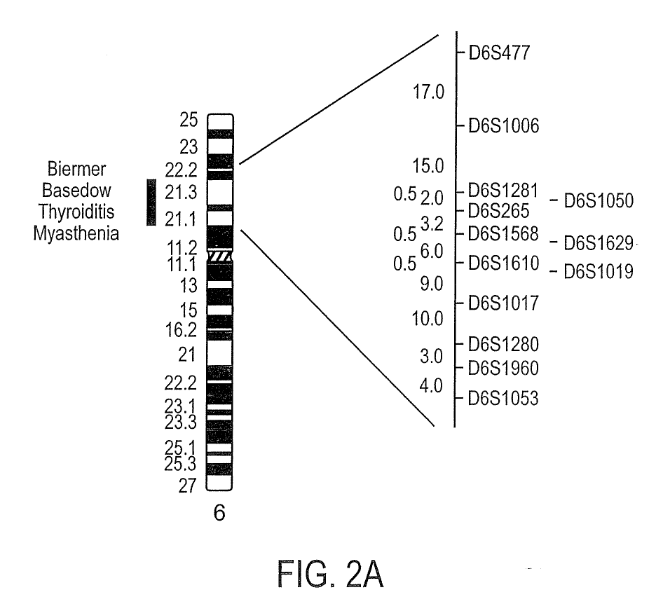

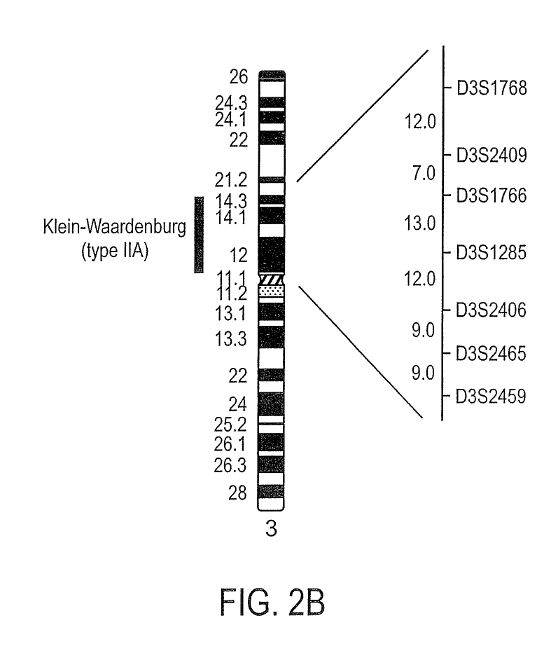

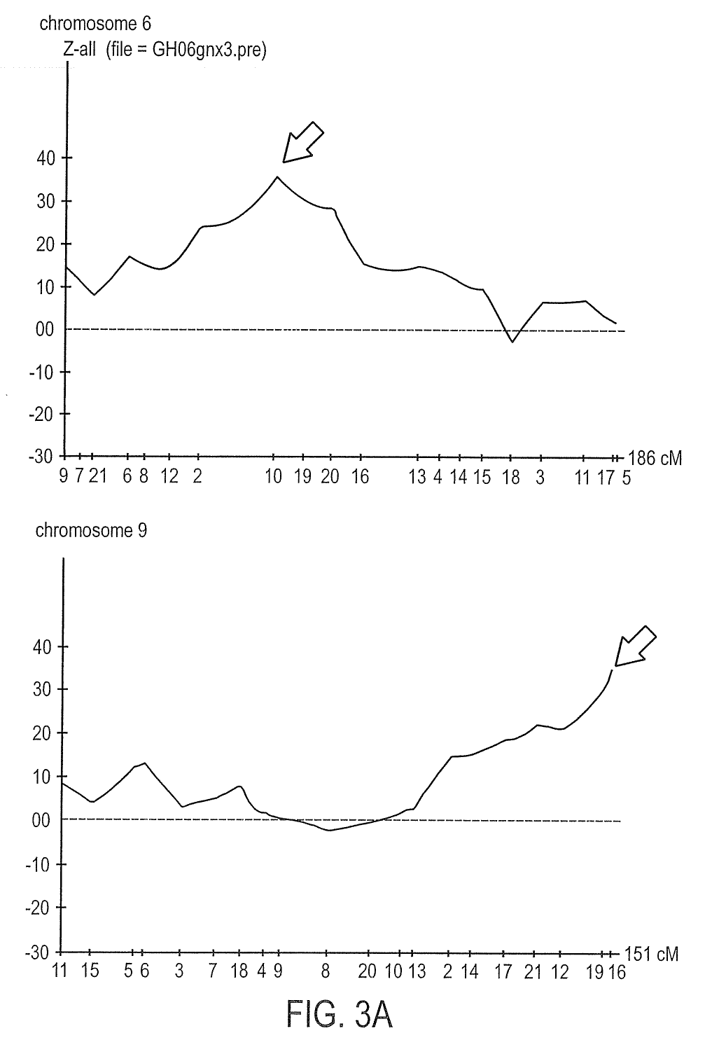

The distribution of markers over the candidate region of chromosome 6 is shown in FIG. 2A.

The distribution of markers over the candidate region on chromosome 3 is shown in FIG. 2B.

### 4—Linkage Analyses

Several types of linkage analysis were carried out to increase the probability of observing an existing linkage between the chromosomal regions and PC.

For this analysis, the following two approaches were made:
2-point (iterative analysis between the trait and the markers taken one at a time);
multipoint (analysis for each chromosome using a marker map placed as a function of their respective distances).
   1/ Analyses with defined parameters, parametric (PL): transmission mode (dominant), trait frequency (1%), equifrequency of alleles of test markers and penetrance (90% mutant heterozygotes 100% mutant homozygotes). 2-point and multipoint method.
   2/ Independent analysis of trait transmission mode, non-parametric (NPL): Deviation analysis of the proportion of shared alleles for each pair of affected individuals (in each family using identity by descent) compared with random transmission. In the multipoint analysis, all affected pairs (all-pairs, score Z-all=log₁₀ of p-values, and p-values) were considered. In the 2-point method, sibling pairs were studied (affected sib-pair, p—values).
   5—Results The results are shown in Table 11.

TABLE 11
Results of linkage analysis on candidate regions.
When only one position is indicated, and not the name of a marker, this means
that the position is intermediate between two markers.

| | | global | | | | | |
|---|---|---|---|---|---|---|---|
| | | 2-point | | multipoint | | | |
| | | | marker/ | | marker/ | | marker/ |
| chromosome | region | Lod | position, cM | lod | position, cM | NPL | position, cM |
| 3 | 3p14.1-p12.3 | 1.03 | D3S1285/90 | 1.55 | 82 | 2.58 | D3S2409/70 |
| | | @ 0.2 | | | | | |
| 6 | 6p21 | 1.12 | D6S1017/54 | 1.28 | 58 | 3.56 | D6S1017/54 |
| | | @ 0.1 | | | | | |

| | | positive families | | | | | | |
|---|---|---|---|---|---|---|---|---|
| | | | | | multipoint | | | |
| | | 2-point | | | | position, | | position, |
| chromosome | region | ID | lod | marker | lod | cM/marker | NPL | cM/marker |
| 3 | 3p14.1-p12.3 | 53 | 1.54 | D3S1285 | 1.61 | D3S1285 | 1.58 | D3S1285 |
| 6 | 6p21 | 35 | 1.67 | D6S1629 | 0.93 | 40/D6S1629 | | |

### 6—Discussion and Conclusion

This study highlighted a predominant locus for predisposition to premature canities on chromosome 6p21-p12 in the region of the HLA genes (around D6S1017; maximum NPL scores=3.52, p=0.000514, HLod:=2.01). The linkage region (with a degree of confidence of 99%, NPL>2.50) is located between positions 41 and 67 on the map of selected markers. Non-parametric analysis supplies a series of significant values between positions 47 cM and 58 cM (between D6S1019 and D6S1280). The Lod score, although suggestive, also supports localization of an important gene on chromosome 6p.

These results positively document the association assumptions relating to the chromosomal region 6p21 as regards premature canities.

It should be noted that one family (CAN35) showed a relatively high linkage.

The linkage result on chromosome 6p21 in the HLA region is very encouraging if it is remembered that the literature supplies few examples of non-parametric analyses that are this informative. It provides a promising starting point for identifying genes for susceptibility to premature canities using strategies associated with polymorphisms.

This study also highlights a locus strongly suggesting a predisposition to premature canities on chromosome 3p14-p12 (towards markers D3S2409 and D3S1766; multipoints NPL=2.58 at position 12 and HLod:=1.55 at position 24).

This result positively documents the association assumptions relating to the chromosomal region 3p14-p12 as regards premature canities.

It should be noted that one family (CAN35) showed a relatively high linkage for this chromosomal region.

D—Period 4: Global Genetic Linkage Analysis of PC with the Genome

Global genome analysis allowed all of the chromosomes to be visited (global probe) to find regions which are involved and possibly a major locus which would govern premature canities. This major analysis also allowed the degree of genetic heterogeneity of PC to be estimated.

### 1—Content of Families

These were the same families as those studied in the candidate region phase (period 3), however with certain adjustments as regards their content (see Table 12). Some less informative members were replaced by others who had been recruited more recently, or for whom a diagnosis had been made later on.

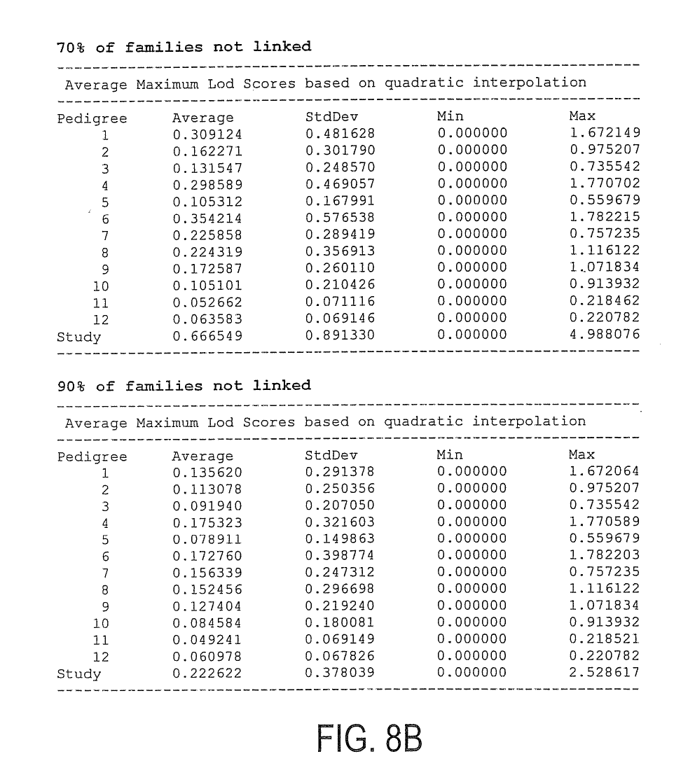

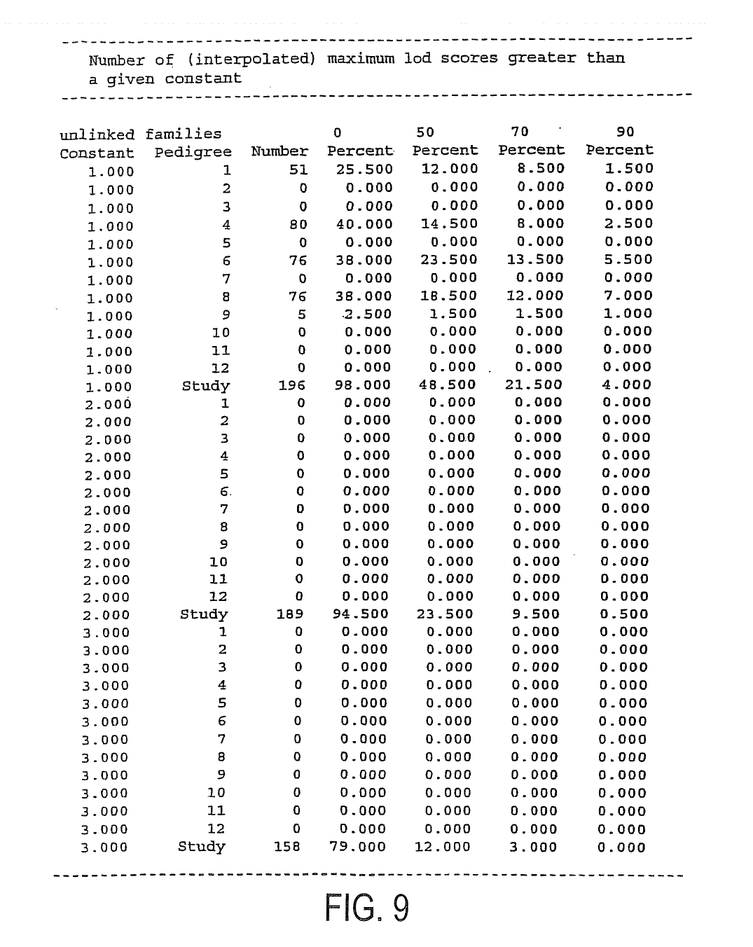

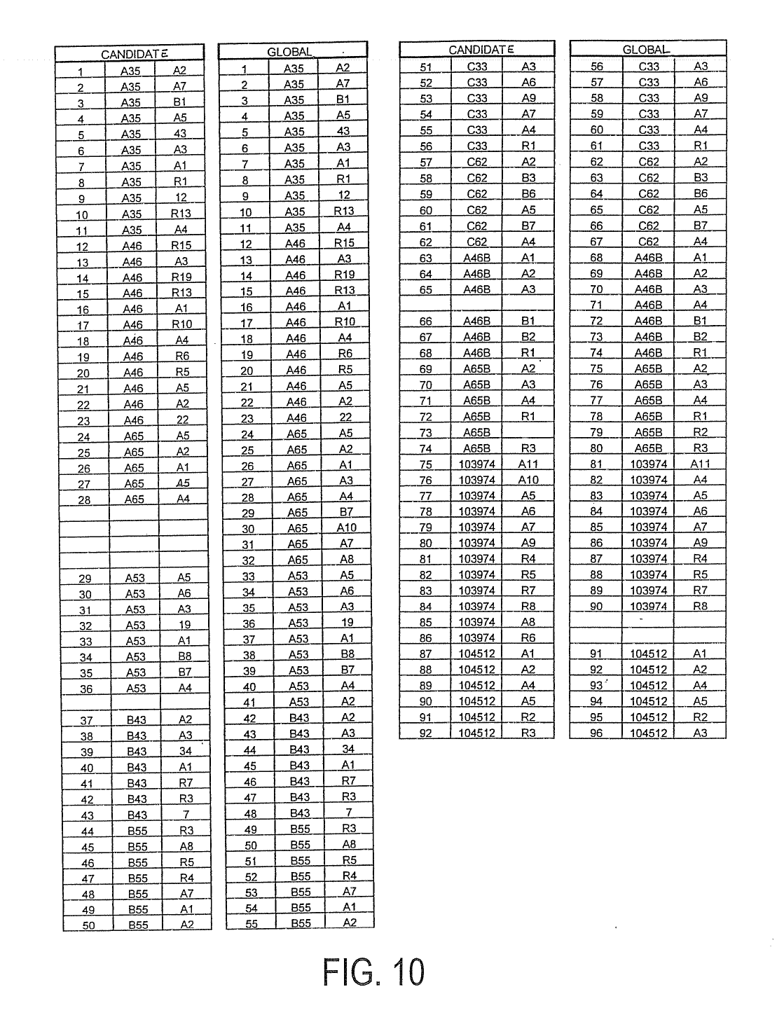

TABLE 12
Comparison of the number of individuals studied in each family
between the region-candidate analysis and the genome-global analysis
(detailed composition of families, table in FIG. 10)

| families | candidate region | global genome |
|---|---|---|
| A35 | 11 | 11 |
| A46 | 12 | 12 |
| A65 | 5 | 9 |
| A53 | 8 | 9 |
| B43 | 7 | 7 |
| B55 | 7 | 7 |
| C33 | 6 | 6 |
| C62 | 6 | 6 |
| A46B | 6 | 7 |
| A65B | 6 | 6 |
| 103974 | 12 | 10 |
| 104512 | 6 | 6 |
| total individuals | 92 | 96 |

In order to confirm the benefit of the change in the sample, a new linkage analysis simulation was carried out for the different genetic heterogeneity situations (Table 13 and the tables in FIGS. 11A, 11B, 11C and 11D). The Lod scores showed a favorable change. Considering the possible mean Lod score, it was possible to obtain a significant result (Z>3.0) for a heterogeneity reaching 20% (i.e. ⅕ of families not linked to locus). In fact, these results were highly conservative and it was possible to reach significance with a far more heterogeneous sample (i.e. 50-70% with the use of microsatellite markers showing a mean heterozygosity of 0.7).

TABLE 13
Potential Lod scores as a function of the
degree of genetic heterogeneity of PC.

| degree of | | | | |
|---|---|---|---|---|
| heterogeneity | mean | SD | minimum | maximum |
| 0% | 4.751841 | 1.749689 | 0.334792 | 9.338889 |
| 20% | 3.115806 | 1.866821 | 0.024841 | 8.780452 |
| 50% | 1.378378 | 1.467071 | 0.000000 | 7.508479 |
| 70% | 0.672997 | 0.904472 | 0.000000 | 5.226281 |
| 90% | 0.242418 | 0.402384 | 0.000000 | 2.722478 |

The results detailed here are shown in the tables in FIG. 11.

The power of the sample can also be observed from the viewpoint of the number of replications (genotypes) which achieve or exceed the Lod scores Z=1.0, Z=2.0, Z=3.0 respectively. The probability of finding a significant linkage (Table 14 and FIG. 12) with ⅘ of the families linked to the same locus was 50%. This result is based on a mean Lod score which is conservative.

TABLE 14
Probabilities (%) of reaching or exceeding a Lod score of
1, 2 or 3 for each degree of heterogeneity
The results detailed are shown in the table in FIG. 12.

| degree of | | | | | |
|---|---|---|---|---|---|
| heterogeneity | 0% | 20% | 50% | 70% | 90% |
| 1.000 | 99.000 | 87.500 | 47.000 | 23.000 | 5.500 |
| 2.000 | 93.500 | 69.500 | 25.500 | 11.500 | 0.500 |
| 3.000 | 84.00 | 48.500 | 15.000 | 3.500 | 0.000 |

Thus, the analyses could indicate significant linkages if the heterogeneity of the sample did not exceed 20% (i.e. only ⅕ of families not linking to a single major locus), but it is still possible to identify a linkage in the case in which half of the families are not linked to this locus.

### 2—DNA Markers

DNA from 96 individuals belonging to the selected 12 families was genotyped for 400 polymorphous markers distributed over 22 autosomes and the X chromosome (Table 15) using a mean inter-marker interval of 9.2 cM (density). These are DNA microsatellites which are composed of dinucleotide (CA)ₙ type tandem repeats from the Généthon collection (Evry, France).

TABLE 15
Number of markers analyzed for each
chromosome genome-wide scan

| chromosome | number of markers |
|---|---|
| 1 | 31 |
| 2 | 30 |
| 3 | 23 |
| 4 | 22 |
| 5 | 22 |
| 6 | 20 |
| 7 | 22 |
| 8 | 14 |
| 9 | 20 |
| 10 | 20 |
| 11 | 18 |
| 12 | 19 |
| 13 | 14 |
| 14 | 14 |
| 15 | 14 |
| 16 | 13 |
| 17 | 15 |
| 18 | 14 |
| 19 | 12 |
| 20 | 13 |
| 21 | 5 |
| 22 | 7 |
| X | 18 |
| total | 400 |

The observed mean degree of heterozygosity was 0.70, and the mean size of the inter-marker interval was 9.2 cM.

### 3—Linkage Analyses

For this global approach, the inventors carried out several types of analyses to optimize their performance in finding a linkage between a region of the genome and PC. Parametric analysis, which was more powerful, takes into account the mode of transmission of the trait and is the most suitable in the case of monogenic traits. Non-parametric analysis can identify a linkage even if the assumed mode of transmission is erroneous, and is also more robust in the case of multigenic traits.

For each of these analyses, the inventors used the methods mentioned above, the 2-point method (iterative analysis between the trait and each marker) and the multipoint method (global analysis on each chromosome using a marker map).

a—Analyses with Defined Parameters, Parametric (PL)
transmission mode (dominant),
trait frequency (1%),
allelic equifrequency of alleles of all markers,
defined penetrances (90% mutant heterozygotes 100% mutant homozygotes),
2-point and multipoint methods,
linkage probability scores expressed as:
   Lod score Z (homogeneous sample);
   Lod score ZH (heterogeneous sample) and degree of heterogeneity α (proportion of families which are not linked to this locus).
      b—Independent analysis of transmission mode of trait, non-parametric (NPL)

This is a deviation analysis of the proportion of alleles shared by pairs of affected individuals compared with random transmission of alleles (identity by descent). The inventors considered all pairs of affected individuals for the multipoint analysis, and pairs of siblings for the 2-point analysis.

The linkage probability scores were expressed as:
NPL or Z-all (log10 of p value) for the multipoint method over all pairs of affected individuals;
“p” value for the 2-point method over affected sib pairs.

### 4—Results

a—Detailed Results

For chromosome 6 in particular, the 20 markers belonging to the global genome collection (GG) were first analyzed, then the analysis was repeated, adding the 13 markers used for the candidate region (CR) analysis to the 20 global genome markers.

FIG. 3 shows the NPL scores obtained for the non-parametric linkage analysis on chromosomes 6, 9, 3, 5 and 11.

Table 16 records the best results for each type of analysis (PL, NPL) discussed below:

TABLE 16
Best results for each type of analysis (PL, NPL) on markers used for
GG analysis except for * (* 33 markers, CR + GG).

| | | | |
|---|---|---|---|
| | | | 2-pt (2P) or |
| chromosome | position/pter | score | multipoint (MP) |

non-parametric

| | |
|---|---|
| npl > 3.0 | Z-all |

| | | | |
|---|---|---|---|
| 6 | 57 | 3.59 | MP* |
| 9 | 151 | 3.37 | MP |

| | |
|---|---|
| npl > 2.5 | Z-all |

| | | | |
|---|---|---|---|
| 3 | 72 | 2.62 | MP |
| 11 | 106 | 2.61 | MP |

| | |
|---|---|
| npl > 2.0 | Z-all |

| | | | |
|---|---|---|---|
| 9 | 131.00 | 2.13 | MP |
| 3 | 72 | 2.12 | MP |

| | |
|---|---|
| p < 1 × 10−4 | p |

| | | | |
|---|---|---|---|
| 6 | 154.1 | 0.000012 | 2P |

parametric

| | |
|---|---|
| Lod > 2.0 | Lod |

| | | | |
|---|---|---|---|
| 5 | 164.2 | 2.007 | 2P |

| | |
|---|---|
| Lod > 1.5 | Lod |

| | | | |
|---|---|---|---|
| 11 | 106 | 1.5288 | MP |

| | |
|---|---|
| Lod > 1.0 | Lod |

| | | | |
|---|---|---|---|
| 5 | 168 | 1.1118 | MP |
| 6 | 61 | 1.4294 | MP* |
| 6 | 89.7 | 1.458 | 2P |

### i) At end of PL:

a—A Lod score (2P) ZH=2.007 was obtained on the long arm on chromosome 5 (position 164.2 cM from the upper telomere), i.e. in a position ¾ on the 5q31-q32 band.
b—Intermediate Lod scores (1.5<ZH<2.0)
chromosome 11q14-q22, position 106 (MP-ZH=1.53)
   c—Interesting Lod scores (1.0<ZH<1.5)
chromosome 5q31-q32, position 168 (MP-ZH=1.11)
chromosome 6p21-p12, position 61 (MP-ZH=1.43)
chromosome 6q13-q14, position 90 (2P-ZH=1.46)

### ii) At End of NPL

We distinguished log₁₀ scores for the allelic sharing deviation study, identity by descent (IBD), for all affected pairs (multipoint Z-all study scores) as well as p values for pairs of affected siblings (affected sib-pairs, 2-point study scores, p-values).

The best scores are shown as their rank compared with the limit of significance:
Z-all>3, 2.5<Z-all<3, 2.0<Z-all<2.5
p<10⁻⁵ and 10⁻⁵≦p≦10⁻⁴
   a—Z-All>3.0 Scores

On chromosome 6p21-p12, the score reached using the global genome markers had a Z-all=3.52 in position 71 (between markers D6S1610 and D6S257). However, the accuracy of the locus was probably affected by the large distance between these 2 markers of the global genome collection which was much larger than the observed mean interval (26.11 cM).

Because of the size of this interval, a complementary analysis was carried out on the set of 33 markers used during the GG and CR analyses. This analysis produced a higher score (Z-all=3.59) for the 57 cM position (see Table 16).

The second best score was achieved for chromosome 9q31-q32, position 151 (Z-all=3.37).

b—2.5<Z-All<3.0 Scores
chromosome 3p14-p13, position 72 (Z-all=2.62)
chromosome 11q21-q22, position 106 (Z-all=2.61)
   c—2.0<Z-All<2.5 Scores
chromosome 9q31-q32, position 131 (Z-all=2.13)
chromosome 3q21, position 101 (Z-all=2.15)
   and p-values<10⁻⁵ and 10⁻⁴.
   d—p<1×10⁻⁴
chromosome 6q31.3-q33, position 154 (p=0.000012)

iii) Loci simultaneously identified by PL and NPL:

| | PL | NPL |
|---|---|---|
| 6p21-p12, position 57-61 | 1.42 | 3.59 |
| 11q14-q22, position 106 | 1.52 | 2.6 |

### 5—Discussion and Conclusion

Two scores which were significant or on the border of significance depending on whether the trait was considered to be monogenic or multifactorial (Lander and Kruglyak, 1995) were observed for chromosomes 6p21-p12 (NPL MP Z-all=3.59) and 9q31-q32 (NPL MP Z-all=3.37).

For these 2 loci, the most robust was that of 6p21-p12 which was reinforced by a MP-PL Lod score, which although average, maximized at ZH=1.42).

A further locus also appeared to be fairly interesting, and was located on the chromosome 11q14-q22 as the PL and NPL scores maximized at the same position 106 (Z-all 2.61, PL 1.52). The NPL score was in the suggestivity range in a case of monogenism or in a case of multigenism (suggestivity: monogenism 2<Z-all<3; multigenism 2.2<Z-all<3.6).

Finally, the locus 5q31-q32 with the best PL Lod score (2P) (ZH=2.00) was also located within suggestivity values (in monogenism).

A p value series had to be added for the sib affected analyses which were also within the suggestivity range (chromosomes 6q31.3-q33). These loci are also to be considered.

### D—Discussion and General Conclusions

At the end of the various analysis periods, several chromosomal regions had been identified or suggested.

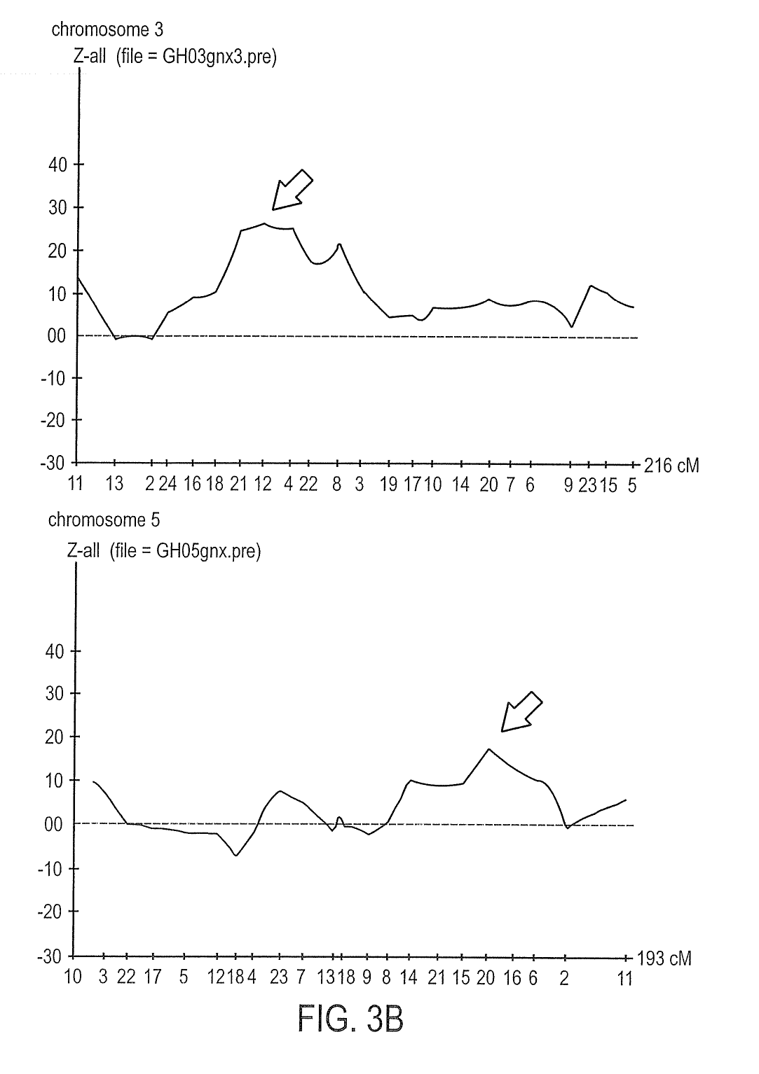

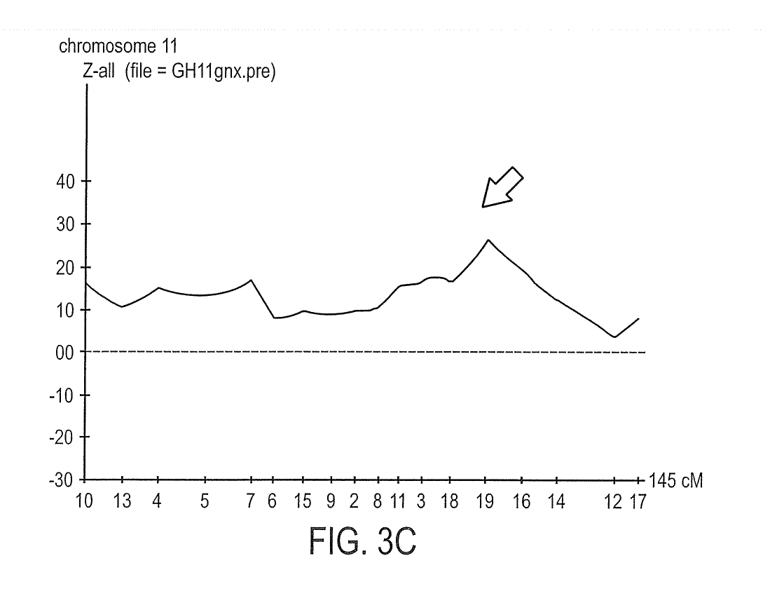

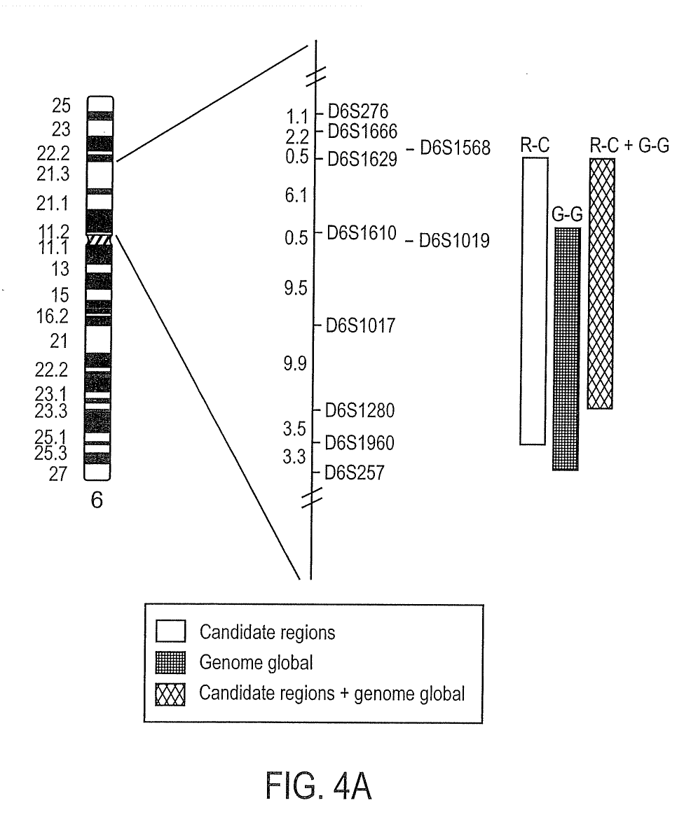

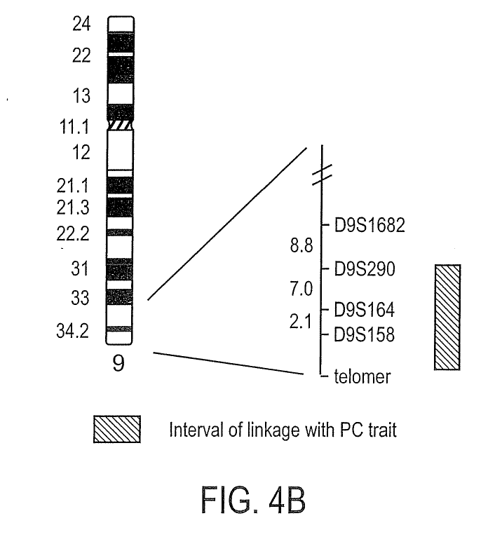

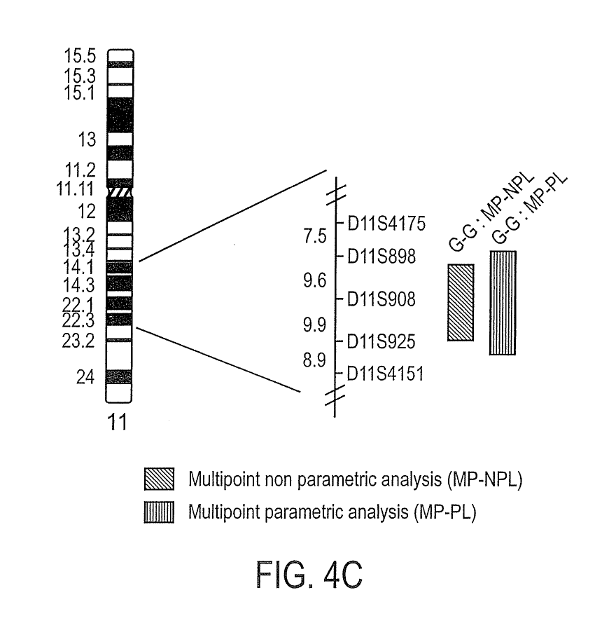

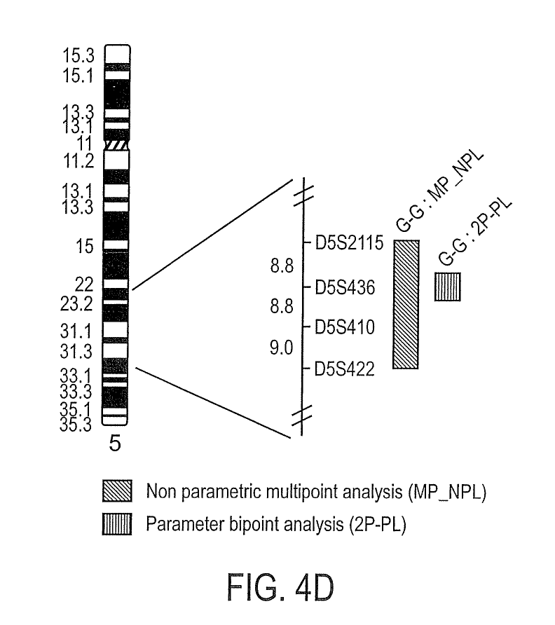

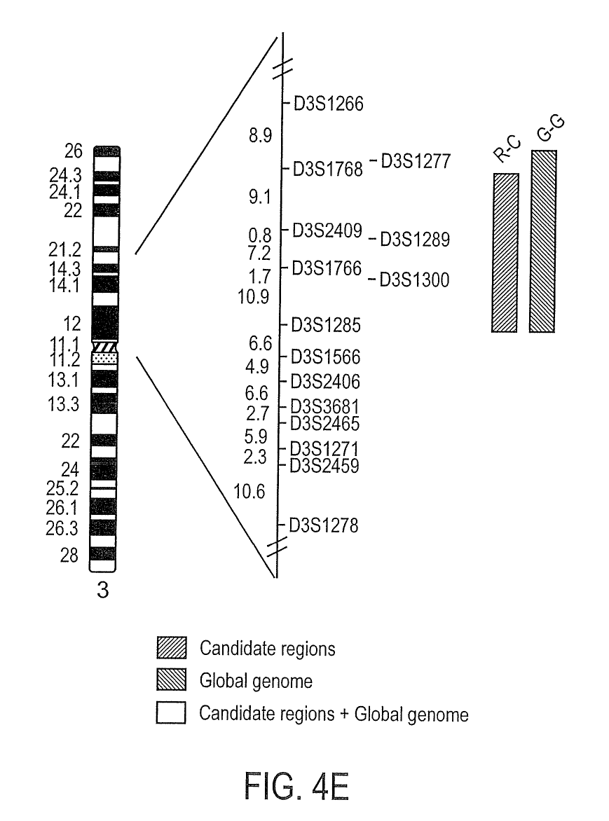

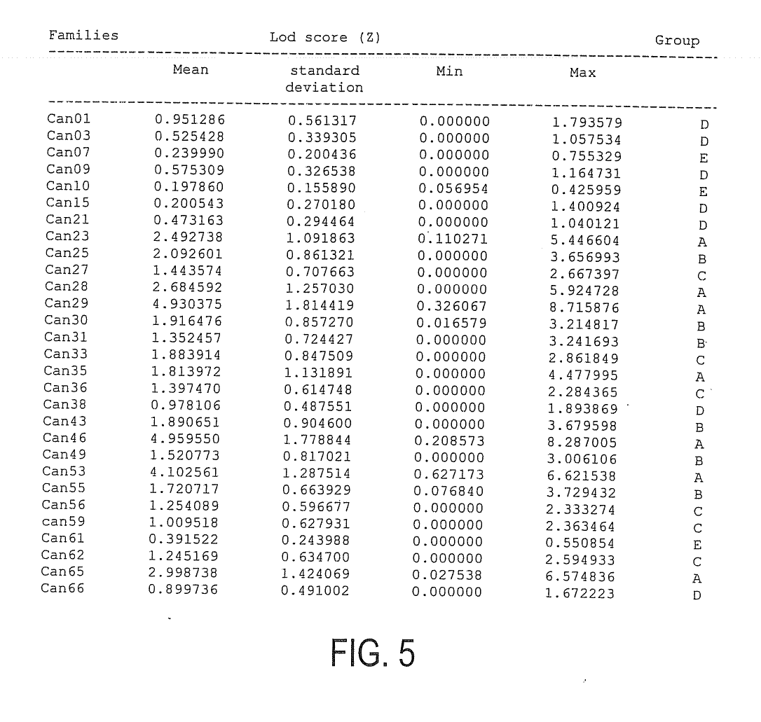

The region 6p21-p12 (see FIG. 4A) recorded the best consensus for a genetic linkage to the PC trait. The discord between the parametric and non-parametric analyses generated some uncertainty as regards the importance of the role of the gene or genes in the pathophysiology of canities.

The second locus is on chromosome 9q31-q32 with an NPL score which is almost significant (Z-all=3.37).

a—Chromosome 6p21-p12

The limits of the region designated by the linkage analysis are located at position 41 (upper limit, Z-all=2.02) and position 95 (lower limit, Z-all=2.09), i.e. 54 cM. It is possible to reduce this region to a length of 15 cM by considering the 65 cM and 80 cM limits (between D6S1610 and D6S257 respectively; 20 marker analysis) or 50 cM and 73 cM (between D6S1629 and D6S1280 respectively; 33 marker analysis).

The region identified contains the genes of the major histocompatibility complex (MHC, HLA). However, it cannot be excluded that a gene independent of HLA could be involved in governing or a susceptibility to the PC trait.

b—Chromosome 9q34

The inventors place the proximal limit of this region starting from the position which has a Z-all score of 2.5 on the D9S290 marker; the distal limit is placed on the telomere of the long arm on chromosome 9 (towards the D9S158 marker). This region extends over a length of 10 cM (FIG. 4B).

c—Chromosome 11q14-q22

The inventors identified a region between positions 100 and 115 (between D11S898 and D11S925) (FIG. 4C).

d—Chromosome 5q31-p32

This region had the same PL and two-point result for these analyses which was located at a recombination fraction (theta) 0.14 (about 14 cM) from the D5S422 marker. By placing itself at this distance from D5S422 towards the top of the map (position 149), we arrive in the vicinity of the locus which has the best NPL multipoint score for chromosome 5 (Z-all=1.70, towards marker D5S436). A certain consensus also appeared for this locus (FIG. 4D).

e—Chromosome 3p14.1-p12.3

This is a region of almost 30 cM between positions 60 and 87 (between D3s1277 and D3s1285 (FIG. 4E).

### Example 2

### Analysis of Regions of Interest Using SNPs (Single Nucleotide Polymorphism)

Subsequent to the work presented in Example 1, the inventors continued the analysis of the regions of chromosomes 6 and 9 using techniques based on SNPs, to highlight the genes involved in premature canities.

SNPs (single nucleotide polymorphisms) represent a form of polymorphism which is particularly widespread through the human genome and is very stable. The number of SNPs is estimated to be about 0.8 SNPs per 1000 nucleotides (coding and non coding sequences together) which allows a true map of the human genome to be established using SNPs. SNPs are often classified into different categories, in particular depending on whether or not they are in a coding region, in a regulating region or in another non coding region of the genome, whether the polymorphism modifies the coded amino acid or not, etc.

Following the “Human Genome Project”, SNPs are better known and recorded, as well as their position in the genome (GDB).

Different methods have been developed to highlight these polymorphisms between different individuals, often based on methods used to detect point mutations (RFLP-PCR, hybridization with specific allele oligomers, mini-sequencing, direct sequencing, etc).

In the context of the present application, the inventors have used MALDI-TOF techniques (matrix-assisted laser desorption/ionization time-of-flight mass spectrometry) to detect the different alleles of candidate SNPs. The skilled person will have more details of this technique, and they have been described in many publications (Stoerker J et al, Nat Biotechnol 2000, November; 18(11): 1213-6 and Tang K et al, Proc Natl Acad Sci USA 1999 August, 96, 10016-20).

In a first stage, the inventors defined the regions of chromosomes 6 and 9 to be analyzed using SNPs very accurately. In a second step, 1500 SNPs belonging to the above regions were pre-selected on the basis of certain criteria (candidate SNPs in silico) and 697 were retained following an experimental validation step. In a subsequent step, the inventors assembled DNA from different individuals with premature canities and “control” individuals into different groups, then genotyped these different groups using 450 SNPs selected out of the 697. At the end of this genotyping, the results allowed 76 SNPs to be defined for subsequent individual genotyping (no longer on groups).

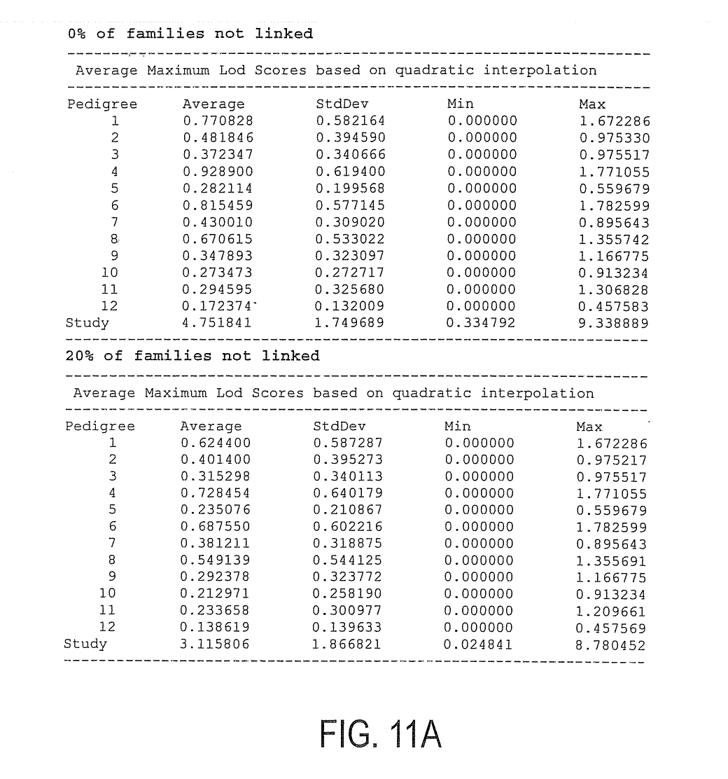

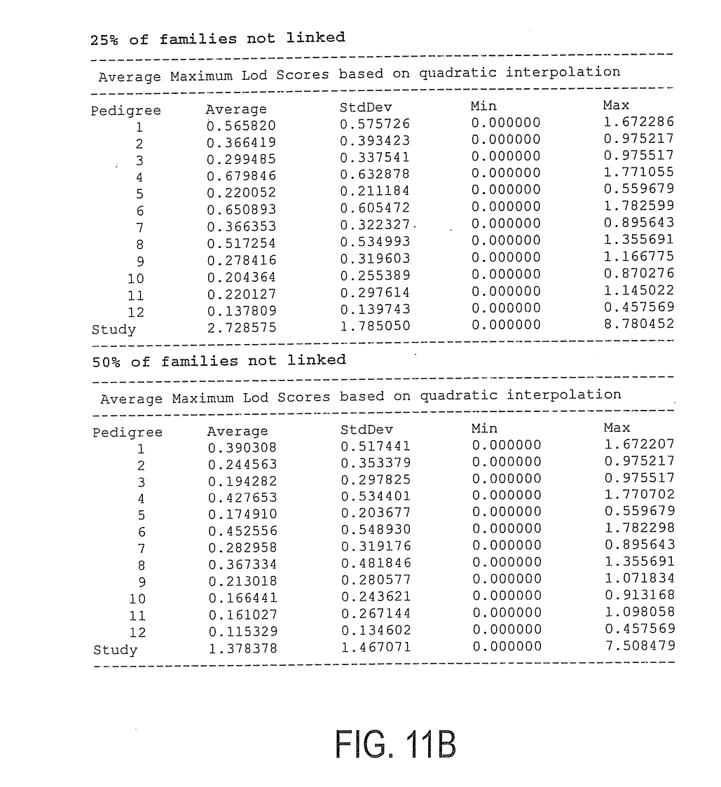

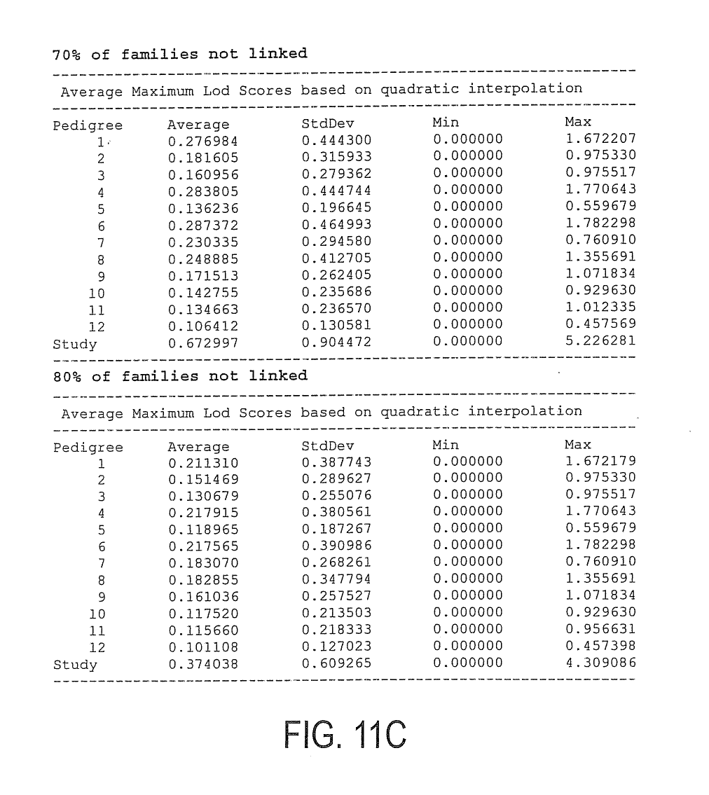

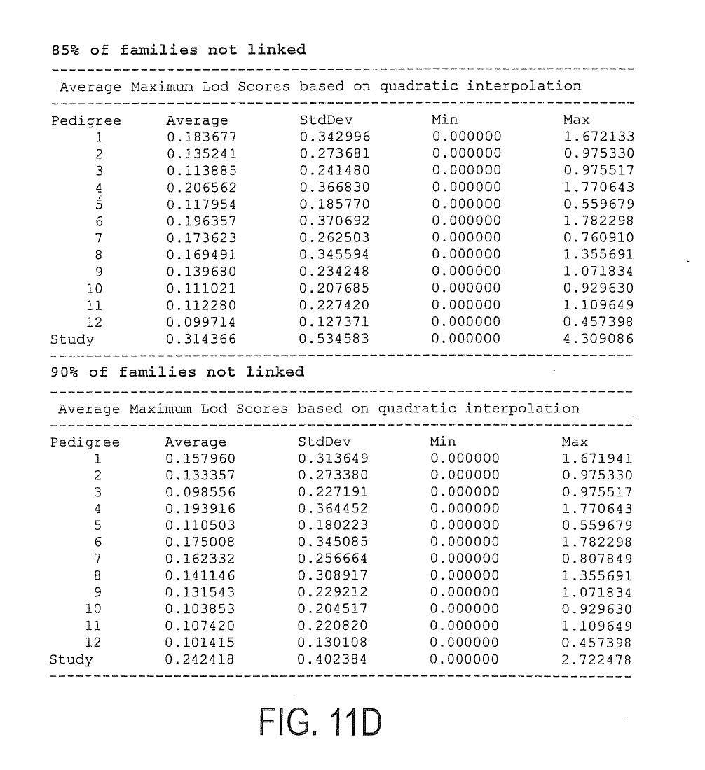

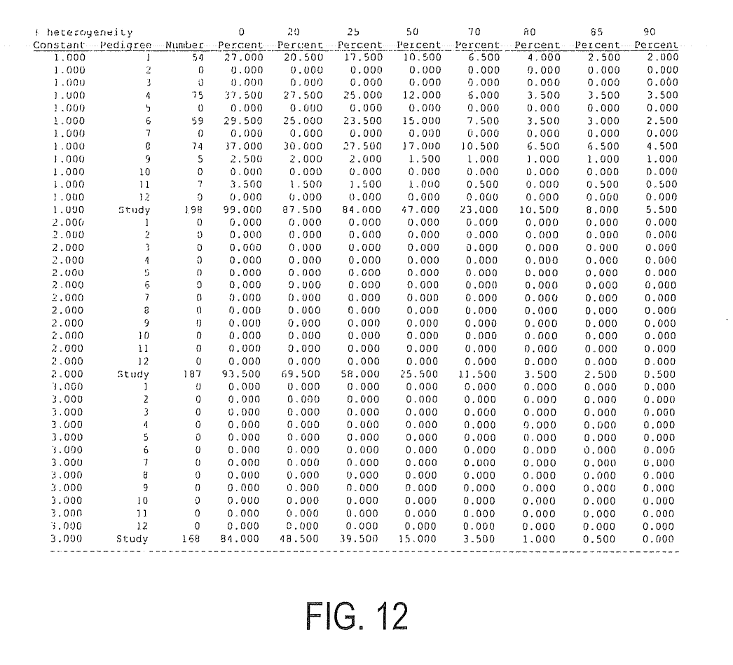

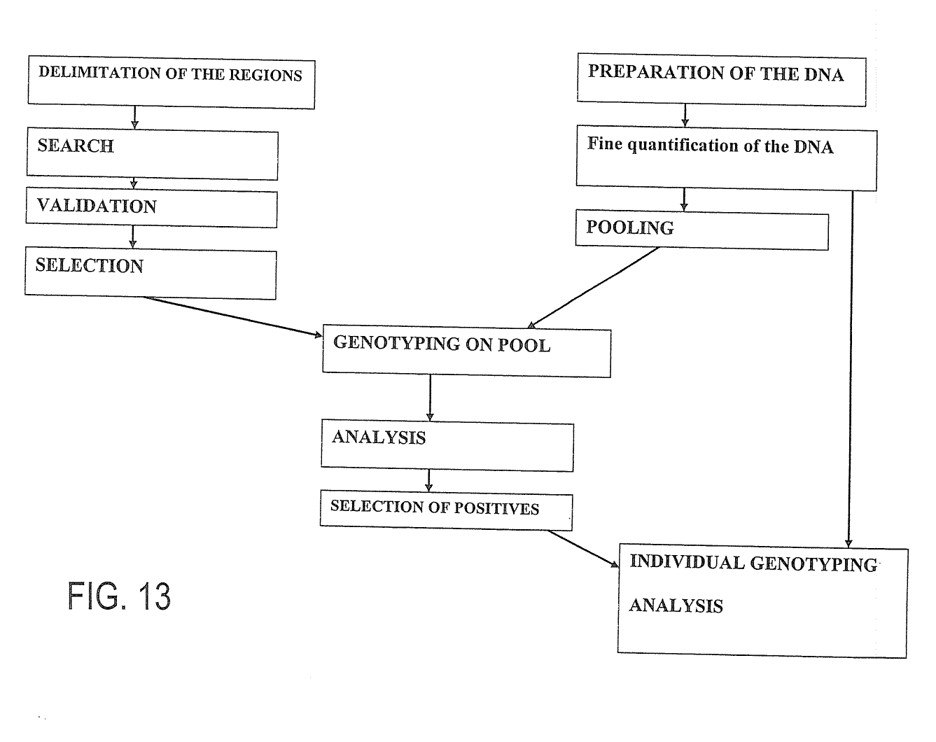

The different steps are described in more detail in the following sections and are shown diagrammatically in FIG. 13.

### 1—Definition of Regions to be Analyzed by SNPs

In a first stage, the inventors more precisely defined the regions of interest on chromosomes 6 and 9, from results obtained from an analysis with microsatellite markers (see Example 1) for the 12 selected families (see Table 6).

The region of chromosome 6 denoted region A was defined by its chromosomal position and by three other types of coordinates for optimum precision and security in defining this region for the subsequent steps. The same was true for the region on chromosome 9 denoted region B.

Region A: chromosomal position: 6p22-6p12.3
Between the HLA-F gene and the microsatellite marker D6S1651
Between SNP rs2075682 and SNP rs1973934
Between positions 39,625,529 bp* and 50,602,544 bp* * The position of the sequence (in terms of base pairs bp) is expressed as a function of the version of the database on the human genome published in December 2001 (i.e. NCBI Build 28).
   Region B: chromosomal position: 9q34.13-9q34.3 (qter)
Between the D9S290 marker and the 9q telomere
Between SNP rs2096071 and SNP rs1378955
Between positions 123,405,258 bp* and 133,021,490 bp* * The position of the sequence (in terms of base pairs bp) is expressed as a function of the version of the database on the human genome published in December 2001 (i.e. NCBI Build 28).

### 2—Investigation of SNP Candidates (in Silico) and Validation (Experimental)

Starting from regions A and B as defined above, a second step consisted of determining a collection of SNPs belonging to these regions to obtain a marker map for the two regions. These markers were also defined so that they covered the 21 Mbp (total length of the two regions) homogeneously and equidistantly. The distance between the different SNPs was fixed to an average of 30 kb. This operation was carried out by selecting 1500 SNPs satisfying these criteria (in silico SNP candidates).

Of the 1500 preselected during the first step, 1379 SNPs proved to be operational. The term “operational” means amplifiable using reagents defined in the usual manner. The 1379 SNPs selected were analyzed over the 92 control individuals (individuals from the Centre d'Etude du Polymorphisme Humain—Center for the study of human polymorphism) to validate the presence of at least two alleles for each SNP (polymorphism validation).

At the end of this experimental selection, only SNPs with an allelic frequency for the rarest allele of at least 10% were retained. Using this method, 697 SNPs were validated, 465 on region A and 232 on region B.

### 3—DNA Pooling

In order to increase the genotyping capacity, a pooling strategy was carried out on the different DNAs. The power of this method has been recorded in various publications (in particular Werner et al, Hum Mutat 2002 July; 20(1):57-64, Bansal et al, Proc Natl Acad Sci USA 2002, Dec. 24; 99(26): 16871-4).

To carry out pooling, DNA was assembled from different individuals with the “premature canities” trait (PC) and from control individuals. Pooling was carried out so that each of the DNA samples was represented in an equimolar manner, to guarantee that no individual would have a preponderant influence on the results. To this end, the exact concentration of each DNA sample was measured using the “picogreen” method in the various samples from individuals.

Groups were constituted taking into account a “phenotype score of canities intensity” which was attributed to each individual as follows.

Firstly, two sorts of criteria were defined, primary criteria to which score values of 2 were assigned, and secondary criteria to which score values of 1 were assigned.

There were 2 primary criteria (score value=2 for each), namely: (i) first white hairs below 18 years; (ii) fair pepper and salt hair at 30.

There were 3 secondary criteria (score value=1 for each), namely: (i) first white hairs below 25 years; (ii) dark pepper and salt hair at 30 years; (iii) family notion of premature canities.

Adding the scores for each individual with each of the diagnostic criteria meant that an intensity score for the premature canities phenotype could be assigned to each individual.

It was also possible to define several different groups depending on the phenotype score. Of the affected individuals, 72 individuals had a score of 4 or 5 or more and 132 individuals had a phenotype score of 2 or more.

Group AI: this group was constituted by DNA from 72 PC individuals with a phenotype score of 4 or 5.
Group AII: this group was constituted by DNA from 132 PC individuals with a phenotype score of 2, 3, 4 or 5.
Groups BI and BII: these groups were constituted by DNA from control individuals with a geographical origin close to that of the PC individuals. For these control individuals, the criteria for selection were: (i) age over 40 years; (ii) no signs of canities in the control individual; (iii) absence of family notion of canities. The criteria for pairing with an individual from group AI or All were identical geographical origin, same sex and identical hair color at 18 years of age.

In this manner, in addition to affected versus unaffected pairing by the PC phenotype, each PC individual from group AI was represented by a control individual in group BI with a close or identical geographical origin. This was the same for each individual in group AII.

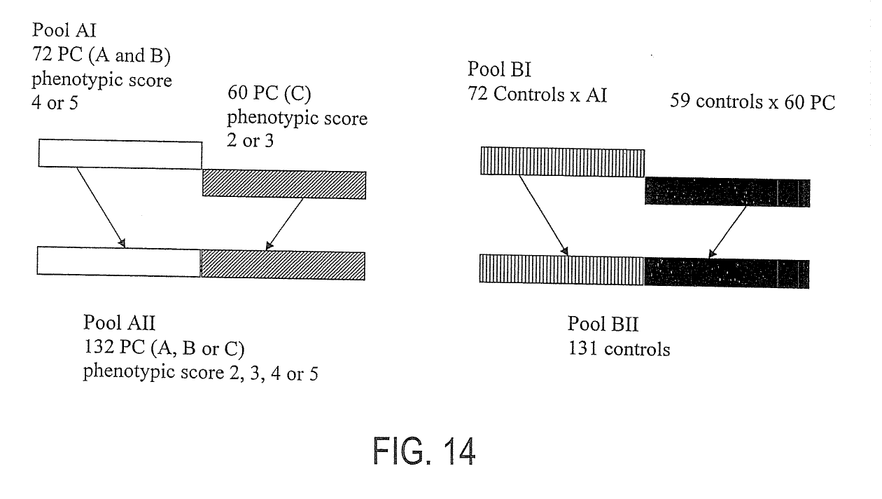

The constitution of the different groups is shown diagrammatically in FIG. 14.

The use of these rigorous methods for clinical diagnosis of affected and control subjects guaranteed the reliability of the quality of the phenotype data.

Further, the rigor of pairing using the miles fixed by the inventors was a guarantee of the pertinence of the statistical analyses comparing the genomic data from these individuals whether grouped into pools or compared individually.

### 4—Selection of Validated SNPs for Genotyping on Grouped DNA

Of the 697 SNPs validated in step 2, 450 were selected in a new selection step. This new selection was based on the interval between the SNPs, fixed as an average of 30 to 50 kb.

The 450 SNPs were defined as follows:

Region A: 283 SNPs

Region B: 167 SNPs

The different SNPs used for the successive steps are shown in the tables below. These tables also include 9 additional SNPs, which were added in a subsequent step to complete the list. These additional 9 SNPs are SNPs n's 19, 38, 103, 105 and 287 for region A and SNPs 86, 97, 131 and 137 for region B.

The 288 SNPs of region A and 171 SNPs of region B are numbered in order of increasing length (telomere p towards telomere q) of regions A and B which they cover quasi-equidistantly and homogeneously.

Region A:

| SNP | |
|---|---|
| N^{o} | Identifier |
| 1 | rs1610602 |
| 2 | rs1737071 |
| 3 | rs1737006 |
| 4 | rs1736936 |
| 5 | 2734988 |
| 6 | 2734967 |
| 7 | 2524033 |
| 8 | ucla34k_818417 |
| 9 | rs426483 |
| 10 | rs259926 |
| 11 | rs259919 |
| 12 | rs1264708 |
| 13 | 1015465 |
| 14 | rs757259 |
| 15 | rs1029237 |
| 16 | rs971570 |
| 17 | rs962899 |
| 18 | rs1045251 |
| 19 | rs261943 |
| 20 | rs1264585 |
| 21 | 984802 |
| 22 | 1245219 |
| 23 | rs1264562 |
| 24 | rs1264513 |
| 25 | rs1059510 |
| 26 | rs1362119 |
| 27 | rs1110465 |
| 28 | rs1264432 |
| 29 | rs1264420 |
| 30 | 1076829 |
| 31 | 1075496 |
| 32 | 51457 |
| 33 | 2535323 |
| 34 | rs1264377 |
| 35 | rs1264347 |
| 36 | 2535326 |
| 37 | rs1264326 |
| 38 | rs1419693 |
| 39 | rs1264300 |
| 40 | 2532921 |
| 41 | rs1634713 |
| 42 | 1419664 |
| 43 | 2517502 |
| 44 | 2535291 |
| 45 | 2284177 |
| 46 | rs1265111 |
| 47 | rs1265181 |
| 48 | rs1639108 |
| 49 | 1793891 |
| 50 | rs1819788 |
| 51 | rs1005248 |
| 52 | rs1620583 |
| 53 | 2596429 |
| 54 | 2507977 |
| 55 | 2516446 |
| 56 | 2523675 |
| 57 | rs1065076 |
| 58 | 2857605 |
| 59 | 239157 |
| 60 | 2736176 |
| 61 | rs805303 |
| 62 | rs805282 |
| 63 | rs805293 |
| 64 | rs707939 |
| 65 | rs743399 |
| 66 | rs733539 |
| 67 | rs494620 |
| 68 | rs644045 |
| 69 | ucla34k_328681 |
| 70 | 1265899 |
| 71 | rs1150755 |
| 72 | 204999 |
| 73 | rs1061808 |
| 74 | rs1044506 |
| 75 | rs367398 |
| 76 | rs397379 |
| 77 | rs482194 |
| 78 | rs505274 |
| 79 | rs1265758 |
| 80 | 1555115 |
| 81 | rs743862 |
| 82 | rs983561 |
| 83 | rs1548306 |
| 84 | 2308818 |
| 85 | rs1987948 |
| 86 | 608766 |
| 87 | 60071 |
| 88 | rs763024 |
| 89 | rs763028 |
| 90 | 2857152 |
| 91 | 241455 |
| 92 | 2621426 |
| 93 | rs241405 |
| 94 | 241398 |
| 95 | 154973 |
| 96 | 206779 |
| 97 | 2856817 |
| 98 | rs1883414 |
| 99 | rs721393 |
| 100 | rs1799908 |
| 101 | rs439205 |
| 102 | rs213194 |
| 103 | rs462618 |
| 104 | rs213201 |
| 105 | rs462093 |
| 106 | rs1014779 |
| 107 | rs211467 |
| 108 | rs211457 |
| 109 | 381847 |
| 110 | rs1755047 |
| 111 | 769051 |
| 112 | rs396516 |
| 113 | rs210145 |
| 114 | rs494835 |
| 115 | rs1570760 |
| 116 | rs943470 |
| 117 | rs498114 |
| 118 | rs652049 |
| 119 | rs943475 |
| 120 | 942496 |
| 121 | 2104362 |
| 122 | rs1853656 |
| 123 | 747889 |
| 124 | 2499740 |
| 125 | 2495975 |
| 126 | 902197 |
| 127 | rs733457 |
| 128 | rs2029461 |
| 129 | 1776888 |
| 130 | 1759627 |
| 131 | rs206942 |
| 132 | rs206930 |
| 133 | rs206919 |
| 134 | 2395560 |
| 135 | rs205284 |
| 136 | 2744971 |
| 137 | 2814986 |
| 138 | rs2064253 |
| 139 | 2814951 |
| 140 | rs912716 |
| 141 | rs1555773 |
| 142 | 2395607 |
| 143 | 2296362 |
| 144 | rs1051115 |
| 145 | rs847852 |
| 146 | rs847846 |
| 147 | rs707967 |
| 148 | rs1886243 |
| 149 | rs942373 |
| 150 | rs1888822 |
| 151 | 2071920 |
| 152 | 2267663 |
| 153 | rs1888823 |
| 154 | 2267664 |
| 155 | rs2038067 |
| 156 | 2894401 |
| 157 | rs1016146 |
| 158 | rs2064319 |
| 159 | 2395634 |
| 160 | rs1360780 |
| 161 | 2766532 |
| 162 | 2766557 |
| 163 | rs1998894 |
| 164 | rs879668 |
| 165 | rs1049649 |
| 166 | 2395639 |
| 167 | 2250151 |
| 168 | rs651158 |
| 169 | rs969659 |
| 170 | rs743923 |
| 171 | rs851016 |
| 172 | rs851007 |
| 173 | rs743926 |
| 174 | 2859129 |
| 175 | 2245972 |
| 176 | 1029312 |
| 177 | rs1541316 |
| 178 | rs933234 |
| 179 | rs763021 |
| 180 | 2071794 |
| 181 | rs941816 |
| 182 | rs664370 |
| 183 | rs1061632 |
| 184 | rs605684 |
| 185 | rs648125 |
| 186 | rs1406945 |
| 187 | rs720170 |
| 188 | 2395656 |
| 189 | rs236470 |
| 190 | rs236430 |
| 191 | rs236402 |
| 192 | rs236375 |
| 193 | 625474 |
| 194 | 449840 |
| 195 | rs8472 |
| 196 | 707542 |
| 197 | 831477 |
| 198 | 2567280 |
| 199 | 2734977 |
| 200 | 445117 |
| 201 | 1565356 |
| 202 | 2252937 |
| 203 | 2596464 |
| 204 | 2442750 |
| 205 | 1041523 |
| 206 | 1041524 |
| 207 | 693955 |
| 208 | 483536 |
| 209 | ucla34k_654528 |
| 210 | 2396240 |
| 211 | 2221224 |
| 212 | 1331293 |
| 213 | 545455 |
| 214 | 911983 |
| 215 | 2025230 |
| 216 | 1322651 |
| 217 | 326799 |
| 218 | 1384546 |
| 219 | 477011 |
| 220 | 227813 |
| 221 | 609699 |
| 222 | 1338908 |
| 223 | 1557143 |
| 224 | 1293467 |
| 225 | rs542444 |
| 226 | 626965 |
| 227 | 857318 |
| 228 | 636845 |
| 229 | 1329711 |
| 230 | 1329714 |
| 231 | 2396380 |
| 232 | 1285007 |
| 233 | 1748235 |
| 234 | 1284958 |
| 235 | 2763135 |
| 236 | 2024786 |
| 237 | 1321081 |
| 238 | rs1321076 |
| 239 | 1343799 |
| 240 | 1934328 |
| 241 | 1928533 |
| 242 | 713270 |
| 243 | 1449648 |
| 244 | 1449642 |
| 245 | 1375696 |
| 246 | 756081 |
| 247 | 3088356 |
| 248 | 2095771 |
| 249 | 1555214 |
| 250 | 1338471 |
| 251 | 2179994 |
| 252 | 1555215 |
| 253 | rs1204296 |
| 254 | 995564 |
| 255 | ucla34k_299503 |
| 256 | 2799353 |
| 257 | 1442224 |
| 258 | 1899405 |
| 259 | 2396635 |
| 260 | 2277121 |
| 261 | 1867015 |
| 262 | 953887 |
| 263 | 1527707 |
| 264 | 871728 |
| 265 | 1881030 |
| 266 | 1234181 |
| 267 | 699945 |
| 268 | 2206927 |
| 269 | 952884 |
| 270 | 2216464 |
| 271 | 1421372 |
| 272 | 1862008 |
| 273 | 1410820 |
| 274 | 1410825 |
| 275 | ucla34k_810022 |
| 276 | 220669 |
| 277 | 220711 |
| 278 | 2021916 |
| 279 | 1932033 |
| 280 | 1226490 |
| 281 | 2171937 |
| 282 | 819511 |
| 283 | 937054 |
| 284 | 926774 |
| 285 | 1986278 |
| 286 | 2207224 |
| 287 | 2281458 |
| 288 | 993612 |

Region B

| SNP | |
|---|---|
| N^{o} | Identifier |
| 1 | 2096071 |
| 2 | 2282394 |
| 3 | 2805103 |
| 4 | 1331336 |
| 5 | 1533967 |
| 6 | 2282179 |
| 7 | 2011978 |
| 8 | 955910 |
| 9 | 1147360 |
| 10 | rs940373 |
| 11 | 2498905 |
| 12 | 2542248 |
| 13 | 1220653 |
| 14 | 1867099 |
| 15 | ucla34k_454177 |
| 16 | 2241271 |
| 17 | 1017509 |
| 18 | rs1182 |
| 19 | rs732074 |
| 20 | rs1125962 |
| 21 | ucla34k_598296 |
| 22 | 1322671 |
| 23 | 1570381 |
| 24 | rs676492 |
| 25 | 2286792 |
| 26 | 53558 |
| 27 | 1860641 |
| 28 | 885345 |
| 29 | rs1043368 |
| 30 | 1557126 |
| 31 | 947507 |
| 32 | 914977 |
| 33 | 2210623 |
| 34 | 1475731 |
| 35 | 928518 |
| 36 | 1864709 |
| 37 | 944605 |
| 38 | 2304812 |
| 39 | 1866974 |
| 40 | 2269337 |
| 41 | 2583839 |
| 42 | 2791743 |
| 43 | 2855181 |
| 44 | 2987903 |
| 45 | 2314027 |
| 46 | 1544012 |
| 47 | 1997242 |
| 48 | 928677 |
| 49 | 928678 |
| 50 | 2315073 |
| 51 | 933093 |
| 52 | 2315076 |
| 53 | 2315078 |
| 54 | 981759 |
| 55 | 2483469 |
| 56 | 2478858 |
| 57 | 2966373 |
| 58 | 540621 |
| 59 | 2994056 |
| 60 | 2275500 |
| 61 | 10K-56700 |
| 62 | rs943851 |
| 63 | 2282006 |
| 64 | 1887786 |
| 65 | 2076 |
| 66 | 928013 |
| 67 | 869381 |
| 68 | 3012757 |
| 69 | 2987378 |
| 70 | 3012717 |
| 71 | 1331631 |
| 72 | 1412075 |
| 73 | 1331625 |
| 74 | 2149171 |
| 75 | ucla34k_694625 |
| 76 | 2296868 |
| 77 | rs1185193 |
| 78 | 10K-52978 |
| 79 | 563521 |
| 80 | 507998 |
| 81 | 2362369 |
| 82 | 577416 |
| 83 | 944812 |
| 84 | rs1470190 |
| 85 | 2247393 |
| 86 | 418620 |
| 87 | 787469 |
| 88 | rs302919 |
| 89 | 913705 |
| 90 | 932886 |
| 91 | 429269 |
| 92 | 2526008 |
| 93 | 2072058 |
| 94 | rs739441 |
| 95 | 2905078 |
| 96 | 64967 |
| 97 | 2905179 |
| 98 | rs649168 |
| 99 | 645841 |
| 100 | rs644234 |
| 101 | 532861 |
| 102 | 59071 |
| 103 | 1179040 |
| 104 | 1887519 |
| 105 | 1179001 |
| 106 | ucla34k_576465 |
| 107 | 954052 |
| 108 | 2492057 |
| 109 | 2506715 |
| 110 | 2506696 |
| 111 | 1079783 |
| 112 | rs77905 |
| 113 | 129891 |
| 114 | 2027963 |
| 115 | 628936 |
| 116 | rs602990 |
| 117 | 2428091 |
| 118 | 2428123 |
| 119 | 2519770 |
| 120 | 2428083 |
| 121 | 2789861 |
| 122 | 414848 |
| 123 | 1536474 |
| 124 | 943435 |
| 125 | 943429 |
| 126 | 2182640 |
| 127 | ucla34k_177347 |
| 128 | 16832 |
| 129 | ucla34k_642641 |
| 130 | 2989736 |
| 131 | 2989728 |
| 132 | 3012797 |
| 133 | 1038193 |
| 134 | 2279265 |
| 135 | 964138 |
| 136 | 515078 |
| 137 | 484397 |
| 138 | 518630 |
| 139 | 752835 |
| 140 | 1778993 |
| 141 | 1891996 |
| 142 | 1106256 |
| 143 | 2382867 |
| 144 | 2065385 |
| 145 | 872667 |
| 146 | 914400 |
| 147 | ucla34k_923462 |
| 148 | 1412512 |
| 149 | rs968569 |
| 150 | 210086 |
| 151 | 783770 |
| 152 | 872006 |
| 153 | 1537414 |
| 154 | 574840 |
| 155 | 1001523 |
| 156 | 755722 |
| 157 | 1318383 |
| 158 | 730399 |
| 159 | 1009473 |
| 160 | 47713 |
| 161 | 2297690 |
| 162 | 2139881 |
| 163 | 1335099 |
| 164 | 55096 |
| 165 | 2501566 |
| 166 | 2501559 |
| 167 | 2183138 |
| 168 | 1054864 |
| 169 | 2275781 |
| 170 | 1891629 |
| 171 | 1099298 |

### 5—Genotyping Pooled DNA

For the 459 SNPs retained during step 4, the subsequent step was to determine their allelotype, i.e. the frequency of each of the alleles, for the 4 groups of pooled DNA depending on the severity and premature nature of the phenotype (see the definition of the 4 groups in step 3 and FIG. 14).

The allelic frequency of the two alleles was determined for each of the SNPs in the 4 groups. The statistical significance of the differences in allelic frequencies between groups AI and BI or AII and BII was estimated by the “p” value representing the significance. The lower the p value, the more statistically significant the distance.

The experiments were repeated 3 times (3 PCR), each of the three PCRs then being tested 5 times using MALDI-TOF to obtain a reliable mean value.

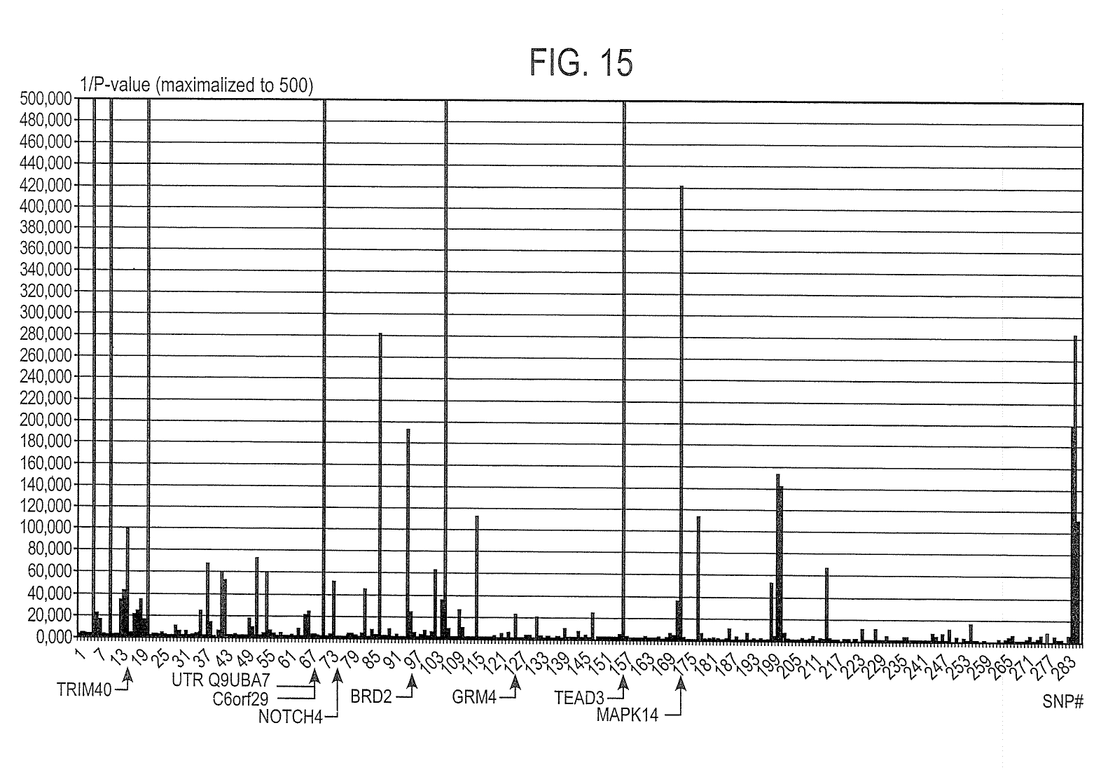

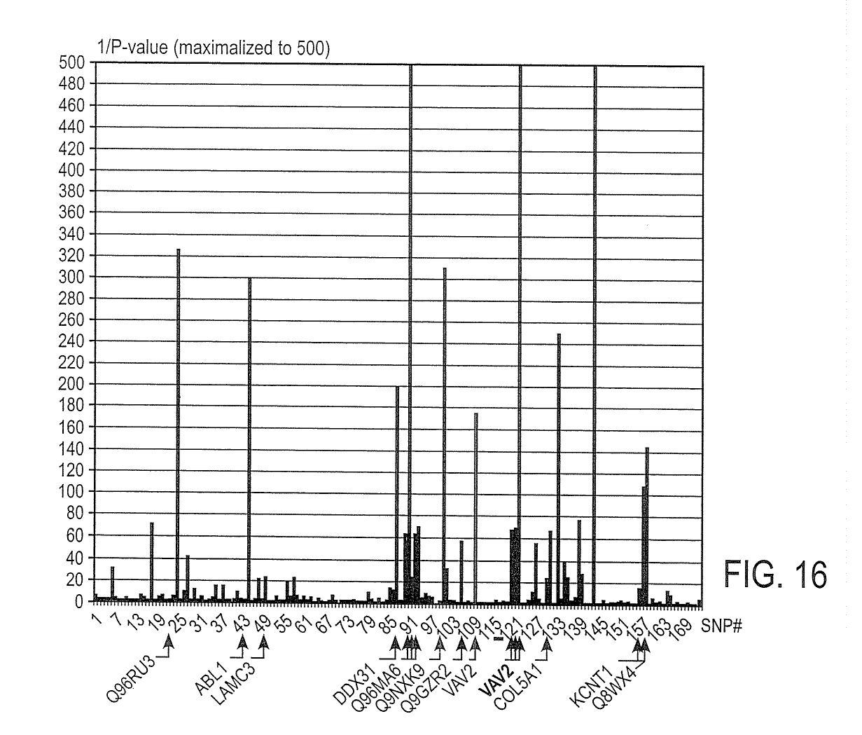

FIGS. 15 and 16 illustrate the results obtained for regions A and B respectively for each SNP (numbered from 1 to 288 along region A and from 1 to 171 along region B). The ordinate shows l/p but values of more than 500 (i.e. p<0.002) were maximized to 500. Table 17 summarizes the results obtained:

TABLE 17
genotyping pools, number of positive SNPs (total 74).
chromosome 6

| AI − BI < 0.05 and AII − BII < 0.05 | AI − BI < 0.05 | AII − BII < 0.05 |
|---|---|---|
| 11 | 20 | 10 |

chromosome 9

| | | |
|---|---|---|
| AI − BI < 0.05 and AII − BII < 0.05 | AI − BI < 0.05 | AII − BII < 0.05 |
| 2 | 9 | 22 |

These results show the existence of clusters, i.e. at least three consecutive SNPs (i.e. physically close to each other on the human genome) which all have a significance p of less than 0.05 (termed “positive SNPs”). Some of these clusters are shown in FIGS. 15 and 16.

Table 18 summarizes the various features in the distribution of SNPs in regions A and B.

TABLE 18
Distribution features of positive SNPs in regions A and B.
chromosome 6

| | |
|---|---|
| clusters (3 or more positive consecutive SNPs) | 4 |
| pairs (2 positive consecutive SNPs) | 5 |
| double spots (2 positive SNPs separated by a negative SNP) | 2 |

chromosome 9

| | |
|---|---|
| clusters (3 or more positive consecutive SNPs) | 2 |
| pairs (2 positive consecutive SNPs) | 4 |
| double spots (2 positive SNPs separated by a negative SNP) | 2 |

The different genes of regions A and B which were identified by positive SNPs distributed in clusters, isolated or in double spots constitute a first series of candidate genes, including the predicted genes. The list is as follows:

### Region A:

RNF9, TRIM15, TRIM26, RNF23, FLJ22638, DDR1, HLA-B, HLA-DMB, HLA-DMA, COL11A2, SACM2L, RPS18, B3GALT4, HKE2, RAB2L, TAPBF, ZNF297, DAXX, MAPK14, DOM3Z, MICA, LOC51323, TNFRSF21, MICA, HSPA1B, TNXB, CYP21A2, NOTCH₄, PBX2, HLA-DRA, PHF1, ITPR3, MGC14833, BAK1, 1HPK3, GRM4, TCP11, TEAD3

### Region B

DDX31, GTF3C4, C9ORF9, TSC1, ABL1, LOC57109, FREQ, ADAMTS13, LAMC3, SURF5, SURF6, FCN2, FCN1, OLFM1, VAV2, ABO, CELL, SARDH.

A more detailed analysis was carried out which produced a new list of genes overlapped by a positive SNP, using ENSEMBL (ENSEMBL v.8.30a. 117, September 2002). This list included the genes (coding, untranslated region UTR, and intronic) overlapped by a positive SNP, excluding genes which were close to a positive SNP located in a regulating region.

### Coding:

chromosome 6: TRM40, C6ORF29, NOTCH₄

### UTR:

chromosome 6: □9UBA7

### Intronic:

### Chromosome 6: BRD2, GRM4, TEAD3, MAPK14

### Chromosome 9: Q96RU3, ABL1, LAMC3, Q96MA6, Q9NXK9, Q9GZR2, VAV2, COL5A1, KCNT1, Q8WX41

A new analysis for the predicted genes using ENSEMBL gave the following results:

Chromosome 6: ENST00000259854, ENST00000259855, ENST00000259941, ENST00000259940, ENST00000259930, ENST00000274855, ENST00000259847, ENST000002935B7, ENST00000299124, ENST00000259945, ENST00000259862, ENST00000259876, ENST00000259875, ENST00000259895, ENST00000293682, ENST00000229412, ENST00000229725, ENST00000229729, ENST00000229825, ENST00000244501, ENST00000293728, ENST00000244371, ENST00000293739, ENST00000230240, ENST00000211372, ENST00000244475, ENST00000266008, ENST00000230251, ENST00000244369, ENST00000299851, ENST00000230255, ENST00000229795, ENST00000229794, ENST00000296861, ENST00000274795, ENST0000029364.5, ENST00000293707, ENST00000229780, ENST00000299791, ENST00000293720, ENST00000244411, ENST00000266007, ENST00000229422.
Chromosome 9: ENST00000298489, ENST00000266097, ENST00000263612, ENST00000245590, ENST00000298545, ENST00000298546, ENST00000298552, ENST00000298554, ENST00000298555, ENST00000277434, ENST00000277433, ENST00000298632, ENST00000291687, ENST00000298656, ENST00000298658, ENST00000298660, ENST00000277355, ENST00000298678, ENST00000298676, ENST00000298656, ENST00000298658, ENST00000298660, ENST00000277355, ENST00000298678, ENST00000298676, ENST00000298682, ENST00000298683, ENST00000291744, ENST00000291741, ENST00000223427, ENST00000198253, ENST00000277527, ENST00000263604, ENST00000266109, ENST00000298467, ENST00000266100, ENST00000277422, ENST00000263609.

The tables below record the predicted genes in regions A and B in the clusters, double spots (DS) and individual positive SNPs starting from NCBI Build 28 (December 2001) “CDS” indicates the coding sequence and “tx” indicates the transcript.

REGION A

| SNP# | NAME | chrom | cdsStart | cdsEnd | txStart | txEnd | Strand | Nb EXONS |
|---|---|---|---|---|---|---|---|---|
| 5 to 6 | ENST00000259854 | chr6 | 39694455 | 39909459 | 39694455 | 39909459 | + | 7 |
| | ENST00000259855 | chr6 | 39730113 | 39909459 | 39730113 | 39909459 | + | 7 |
| 13 to 21 (cluster grouping) | ENST00000259941 | chr6 | 40054160 | 40061326 | 40052414 | 40061402 | − | 8 |
| | ENST00000259940 | chr6 | 40054447 | 40061281 | 40053956 | 40061402 | − | 5 |
| | ENST00000259930 | chr6 | 40064144 | 40072806 | 40063675 | 40073156 | + | 7 |
| | ENST00000274855 | chr6 | 40064144 | 40073064 | 40064144 | 40073064 | + | 2 |
| | ENST00000259847 | chr6 | 40086320 | 40099537 | 40085023 | 40105196 | − | 9 |
| | ENST00000293587 | chr6 | 40160063 | 40162772 | 40160063 | 40162772 | + | 6 |
| | ENST00000299124 | chr6 | 40229717 | 40242662 | 40228052 | 40244119 | + | 8 |
| | ENST00000259945 | chr6 | 40229717 | 40242662 | 40228052 | 40244119 | + | 9 |
| | ENST00000259862 | chr6 | 40245577 | 40247213 | 40245564 | 40247263 | + | 5 |
| 18 to 21 DS | ENST00000299124 | chr6 | 40229717 | 40242662 | 40228052 | 40244119 | + | 8 |
| | ENST00000259945 | chr6 | 40229717 | 40242662 | 40228052 | 40244119 | + | 9 |
| | ENST00000259862 | chr6 | 40245577 | 40247213 | 40245564 | 40247263 | + | 5 |
| 36 to 38 DS | ENST00000259876 | chr6 | 40789238 | 40799804 | 40784592 | 40800667 | + | 20 |
| | ENST00000259875 | chr6 | 40789238 | 40799804 | 40784592 | 40800667 | + | 19 |
| | ENST00000259895 | chr6 | 40809542 | 40814486 | 40808733 | 40814609 | + | 14 |
| 66 to 67 | ENST00000293682 | chr6 | 41735883 | 41738214 | 41733465 | 41738312 | + | 4 |
| | ENST00000229412 | chr6 | 41733505 | 41735914 | 41733465 | 41738312 | + | 4 |
| | ENST00000229725 | chr6 | 41758412 | 41761467 | 41757893 | 41761597 | − | 6 |
| | ENST00000229729 | chr6 | 41762320 | 41777667 | 41762320 | 41777667 | − | 21 |
| 95 to 96 | ENST00000229825 | chr6 | 42776521 | 42781987 | 42775815 | 42782220 | − | 6 |
| | ENST00000244501 | chr6 | 42790440 | 42794203 | 42789785 | 42794258 | − | 5 |
| | ENST00000293728 | chr6 | 42790440 | 42793704 | 42790440 | 42793704 | − | 4 |
| | ENST00000244371 | chr6 | 42814079 | 42821890 | 42809841 | 42822479 | + | 13 |
| 103 to 106 DS | ENST00000293739 | chr6 | 43091805 | 43112637 | 43091802 | 43112637 | − | 20 |
| | ENST00000230240 | chr6 | 43091805 | 43112637 | 43091802 | 43112637 | − | 18 |
| | ENST00000211372 | chr6 | 43113589 | 43117425 | 43113036 | 43117466 | + | 6 |
| | ENST00000244475 | chr6 | 43118381 | 43119515 | 43118381 | 43119515 | + | 1 |
| | ENST00000266008 | chr6 | 43120240 | 43130132 | 43120066 | 43130176 | − | 15 |
| | ENST00000230251 | chr6 | 43130818 | 43131804 | 43130818 | 43131804 | + | 4 |
| | ENST00000244369 | chr6 | 43133066 | 43139572 | 43132620 | 43139923 | − | 18 |
| | ENST00000299851 | chr6 | 43136980 | 43138945 | 43136980 | 43138945 | − | 6 |
| | ENST00000230255 | chr6 | 43144915 | 43155002 | 43142556 | 43155173 | − | 8 |
| 172 to 173 | ENST00000229795 | chr6 | 45869083 | 45949370 | 45868721 | 45951684 | + | 12 |
| | ENST00000229794 | chr6 | 45869083 | 45949370 | 45868721 | 45951684 | + | 12 |
| 285 to 286 | ENST00000296861 | chr6 | 50493533 | 50493905 | 50493248 | 50548202 | − | 5 |
| | ENST00000274795 | chr6 | 50494484 | 50571228 | 50494205 | 50571622 | − | 6 |
| | SNP + individuals | | | | | | | |
| 10 | no genes | | | | | | | |
| 52 | no genes | | | | | | | |
| 55 | ENST00000293645 | chr6 | 41309005 | 41405874 | 41309005 | 41405874 | + | 3 |
| 71 | ENST00000293707 | chr6 | 41935275 | 41963926 | 41935275 | 41963926 | − | 15 |
| 74 | ENST00000229780 | chr6 | 42061138 | 42089652 | 42061115 | 42089727 | − | 31 |
| 83 | no genes | | | | | | | |
| 87 | no genes | | | | | | | |
| 89 | ENST00000299791 | chr6 | 42501668 | 42604749 | 42501668 | 42604749 | − | 5 |
| | ENST00000293720 | chr6 | 42501668 | 42658154 | 42501668 | 42658154 | − | 4 |
| 99 | no genes | | | | | | | |
| 110 | no genes | | | | | | | |
| 115 | ENST00000244411 | chr6 | 43462531 | 43536706 | 43462495 | 43537491 | + | 58 |
| 126 | ENST00000266007 | chr6 | 43863752 | 43974425 | 43862779 | 43974595 | − | 10 |
| 148 | no genes | | | | | | | |
| 157 | ENST00000229422 | chr6 | 45315972 | 45327588 | 45314555 | 45337961 | − | 12 |
| 178 | no genes | | | | | | | |
| 199 | no genes | | | | | | | |
| 201 | no genes | | | | | | | |
| 215 | no genes | | | | | | | |
| 226 | no genes | | | | | | | |

REGION B

| SNP# | NAME | chrom | cdsStart | cdsEnd | txStart | txEnd | Strand | Nb EXONS |
|---|---|---|---|---|---|---|---|---|
| 47 to 49 DS | ENST00000298489 | chr9 | 125457741 | 125470257 | 125373136 | 125470567 | + | 28 |
| | ENST00000266097 | chr9 | 125373234 | 125470257 | 125373136 | 125470567 | + | 28 |
| 86 to 99 DS chr9: | ENST00000263612 | chr9 | 127045062 | 127120443 | 127044482 | 127120595 | − | 20 |
| 127094511-127505542 | ENST00000245590 | chr9 | 127120792 | 127139136 | 127120534 | 127139622 | + | 5 |
| | ENST00000298545 | chr9 | 127175884 | 127328449 | 127175884 | 127328449 | − | 13 |
| | ENST00000298546 | chr9 | 127334141 | 127338631 | 127328556 | 127340224 | + | 4 |
| | ENST00000298552 | chr9 | 127346431 | 127379066 | 127341543 | 127394815 | − | 23 |
| | ENST00000298554 | chr9 | 127436872 | 127441241 | 127436872 | 127441244 | + | 6 |
| | ENST00000298555 | chr9 | 127469679 | 127471065 | 127469615 | 127471372 | + | 1 |
| | ENST00000277434 | chr9 | 127501047 | 127508171 | 127501047 | 127508171 | + | 8 |
| | ENST00000277433 | chr9 | 127481205 | 127508171 | 127480906 | 127508695 | + | 11 |
| 118 to 120 | ENST00000298632 | chr9 | 128877580 | 128878579 | 128877580 | 128878579 | − | 1 |
| | ENST00000291687 | chr9 | 128750384 | 128978689 | 128750384 | 128978689 | − | 27 |
| 137 to 138 chr9: | 0 | | | | | | | |
| 130035429-130045373 | | | | | | | | |
| 128 to 134 DS chr9: | ENST00000298656 | chr9 | 129757553 | 129770881 | 129757553 | 129770881 | − | 16 |
| 129656527-129977399 | ENST00000298658 | chr9 | 129757607 | 129781523 | 129757607 | 129781523 | − | 13 |
| | ENST00000298660 | chr9 | 129757553 | 129786638 | 129757553 | 129786638 | − | 26 |
| | ENST00000277355 | chr9 | 129607564 | 129789067 | 129607564 | 129789067 | + | 29 |
| | ENST00000298678 | chr9 | 129811215 | 129812613 | 129811213 | 129812613 | + | 2 |
| | ENST00000298676 | chr9 | 129814180 | 129826506 | 129607438 | 129826534 | + | 37 |
| | ENST00000298682 | chr9 | 129864608 | 129868564 | 129864598 | 129870351 | + | 5 |
| | ENST00000298683 | chr9 | 129864608 | 129869270 | 129864598 | 129871307 | + | 7 |
| | ENST00000291744 | chr9 | 129864608 | 129871199 | 129864598 | 129871307 | + | 8 |
| | ENST00000291741 | chr9 | 129864608 | 129871199 | 129864598 | 129871307 | + | 7 |
| | ENST00000223427 | chr9 | 129893584 | 129901655 | 129893369 | 129901747 | − | 9 |
| | ENST00000198253 | chr9 | 129896270 | 129901655 | 129893369 | 129901747 | − | 8 |
| 155 to 156 chr9: | ENST00000277527 | chr9 | 130609471 | 130715656 | 130609471 | 130715656 | − | 4 |
| 130714327-130728681 | ENST00000263604 | chr9 | 130691065 | 130775279 | 130691064 | 130775281 | + | 29 |
| | SNP + individuals | | | | | | | |
| 6 | no genes | | | | | | | |
| 17 | no genes | | | | | | | |
| 24 | ENST00000266109 | chr9 | 124213577 | 124360885 | 124213576 | 124360901 | − | 15 |
| 27 | no genes | | | | | | | |
| 44 | ENST00000298467 | chr9 | 125063030 | 125234391 | 125063030 | 125234391 | + | 11 |
| | ENST00000266100 | chr9 | 125184157 | 125234391 | 125183776 | 125236384 | + | 11 |
| 57 | no genes | | | | | | | |
| 100 | no genes | | | | | | | |
| 104 | ENST00000277422 | chr9 | 128045772 | 128056657 | 128044878 | 128056857 | − | 8 |
| 108 | no genes | | | | | | | |
| 125 | ENST00000263609 | chr9 | 129380168 | 129507477 | 129380168 | 129507477 | + | 9 |
| 141 | no genes | | | | | | | |

### 6—Selection of SNPs for Genotyping Individual DNA

Of the 459 SNPs used to genotype pooled DNA, 76 were retained for genotyping individual DNA. The retained SNPs had a statistically significant distance when genotyping the pools, i.e. p<0.05 for AI-BI, AII-BII or AI-BII. Their distribution was as follows

| | | | |
|---|---|---|---|
| Region A: | 43 SNPs | Region B: | 33 SNPs |

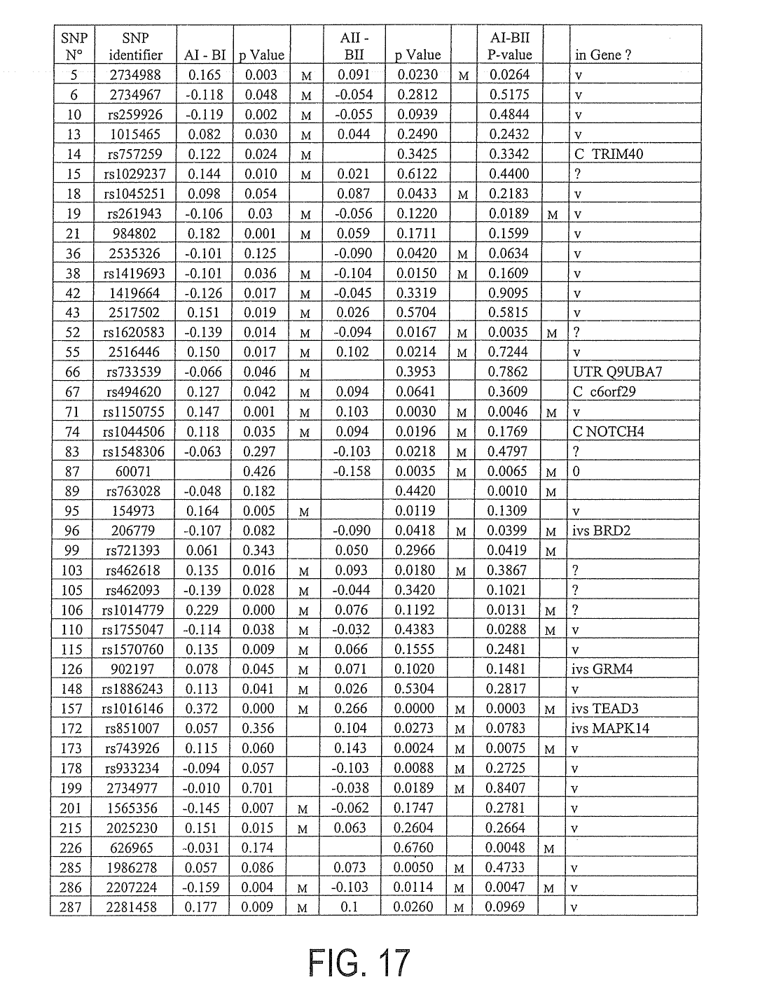

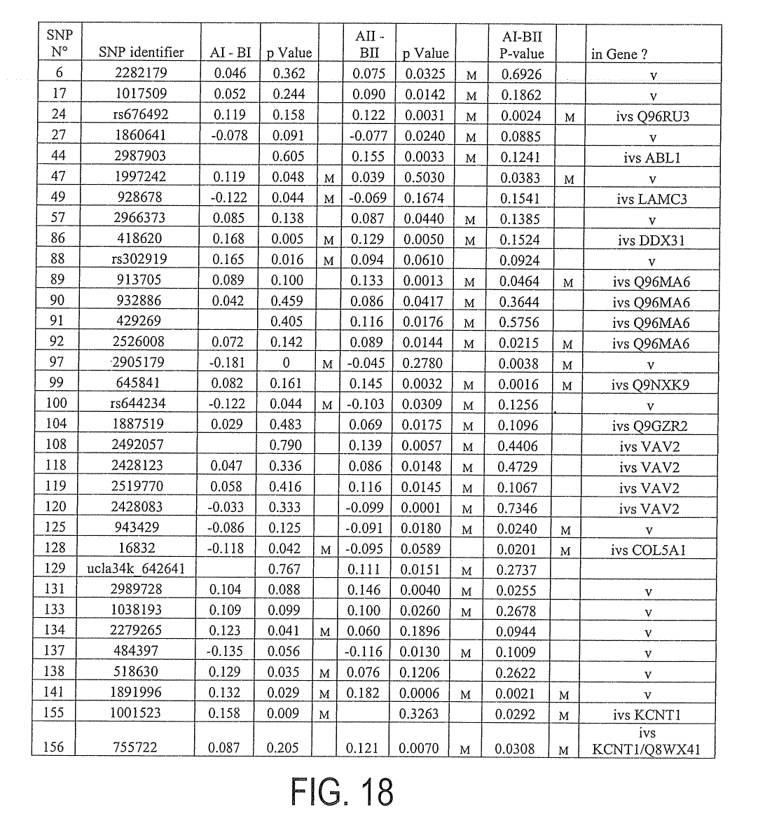

The list of SNP selected and A-B comparison are given in FIG. 17 (region A) and 18 (region B).
Table 19 summarizes the results obtained.

TABLE 19
Choice of positive SNPs (total 76) following results of pool genotyping
chromosome 6

| AI − BI < 0.05 and AII − BII < 0.05 | AI − BI < 0.05 | AII − BII < 0.05 | AI − BII < 0.05 |
|---|---|---|---|
| 10 | 30 | 20 | 13 |

chromosome 9

| | | | |
|---|---|---|---|
| AI − BI < 0.05 and AII − BII < 0.05 | AI − BI < 0.05 | AII − BII < 0.05 | AI − BII < 0.05 |
| 3 | 11 | 25 | 11 |

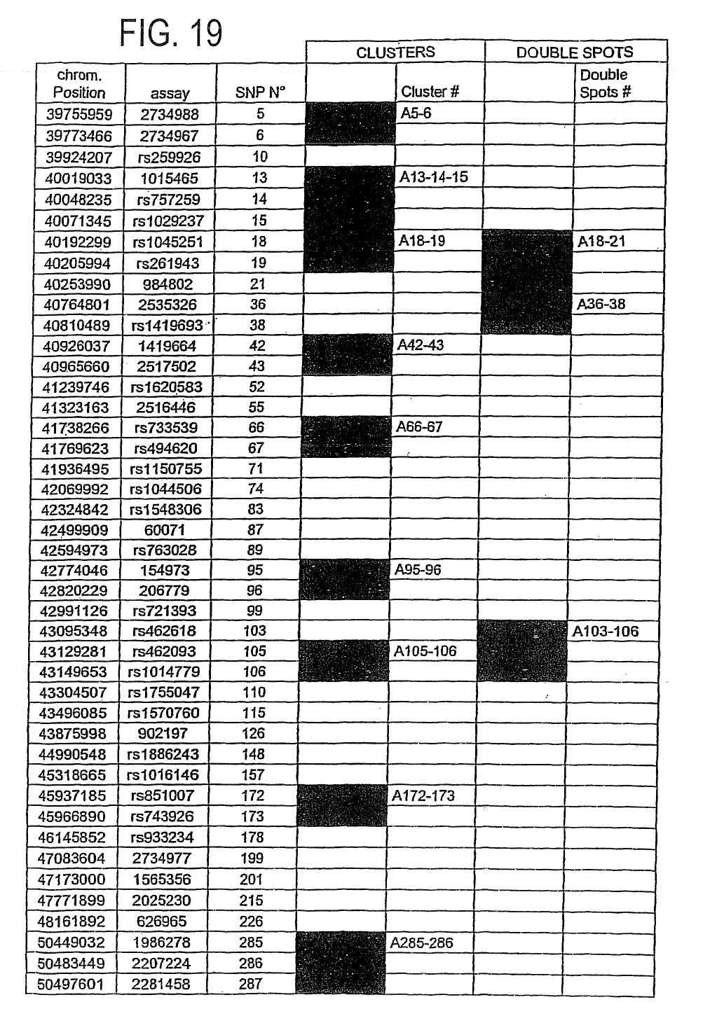

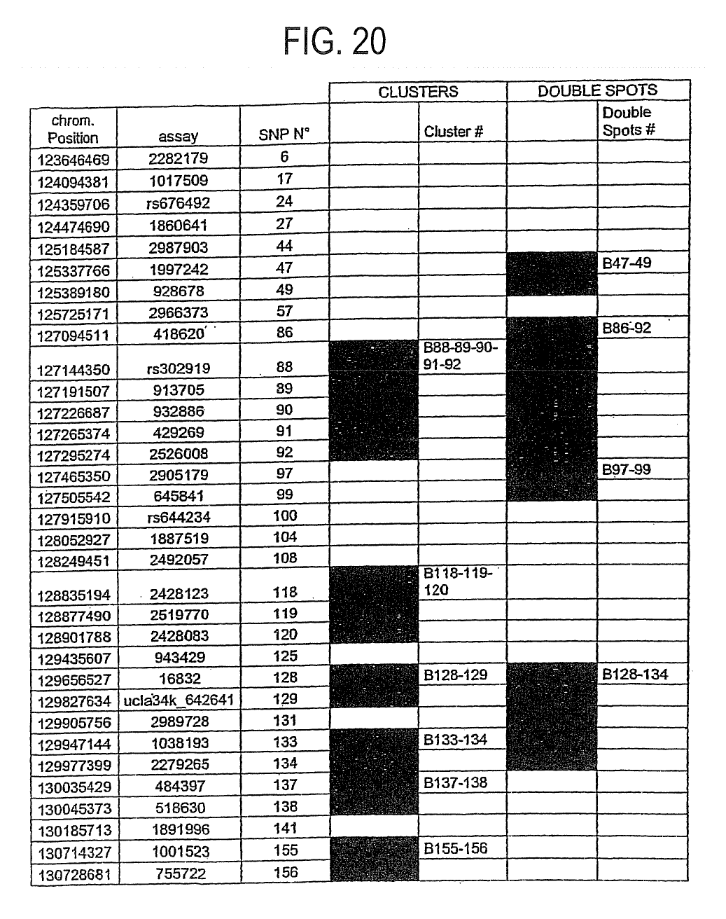

The choice of 76 SNPs for individual genotyping was concentrated on the SNPs present in clusters, those forming pairs (2 consecutive positive SNPs) and those forming double spots (2 positive SNPs separated by a negative SNP). FIGS. 19 and 20 illustrate the distribution of the selected 76 SNPs.

It was observed that the estimate of the allelic frequencies in the pools (and not in individuals) could result in false positives and that this tendency was high when the pools contained less than 200 DNA samples. For this reason, isolated positive SNPs were removed as well as those which were in discord with the controls (BI and BII).

The 76 SNPs were individually analyzed over all available DNAs (187 individuals with the PC phenotype and 186 control individuals with no PC phenotype).

This individual genotyping allowed the allele frequency and genotype frequency observed in the different groups to be calculated accurately. These data also allowed the distribution of haplotypes observed in positive SNPs organized into clusters to be compared. The term “haplotype” means the combination of alleles tending to be transmitted together.

Integrated analysis of these data allowed SNPs or groups of SNPs to be determined which showed an association with the PC trait, i.e. an allele or a set of alleles which, in a population, are transmitted most frequently with this trait.

### 7—Linkage Disequilibrium Study

Linkage disequilibrium (LD) was analyzed using the GenePop program in the absence of data on the phase of the haplotypes on the analyzed chromosomes.

Linkage disequilibrium is a situation in which 2 genes (alleles) segregate together at a frequency that is higher than the predicted frequency by the product of their individual frequencies. This means that the two genes are not independent since they segregate together more frequently than envisaged statistically, and there is thus an independence deficit between alleles located close to each other on the same chromosome.

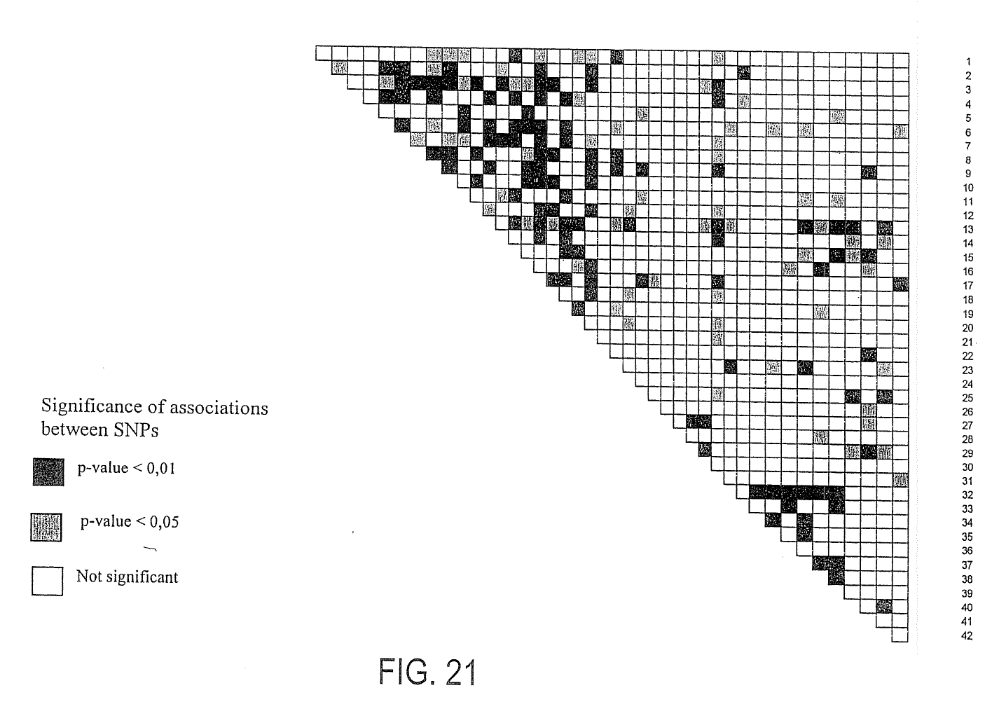

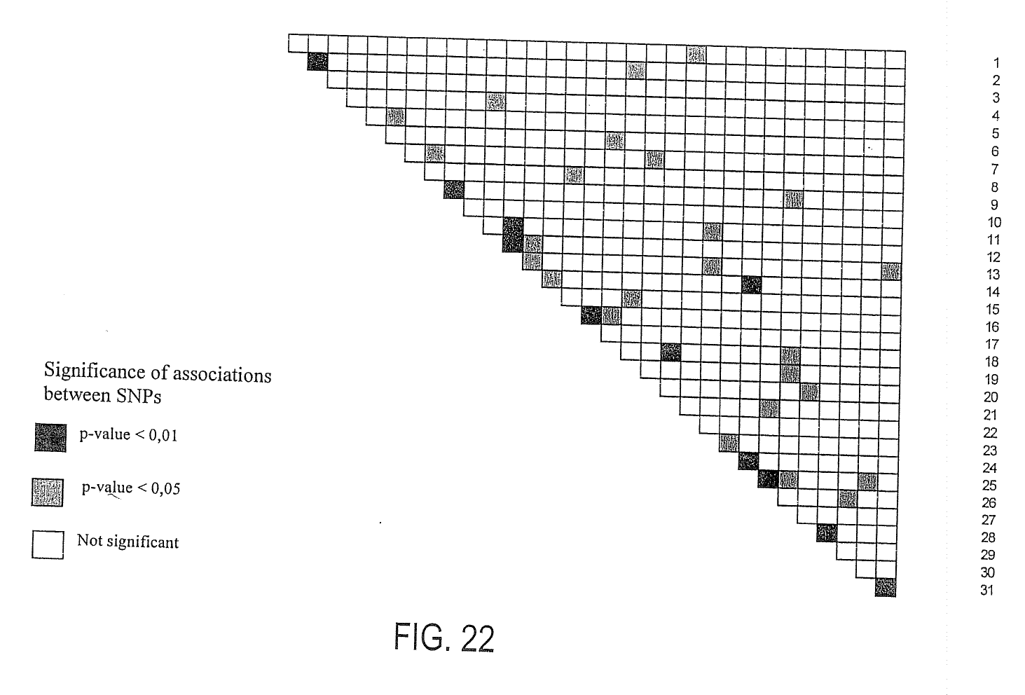

This linkage disequilibrium allows blocks of DNA to be defined which are marked by several markers in which co-segregation of alleles deviates from a co-segregation governed by a single random event. This situation is produced by an absence or deficit of recombination in this block. The size of regions with linkage disequilibrium varies with the chromosomal regions; it appears to extend over 10 kb to 200 kb. The results are shown in FIGS. 21 and 22.

### 8—Comparison of Allele/Genotype Frequencies for Each SNP

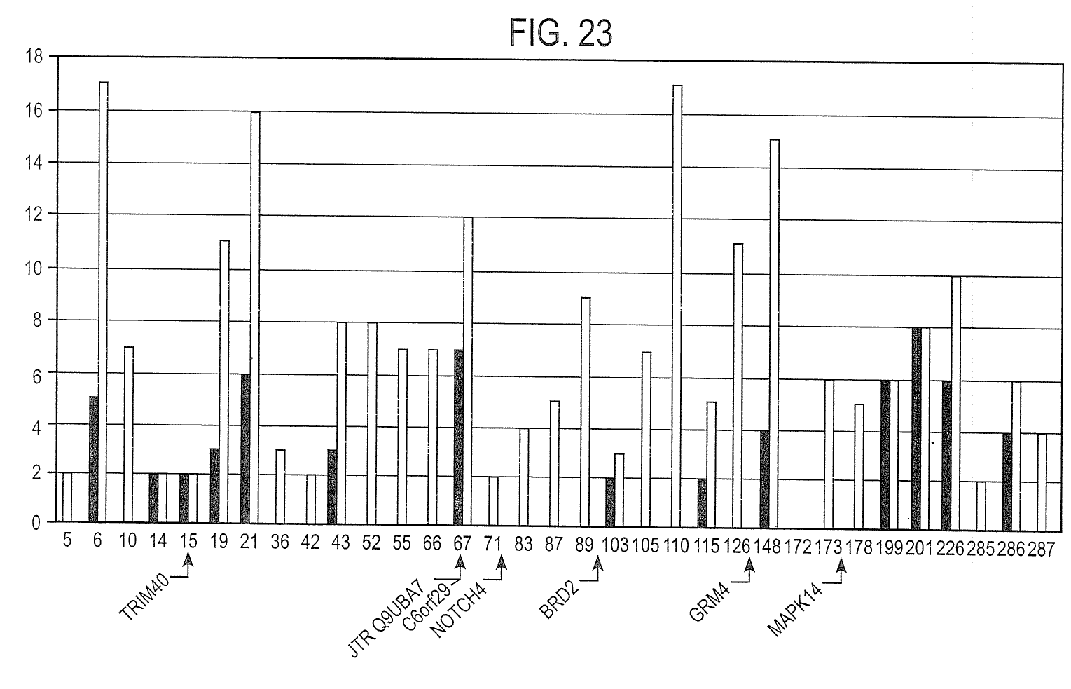

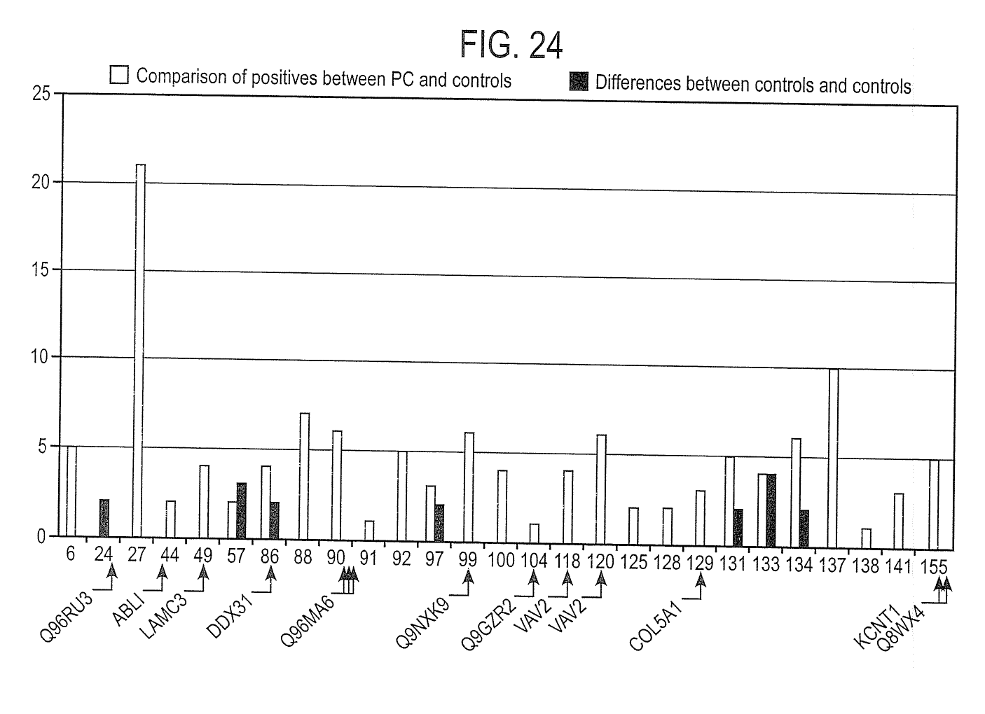

This comparison of allele/genotype frequencies was carried out for each SNP in the premature canities groups (1 to 5 and p) and in the control groups. The results obtained are shown in the following tables and in FIGS. 23 (region A) and 24 (region B).

The “con-con” column shows a comparison between the different groups of control individuals. The “aff” column indicates comparisons for each group of affected persons, against all affected or control groups.

| Chromosome 6, region A | | | | Chromosome 9, region B | | | |
|---|---|---|---|---|---|---|---|
| SNP | Con-con | aff | counts | SNP | Con-con | aff | counts |
| 5 | 0 | 2 | 2 | 6 | 0 | 5 | 5 |
| 6 | 5 | 17 | 22 | 24 | 2 | 0 | 2 |
| 10 | 0 | 7 | 7 | 27 | 0 | 21 | 21 |
| 14 | 2 | 2 | 4 | 44 | 0 | 2 | 2 |
| 15 | 2 | 2 | 4 | 49 | 0 | 4 | 4 |
| 19 | 3 | 11 | 14 | 57 | 3 | 2 | 5 |
| 21 | 6 | 16 | 22 | 86 | 2 | 4 | 6 |
| 36 | 0 | 3 | 3 | 88 | 0 | 7 | 7 |
| 42 | 1 | 2 | 3 | 90 | 0 | 6 | 6 |
| 43 | 3 | 8 | 11 | 91 | 0 | 1 | 1 |
| 52 | 1 | 8 | 9 | 92 | 0 | 5 | 5 |
| 55 | 0 | 7 | 7 | 97 | 2 | 3 | 5 |
| 66 | 1 | 7 | 8 | 99 | 0 | 6 | 6 |
| 67 | 7 | 12 | 19 | 100 | 0 | 4 | 4 |
| 71 | 1 | 2 | 3 | 104 | 0 | 1 | 1 |
| 83 | 0 | 4 | 4 | 118 | 0 | 4 | 4 |
| 87 | 0 | 5 | 5 | 120 | 0 | 6 | 6 |
| 89 | 0 | 9 | 9 | 125 | 0 | 2 | 2 |
| 103 | 2 | 3 | 5 | 128 | 0 | 2 | 2 |
| 105 | 0 | 7 | 7 | 129 | 0 | 3 | 3 |
| 110 | 1 | 17 | 18 | 131 | 2 | 5 | 7 |
| 115 | 2 | 5 | 7 | 133 | 4 | 4 | 8 |
| 126 | 0 | 11 | 11 | 134 | 2 | 6 | 8 |
| 148 | 4 | 15 | 19 | 137 | 0 | 10 | 10 |
| 172 | 0 | 1 | 1 | 138 | 0 | 1 | 1 |
| 173 | 0 | 6 | 6 | 141 | 0 | 3 | 3 |
| 178 | 0 | 5 | 5 | 155 | 0 | 5 | 5 |
| 199 | 6 | 6 | 12 | | | | |
| 201 | 8 | 8 | 16 | | | | |
| 226 | 6 | 10 | 16 | | | | |
| 285 | 0 | 2 | 2 | | | | |
| 286 | 4 | 6 | 10 | | | | |
| 287 | 0 | 4 | 4 | | | | |

### 9—Conclusions

The principle conclusions which can be drawn from these results are as follows:

Firstly, there is a great similarity between the observations made for pool analysis and for individual genotype analysis.

The large “clusters” have been confirmed.

Chromosome 9 reveals an interval in linkage disequilibrium (major cluster) which is strongly associated with the premature canities trait (SNP 418620 to SNP 2526008, position 126,544,533 nt to position 126,745,296 nt, giving a size of 201 kb). This cluster includes the genes DDX31, GTF3C4 and Q96MA6.

The genes or predicted genes identified in the intervals associated with a positive haplotype or a cluster of positive SNPs are as follows:

### Region A

### Haplotype 5-6

HLAG: Start (position on chrom): 39730109 End (position on chrom): 39733287
NT_{—}007592.445: Start (position on chrom): 39750711 End (position on chrom): 39778231
NT_{—}007592.446: Start (position on chrom): 39787841 End (position on chrom): 39809968

### Haplotypes 42-43

NT_{—}007592.506: Start (position on chrom): 40890755 End (position on chrom): 409-45799
NT_{—}007592.507): Start (position on chrom): 40954831 End (position on chrom): 40968983
NT_{—}007592.508: Start (position on chrom): 40977433 End (position on chrom): 41013059

### Haplotypes 66-67

HSPA1B: Start (position on chrom): 41726349 End (position on chrom.): 41728808
G8: Start (position on chrom): 41733475 End (position on chrom): 41738312
NEU1 neuraminidase precursor: Start (position on chrom): 41757894 End (position on chrom): 41761597
NG22: Start (position on chrom): 41761889 End (position on chrom): 41777684
BAT8 ankyrin repeat-containing protein; Mouse Ortholog: Bat8: Start (position on chrom): 41778446 End (position on chrom)-41791599

### Haplotypes 95-96

HLA-DMB: Start (position on chrom): 27217082 End (position on chrom): 27223420
HLA-DMA: Start (position on chrom): 27231061 End (position on chrom): 27235519
BRD2: Start (position on chrom): 27251103 End (position on chrom): 27263747

### Haplotypes 89 (87)

HLA-DQA1: Start (position on chrom): 42482839 End (position on chrom): 42489050
HLA-DQA2: Start (position on chrom.): 42582711 End (position on chrom): 42587664
NT_{—}007592.588: Start (position on chrom): 42498795 End (position on chrom): 42505711

### Haplotypes 126

GRM4 glutamate receptor, metabotropic 4: Start (position on chrom): 43862780 End (position on chrom): 43974595

### Haplotypes 21(18-21)

RNF23: Start (position on chrom): 40229287 End (position on chrom): 402-43110
hypothetical protein FLJ22638: Start (position on chrom): 402-45566 End (position on chrom): 40247261
or (NT_{—}007592.459): Start (position on chrom): 40245578 End (position on chrom): 40369534
NT_{—}007592.457): Start (position on chrom): 40160064 End (position on chrom): 40218336

### Region B

### Haplotype 27

FREQ: PubMed on Product: frequenin homolog/Mouse Ortholog: Freq
Start (position on chrom): 124490317 End (position on chrom): 124554366
NT_{—}030046.18 Start (position on chrom): 124458070 End (position on chrom): 124489558
NT_{—}030046.17: Start (position on chrom): 124371672 End (position on chrom): 124452860

### Haplotype 97-100

GTF3C5. PubMed on Product: general transcription factor IIIC, polypeptide 5
Start (position on chrom): 127480920 End (position on chrom): 127508694
CEL: PubMed on Product: carboxyl ester lipase (bile salt-stimulated) Start (position on chrom): 127512178 End (position on chrom): 127522054
CELL: PubMed on Product: carboxyl ester lipase-like (bile salt-stimulated) Start (position on chrom): 127532733 End (position on chrom): 127537549
FS: PubMed on Product: Forssman synthetase Start (position on chrom): 127603661 End (position on chrom): 127614093
ABO blood group (transferase A, alpha): Start (position on chrom): 127907180 End (position on chrom): 127924298

### Haplotypes 86-92

### BARHL1

### DDX31

### GTF3C4

Q96MA6 (Adenylate cyclase)
New Analysis of Region Corresponding to Haplotype 86-92, with New SNPs, Single Nucleotide Polymorphisms

This region on chromosome 9 was subjected to a new analysis with a collection of new SNPs which could cover the region more densely (1 SNP every 2 to 3 kb over this region of 120 kb).

Individual genotyping carried out with these SNPs showed that there were 2 highly positive genes out of the 4: DDX31 and GTF3C4.

### Example 3

### Analysis of Regions of Interest on Chromosomes 3, 5 and 11 with SNPs, Single Nucleotide Polymorphisms

The same pool allelotyping experiments were carried out on the regions of interest on chromosomes 3, 5 and 11 as defined in Example 1, selecting the SNPs in the gene regions or near thereto.

This analysis demonstrated the following genes on this region:

The coordinates are given as a function of Build 30 (June 2002).

Chromosome 3 (region C):

### C1 (41527287-41677819).

hypothetical protein KIAA1042

CCK: gastrin/cholecystokinin type b receptor (cck-b receptor)

### C2: (52846896-52913941):

CACNAID: voltage-dependent 1-type calcium channel alpha-Id subunit

### C3: (55638663-56042041):

ARHGEF3 rho guanine nucleotide exchange factor 3; rhogef protein; 59.8 kda protein; exchange factor found in platelets and leukemic and neuronal tissues, xpln.

Hypothetical protein AL133097

Chromosome 5 (region D):

### D1 (136479801-137007868):

KLHL3: kelch-like protein 3

HNRP A0: heterogeneous nuclear ribonucleoprotein a0 (hnmp a0).

### D2(137542040-137771805)

CDC25C: map/microtubule affinity-regulating kinase 3 (ec 2.7.1.27)

EGR1: early growth response protein 1 (egr-1) (krox-24 protein)

C5orf6: predicted

C5orf7: predicted

LOC51308: predicted

ETF1: eukaryotic peptide chain release factor subunit 1 (erf1)

HSP A9B: stress-70 protein, mitochondrial precursor

### D3(139931847-140118601)

PCDHA1 to PCDHA13: protocadherin. alpha 1 precursor to protocadherin alpha 1 precursor

### D4(149518721-149586774)

CSFIR: Macrophage Colony Stimulating Factor I Receptor Precursor

RPL7: 60s ribosomal protein 17

PDGFRB: beta platelet-derived growth factor receptor precursor (ec 2.7.1.112)

### D5(149793126-149995886)

TCOFC1: Treacle Protein (Treacher Collins Syndrome Protein).

AL133039: predicted

CD74: hla class ii histocompatibility antigen, gamma chain

RPS14: 40s ribosomal protein s14

NDST1: Heparan Sulfate N-Deacetylase/N-Sulfotransferase (Ec 2.8.2.8)

### D6(151235618-151373121)

G3BP: ras-gtpase-activating protein binding protein 2

GLRAI: glycine receptor alpha-I chain precursor

### D7(153463449-153854650)

C5 orf3: predicted

MFAP3: microfibril-associated glycoprotein 3 precursor

GALNT10: putative udp-galnac:polypeptide n-acetylgalactosaminyltransferase

FLJ11715: predicted

Chromosome 11 (region E):

### E1 (108893187-108944206)

GUCY1A2: guanylate cyclase soluble, alpha-2 chain (ec 4.6.1.2)

### E2(110056711-110546142)

CUL5: vasopressin-activated calcium-mobilizing receptor (vacm-1) (cullin homolog 5)

ACAT1: acetyl-coa acetyltransferase, mitochondrial precursor (ec 2-3.1.9)

NPAT: nuclear protein, ataxia-telangiectasia locus; e 14 gene;

ATM: serine-protein kinase atm (ec 2.7.1.37) (ataxia telangiectasia mutated

AF035326: predicted

AF035327: predicted

AF035328: predicted

BC029536: predicted

### E3(115527211-115745012)

FLJ20535

DRD2: d(2) dopamine receptor

ENS303941: predicted

### E4 (117397672-117752160):

IGSF4: immunoglobulin superfamily, member 4; nectin-like protein 2

### E5 (118532530-118685957)

No known gene

### E6 (119417270-119469358)

LOC51092: predicted

BC010946: predicted

TAGLN: transgelin (smooth muscle protein 22-alpha) (sm22-alpha) (ws3-10) (22 kda actin-binding protein).

PCSK7: proprotein conveltase subtilisin/kexin type 7 precursor (ec 3.4.21.-)

ENS300650: predicted

### Example 4

### Examples of Compositions

Hair Lotion

| | |
|---|---|
| DNA fragment from one of the genes of the invention | 0.5 g |
| belonging to the chromosomal zone included between | |
| markers D6S1629 and D6S257 propylene glycol | 20 g |
| 95° ethanol | 30 g |
| water | qsp 100 g |

This lotion was applied daily to the zones to be treated, preferably to the whole scalp, for at least 10 days and preferably 1 to 2 months.

A reduction in the appearance of white or gray hairs and re-pigmentation of gray hair was observed.

Treatment Shampoo

| | |
|---|---|
| DNA fragment from one of the genes of the invention | 1.5 g |
| belonging to the chromosomal zone included between | |
| the D9S290 marker and the long arm telomere | |
| polyglyceryl 3-hydroxylarylether | 26 g |
| hydroxypropyl cellulose sold as Klucell G by Hercules | 2 g |
| preservatives | qps |
| 95° ethanol | 50 g |
| water | qsp 100 g |

This shampoo was used at each wash, leaving it on the hair for about one minute. Long term use, of the order of two months, resulted in progressive re-pigmentation of gray hair. This shampoo could also be used preventatively to retard whitening of the hair.

Treatment Gel

| | |
|---|---|
| DNA fragment from one of the genes of the invention | 0.75 g |
| belonging to the chromosomal zone included between | |
| markers D6S1629 and D6S257 | |
| essential eucalyptus oils | 1 g |
| econozole | 0.2 g |
| lauryl polyglyceryl 6-cetearyl glycoether | 1.9 g |
| preservatives | qs |
| carbopol 934P, sold by B F Goodrich Corporation | 0.3 g |
| neutralizing agent | qs pH 7 |
| water | qsp 100 g |

This gel was applied to the zones to be treated twice daily (morning and evening) with a finishing massage. After three months application, repigmentation of hair was observed in the treated zone.

### REFERENCES

E Lander and L Kruglyak: Genetic dissociation of complex traits: guidelines for interpreting and reporting linkage results. Nat. Genet. 11 (3): 241-247, 1995.

1. A method for screening molecules that can modulate the function of a polynucleotide fragment, said fragment comprising at least 18 consecutive nucleotides the sequence of which corresponds to all or part of a gene on human chromosome 9, said gene being chosen amongst the DDX31 gene and the GTF3C4 gene, comprising the steps of:
bringing the molecule to be tested into the presence of the polynucleotide fragment; and
detecting a variation in a parameter linked to the function of said fragment, for the identification of an agent for cosmetic or therapeutic purposes, in the field of pigmentation.

2. The method of claim 1, wherein the detection step is the detection of any binding of said molecule to the polynucleotide fragment.

3. The method of claim 2, wherein said detection of any binding is demonstrated by a ligand binding detection method.

4. The method of claim 1, for the detection of an inhibitor for the function of the DDX31 gene or the GTF3C4 gene.

5. The method of claim 1, for the detection of an agent promoting or inhibiting the transcription of the DDX31 gene or the GTF3C4 gene.

6. A method for screening molecules that can modulate the function of the expression product of a polynucleotide fragment, said fragment comprising at least 18 consecutive nucleotides the sequence of which corresponds to all or part of a gene on human chromosome 9, said gene being chosen amongst the DDX31 gene and the GTF3C4 gene, comprising the steps of:
bringing the molecule to be tested into the presence of the expression product; and
detecting a variation in a parameter linked to the function of said expression product, for cosmetic or therapeutic purposes, in the field of pigmentation.

7. The method of claim 6, wherein the detection step is the detection of any binding of said molecule to the expression product.

8. The method of claim 7, wherein said detection of any binding is demonstrated by a ligand binding detection method.

9. The method of claim 6, for the detection of an inhibitor for the functions of an expression product of the DDX31 gene or the GTF3C4 gene.

10. The method of claim 6, wherein said expression product is an RNA molecule derived from transcription of the DDX31 gene or of the GTF3C4 gene.

11. The method of claim 6, wherein said expression product is a polypeptide derived from translation of the DDX31 gene or of the GTF3C4 gene.

12. The method of claim 6, wherein said molecule is an antisense RNA molecule.

13. The method of claim 6, wherein said molecule is an antibody.

14. Use of at least one marker selected from SNP 418620, rs302919, 913705, 932886, 429269 and 2526008, for the determination of genes involved in the pigmentation of the skin or phanera.
